(19) United States
(12) Patent Application Publication
(10) Pub. No.: US 2021/0238184 A1
(43) Pub. Date: Aug. 5, 2021

(54) THERAPEUTIC COMPOUNDS

(71) Applicant: Genentech, Inc., South San Francisco, CA (US)

(72) Inventors: Jason Robert Zbieg, Montara, CA (US); Paul Powell Beroza, Belmont, CA (US); James John Crawford, South San Francisco, CA (US)

(73) Assignee: Genentech, Inc., South San Francisco, CA (US)

(21) Appl. No.: 17/105,088

(22) Filed: Nov. 25, 2020

Related U.S. Application Data
(63) Continuation of application No. PCT/US2019/034660, filed on May 30, 2019.
(60) Provisional application No. 62/678,567, filed on May 31, 2018.

(51) Int. Cl. C07D 487/04

(52) U.S. Cl. CPC C07D 487/04

(57) ABSTRACT

The present disclosure relates to compounds of formula (I):

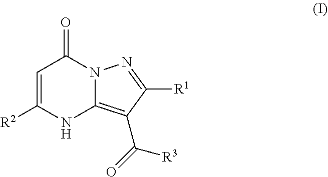

and pharmaceutically acceptable salts thereof, wherein R¹-R³ have any of the values defined herein, and compositions and uses thereof. The compounds are useful as inhibitors of the YAP:TEAD protein:protein interaction. Also included are pharmaceutical compositions comprising a compound of formula (I) or a pharmaceutically acceptable salt thereof, and methods of using such compounds and salts in the treatment of various YAP:TEAD-mediated disorders, including cancer.

### CROSS REFERENCE TO RELATED APPLICATIONS

This application is a continuation of International Application No. PCT/US2019/034660 having an International filing date of May 30, 2019, which claims priority to U.S. Provisional Application No. 62/678,567, filed May 31, 2018, all of which are incorporated herein by reference in their entirety.

### BACKGROUND

The Hippo pathway is a signaling pathway that regulates cell proliferation and cell death and determines organ size. The pathway is believed to play a role as a tumor suppressor in mammals, and disorders of the pathway are often detected in human cancers. The pathway is involved in and/or may regulate the self-renewal and differentiation of stem cells and progenitor cells. In addition, the Hippo pathway may be involved in wound healing and tissue regeneration. Furthermore, it is believed that as the Hippo pathway cross-talks with other signaling pathways such as Wnt, Notch, Hedgehog, and MAPK/ERK, it may influence a wide variety of biological events, and that its dysfunction could be involved in many human diseases in addition to cancer.

The Hippo signaling pathway core consists of a cascade of kinases (Hippo-MST1-2 being upstream of Lats 1-2 and NDRI-2) leading to the phosphorylation of two transcriptional co-activators, YAP (Yes-Associated Protein) and TAZ (Transcription co-activator with PDZ binding motif or tafazzin). Non-phosphorylated, activated YAP is translocated into the cell nucleus where its major target transcription factors are the four proteins of the TEAD-domain-containing family (TEAD1-TEAD4, collectively “TEAD”). YAP together with TEAD (or other transcription factors such as Smad1, RUNX, ErbB4 and p73) has been shown to induce the expression of a variety of genes, including connective tissue growth factor (CTGF), Gli2, Birc5, Birc2, fibroblast growth factor 1 (FGF1), and amphiregulin (AREG). Like YAP, non-phosphorylated TAZ is translocated into the cell nucleus where it interacts with multiple DNA-binding transcription factors, such as peroxisome proliferator-activated receptor γ (PPARγ), thyroid transcription factor-1 (TTF-1), Pax3, TBX5, RUNX, TEAD1 and Smad2/3/4. Many of the genes activated by YAP/TAZ-transcription factor complexes mediate cell survival and proliferation. Therefore, under some conditions YAP and/or TAZ acts as an oncogene and the Hippo pathway acts as a tumor suppressor.

Because the Hippo signaling pathway is a regulator of animal development, organ size control and stem cell regulation, it has been implicated in cancer development. In vitro, the overexpression of YAP or TAZ in mammary epithelial cells induces cell transformation, through interaction of both proteins with the TEAD family of transcription factors. Increased YAP/TAZ transcriptional activity induces oncogenic properties such as epithelial-mesenchymal transition and was also shown to confer stem cells properties to breast cancer cells. In vivo, in mouse liver, the overexpression of YAP or the genetic knockout of its upstream regulators MST1-2 triggers the development of hepatocellular carcinomas. Furthermore, when the tumor suppressor NF2 is inactivated in the mouse liver, the development of hepatocellular carcinomas can be blocked completely by the co-inactivation of YAP.

It is believed that deregulation of the Hippo tumor suppressor pathway is a major event in the development of a wide range of cancer types and malignancies.

Hence, pharmacological targeting of the Hippo cascade through inhibition of YAP, TAZ, TEAD, and/or the YAP:TEAD protein-protein interaction would be a valuable approach for the treatment of cancers that harbor functional alterations of this pathway.

### SUMMARY

In some embodiments, a compound of formula (I), stereoisomers thereof, tautomers thereof, and salts thereof are provided:

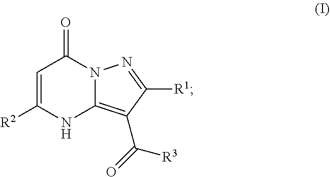

wherein:

R¹ is selected from the group consisting of hydrogen, halogen, C₁₋₁₀alkyl, and C₁₋₁₀haloalkyl;

R² is C₅₋₁₀aryl or C₅₋₁₀heteroaryl;

R³ is OR⁹ or NR¹⁰R¹¹;

wherein:

when R² is C₅₋₁₀heteroaryl and R³ is NR¹⁰R¹¹, then each of R¹⁰ and R¹¹ is not hydrogen; and

when R³ is OR⁹, then R² is not pyridyl;

R⁹ is selected from the group consisting of unsubstituted or substituted C₁₋₁₀alkyl, unsubstituted or substituted C₃₋₈cycloalkyl, unsubstituted or substituted C₆₋₁₀aryl, and unsubstituted or substituted C₅₋₁₀heteroaryl; wherein each R⁹ may be optionally substituted with one to five R^{e} groups;

R¹⁰ and R¹¹ are each independently selected from the group consisting of hydrogen, unsubstituted or substituted C₁₋₁₀alkyl, unsubstituted or substituted C₃₋₆cycloalkyl, unsubstituted or substituted C₆₋₁₀aryl, unsubstituted or substituted C₅₋₁₀heteroaryl, unsubstituted or substituted CR^{f}₂—C₆₋₁₀aryl, and R¹⁰ and R¹¹ cyclized to form an unsubstituted or substituted ring having 3-8 ring members; wherein each R¹⁰, R¹¹, and the ring having 3-8 ring members may be optionally substituted with one to five R^{e} groups;

R^{e} is selected from the group consisting of halogen, OH, C₁₋₁₀alkyl, O—C₁₋₁₀alkyl, C₁₋₁₀haloalkyl, O—C₁₋₁₀ haloalkyl, cyano, C₃₋₈cycloalkyl, C₆₋₁₀aryl, and NR^{g}R^{h};

R^{f} is selected from the group consisting of hydrogen, unsubstituted or substituted C₁₋₁₀alkyl, unsubstituted or substituted C₁₋₁₀haloalkyl, unsubstituted or substituted C₃₋₈cycloalkyl; wherein each R^{f} may be optionally substituted with one to five R^{e} groups; and

R^{g} and R^{h} are each independently selected from the group consisting of C₁₋₁₀alkyl, C₃₋₈cycloalkyl, and C₆₋₁₀aryl,

or a pharmaceutically acceptable salt thereof.

In embodiments, R² is

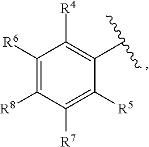

wherein

R⁴, R⁵, R⁶, and R⁷ are each independently selected from the group consisting of hydrogen, halogen, C₁₋₁₀alkyl, C₁₋₁₀haloalkyl, O—C₁₋₁₀alkyl, and NR^{a}R^{b}, wherein R^{a} and R^{b} are each independently selected from the group consisting of C₁₋₁₀alkyl, C₃₋₈cycloalkyl, and C₆₋₁₀aryl;

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted O—C₁₋₁₀haloalkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, unsubstituted or substituted C₅₋₁₀heteroaryl; and unsubstituted or substituted NR^{c}R^{d}, wherein R^{c} and R^{d} are each independently selected from the group consisting of unsubstituted or substituted C₁₋₁₀alkyl, unsubstituted or substituted C₃₋₈cycloalkyl, and C₆₋₁₀aryl, wherein each R^{c} and R^{d} may be optionally substituted with one to five R^{e} groups; and wherein each R⁸ may be optionally substituted with one to five R^{e} groups selected from the group consisting of halogen, OH, C₁₋₁₀alkyl, O—C₁₋₁₀alkyl, C₁₋₁₀haloalkyl, O—C₁₋₁₀haloalkyl, cyano, C₃₋₈cycloalkyl, C₆₋₁₀aryl, and NR^{g}R^{h}, wherein R^{g} and R^{h} are each independently selected from the group consisting of C₁₋₁₀alkyl, C₃₋₈cycloalkyl, and C₆₋₁₀aryl.

In some embodiments, a compound of formula (I), stereoisomers thereof, tautomers thereof, and salts thereof are provided:

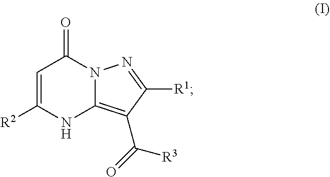

wherein:

R¹ is selected from the group consisting of hydrogen, halogen, C₁₋₁₀alkyl, and C₁₋₁₀haloalkyl;

R² is

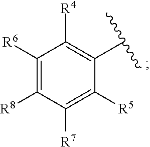

R³ is OR⁹ or NR¹⁰R¹¹;

R⁴, R⁵, R⁶, and R⁷ are each independently selected from the group consisting of hydrogen, halogen, C₁₋₁₀alkyl, C₁₋₁₀haloalkyl, O—C₁₋₁₀alkyl, and NR^{a}R^{b};

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted O—C₁₋₁₀haloalkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, unsubstituted or substituted C₅₋₁₀heteroaryl; and unsubstituted or substituted NR^{c}R^{d}; wherein each R⁸ may be optionally substituted with one to five R^{e} groups;

R⁹ is selected from the group consisting of unsubstituted or substituted C₁₋₁₀alkyl, unsubstituted or substituted C₃₋₈cycloalkyl, unsubstituted or substituted C₆₋₁₀aryl, and unsubstituted or substituted C₅₋₁₀heteroaryl; wherein each R⁹ may be optionally substituted with one to five R^{e} groups; r¹⁰ and R¹¹ are each independently selected from the group consisting of hydrogen, unsubstituted or substituted C₁₋₁₀alkyl, unsubstituted or substituted C₃₋₆cycloalkyl, unsubstituted or substituted C₆₋₁₀aryl, unsubstituted or substituted C₅₋₁₀heteroaryl, unsubstituted or substituted CR^{f}₂—C₆₋₁₀aryl, and R¹⁰ and R¹¹ cyclized to form an unsubstituted or substituted ring having 3-8 ring members; wherein each R¹⁰, R¹¹, and the ring having 3-8 ring members may be optionally substituted with one to five R^{e} groups;

R^{a} and R^{b} are each independently selected from the group consisting of C₁₋₁₀alkyl, C₃₋₈cycloalkyl, and C₆₋₁₀aryl;

R^{c} and R^{d} are each independently selected from the group consisting of unsubstituted or substituted C₁₋₁₀alkyl, unsubstituted or substituted C₃₋₈cycloalkyl, and C₆₋₁₀aryl; wherein each R^{c} and R^{d} may be optionally substituted with one to five R^{e} groups;

R^{e} is selected from the group consisting of halogen, OH, C₁₋₁₀alkyl, O—C₁₋₁₀alkyl, C₁₋₁₀haloalkyl, O—C₁₋₁₀haloalkyl, cyano, C₃₋₈cycloalkyl, C₆₋₁₀aryl, and NR^{g}R^{h};

R^{f} is selected from the group consisting of hydrogen, unsubstituted or substituted C₁₋₁₀alkyl, unsubstituted or substituted C₁₋₁₀haloalkyl, unsubstituted or substituted C₃₋₈cycloalkyl; wherein each R^{f} may be optionally substituted with one to five R^{e} groups; and

R^{g} and R^{h} are each independently selected from the group consisting of C₁₋₁₀alkyl, C₃₋₈cycloalkyl, and C₆₋₁₀aryl,

or a pharmaceutically acceptable salt thereof.

Some other embodiments provide pharmaceutical compositions comprising a compound described above, or a pharmaceutically acceptable salt thereof, and a pharmaceutically acceptable carrier, diluent or excipient.

Some other embodiments provide pharmaceutical compositions comprising a compound described above, and a therapeutically inert carrier.

Some other embodiments provide a compound as described above, or a pharmaceutically acceptable salt thereof, for use in medical therapy.

Some other embodiments provide a compound as described above, or a pharmaceutically acceptable salt thereof, for use as a therapeutically active substance.

Some other embodiments provide a compound as described above, or a pharmaceutically acceptable salt thereof, for the treatment or prophylaxis of cancer.

Some other embodiments provide a compound as described above, or a pharmaceutically acceptable salt thereof, for the preparation of a medicament for the treatment or prophylaxis of cancer.

Some other embodiments provide a method for treating cancer in a mammal comprising, administering a therapeutically effective amount of a compound as described above, or a pharmaceutically acceptable salt thereof, to the mammal.

Some other embodiments provide a compound as described above, or a pharmaceutically acceptable salt thereof, for modulating the YAP:TEAD protein-protein interaction.

Some other embodiments provide a compound as described above, or a pharmaceutically acceptable salt thereof, for the treatment or prophylaxis of a disease or condition mediated by YAP:TEAD activity.

Some other embodiments provide a use of a compound as described above, or a pharmaceutically acceptable salt thereof, for the preparation of a medicament for the treatment or prophylaxis of a disease or condition that is mediated by YAP:TEAD activity.

Some other embodiments provide a method for modulating YAP:TEAD activity, comprising contacting YAP:TEAD with a compound as described above, or a pharmaceutically acceptable salt thereof.

Some other embodiments provide a method for treating a disease or condition mediated by YAP:TEAD activity in a mammal, comprising administering a therapeutically effective amount of a compound as described above, or a pharmaceutically acceptable salt thereof, to the mammal.

### DETAILED DESCRIPTION

### I. Definitions

Unless otherwise indicated, the following specific terms and phrases used in the description and claims are defined as follows:

The terms “moiety” and “substituent” refer to an atom or group of chemically bonded atoms that is attached to another atom or molecule by one or more chemical bonds thereby forming part of a molecule.

The term “substituted” refers to the replacement of at least one of hydrogen atom of a compound or moiety with another substituent or moiety. Examples of such substituents include, without limitation, halogen, —OH, —CN, oxo, alkoxy, alkyl, aryl, heteroaryl, haloalkyl, haloalkoxy, cycloalkyl and heterocycle. For example, the term “alkyl substituted by halogen” refers to the fact that one or more hydrogen atoms of a alkyl (as defined below) is replaced by one or more halogen atoms (e.g., trifluoromethyl, difluoromethyl, fluoromethyl, chloromethyl, etc.).

The term “alkyl” refers to an aliphatic straight-chain or branched-chain saturated hydrocarbon moiety having 1 to 20 carbon atoms unless provided otherwise. For example, in particular embodiments, the alkyl has 1 to 10 carbon atoms. In particular embodiments the alkyl has 1 to 6 carbon atoms. Alkyl groups may be optionally substituted independently with one or more substituents described herein.

The term “alkoxy” denotes a group of the formula —O—R′, wherein R′ is an alkyl group. Alkoxy groups may be optionally substituted independently with one or more substituents described herein. Examples of alkoxy moieties include methoxy, ethoxy, isopropoxy, and tert-butoxy.

“Aryl” means a cyclic aromatic hydrocarbon moiety having a mono-, bi- or tricyclic aromatic ring of 5 to 16 carbon ring atoms unless provided otherwise. For example, in particular embodiments the aryl has 6 to 10 carbon atoms. Bicyclic aryl ring systems include fused bicyclics having two fused five-membered aryl rings (denoted as 5-5), having a five-membered aryl ring and a fused six-membered aryl ring (denoted as 5-6 and as 6-5), and having two fused six-membered aryl rings (denoted as 6-6). The aryl group can be optionally substituted as defined herein. Examples of aryl moieties include, but are not limited to, phenyl, naphthyl, phenanthryl, fluorenyl, indenyl, pentalenyl, azulenyl, and the like. The term “aryl” also includes partially hydrogenated derivatives of the cyclic aromatic hydrocarbon moiety provided that at least one ring of the cyclic aromatic hydrocarbon moiety is aromatic, each being optionally substituted.

The term “heteroaryl” denotes an aromatic heterocyclic mono-, bi- or tricyclic ring system of 5 to 16 ring atoms unless provided otherwise, comprising 1, 2, 3 or 4 heteroatoms selected from N, O and S, the remaining ring atoms being carbon. For example, in some aspects, monocyclic heteroaryl rings may be 5-6 membered. In some aspects, heteroaryl rings may contain 5 to 10 carbon atoms. Bicyclic heteroaryl ring systems include fused bicyclics having two fused five-membered heteroaryl rings (denoted as 5-5), having a five-membered heteroaryl ring and a fused six-membered heteroaryl ring (denoted as 5-6 and 6-5), and having two fused six-membered heteroaryl rings (denoted as 6-6). The heteroaryl group can be optionally substituted as defined herein. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, or quinoxalinyl.

The terms “halo”, “halogen” and “halide”, which may be used interchangeably, refer to a substituent fluoro, chloro, bromo, or iodo.

The term “haloalkyl” denotes an alkyl group wherein one or more of the hydrogen atoms of the alkyl group has been replaced by the same or different halogen atoms, particularly fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl or trifluoromethyl.

“Cycloalkyl” means a saturated or partially unsaturated carbocyclic moiety having mono-, bi- (including bridged bicyclic) or tricyclic rings and 3 to 10 carbon atoms in the ring unless provided otherwise. For example, in particular embodiments cycloalkyl contains from 3 to 8 carbon atoms (i.e., (C₃-C₈)cycloalkyl). In other particular embodiments cycloalkyl contains from 3 to 6 carbon atoms (i.e., (C₃-C₆)cycloalkyl). Examples of cycloalkyl moieties include, but are not limited to, cyclopropyl, cyclobutyl, cyclopentyl, cyclohexyl, cycloheptyl, and partially unsaturated (cycloalkenyl) derivatives thereof (e.g. cyclopentenyl, cyclohexenyl, and cycloheptenyl), bicyclo[3.1.0]hexanyl, bicyclo[3.1.0]hexenyl, bicyclo[3.1.1]heptanyl, and bicyclo[3.1.1]heptenyl. The cycloalkyl moiety can be attached in a “spirocycloakyl” fashion such as “spirocyclopropyl”:

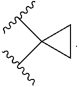 [SMILES: *C1(*)CC1]

The cycloalkyl moiety can optionally be substituted with one or more substituents.

“Heterocycle” or “heterocyclyl” refers to a 3, 4, 5, 6 and 7-membered monocyclic, 7, 8, 9 and 10-membered bicyclic (including bridged bicyclic) or 10, 11, 12, 13, 14 and 15-membered bicyclic heterocyclic moiety, unless provided otherwise, that is saturated or partially unsaturated, and has one or more (e.g., 1, 2, 3 or 4 heteroatoms selected from oxygen, nitrogen and sulfur in the ring with the remaining ring atoms being carbon. For example, in particular embodiments heterocycle or heterocyclyl refers to a 4, 5, 6 or 7-membered heterocycle. In some aspects, the heterocycle is a heterocycloalkyl. When used in reference to a ring atom of a heterocycle, a nitrogen or sulfur may also be in an oxidized form, and a nitrogen may be substituted with one or more groups such as C₁-C₆alkyl. The heterocycle can be attached to its pendant group at any heteroatom or carbon atom that results in a stable structure. Any of the heterocycle ring atoms can be optionally substituted with one or more substituents described herein. Examples of such saturated or partially unsaturated heterocycles include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The term the term heterocycle also includes groups in which a heterocycle is fused to one or more aryl, heteroaryl, or cycloalkyl rings, such as indolinyl, 3H-indolyl, chromanyl, azabicyclo[2.2.1]heptanyl, azabicyclo[3.1.0]hexanyl, azabicyclo[3.1.1]heptanyl, octahydroindolyl, or tetrahydroquinolinyl.

The term “fused bicyclic” denotes a ring system including two fused rings, including bridged cycloalkyl and bridged heterocycloalkyl as defined elsewhere herein. The rings are each independently, aryl, heteroaryl, cycloalkyl, and heterocycle. In some aspects, the rings are each independently, C₅₋₆aryl, 5-6 membered heteroaryl, C₃₋₆cycloalkyl, and 4-6 membered heterocycle. Non-limiting examples of fused bicyclic ring systems include C₅₋₆aryl-C₅₋₆aryl, C₅₋₆aryl-4-6 membered heteroaryl, and C₅₋₆aryl-C₅₋₆cycloalkyl.

Unless otherwise indicated, the term “hydrogen” or “hydro” refers to the moiety of a hydrogen atom (—H) and not H₂.

In the description herein, if the stereochemistry of a structure or a portion of a structure is not indicated with, for example, bold wedged, or dashed lines, the structure or portion of the structure is to be interpreted as encompassing all stereoisomers of it. In some cases, however, where more than one chiral center exists, the structures and names may be represented as single enantiomers to help describe the relative stereochemistry.

Unless otherwise indicated, the term “a compound of the formula” or “a compound of formula” or “compounds of the formula” or “compounds of formula” refers to any compound selected from the genus of compounds as defined by the formula (including any pharmaceutically acceptable salt or ester of any such compound if not otherwise noted).

The term “pharmaceutically acceptable salts” refers to those salts which retain the biological effectiveness and properties of the free bases or free acids, which are not biologically or otherwise undesirable. As used herein, “pharmaceutically acceptable” refers to a carrier, diluent or excipient that is compatible with the other ingredients of the formulation and not deleterious to the recipient thereof. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcy stein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The compounds of the present invention can be present in the form of pharmaceutically acceptable salts. Another embodiment provides non-pharmaceutically acceptable salts of a compound of formula (I), which can be useful as an intermediate for isolating or purifying a compound of formula (I). The compounds of the present invention can also be present in the form of pharmaceutically acceptable esters (e.g., the methyl and ethyl esters of the acids of formula (I) to be used as prodrugs). The compounds of the present invention can also be solvated, e.g. hydrated. The solvation can be effected in the course of the manufacturing process or can take place e.g. as a consequence of hygroscopic properties of an initially anhydrous compound of formula (I).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture”. In certain embodiments the compound is enriched by at least about 90% by weight with a single diastereomer or enantiomer. In other embodiments the compound is enriched by at least about 95%, 98%, or 99% by weight with a single diastereomer or enantiomer.

Certain compounds of the present invention possess asymmetric carbon atoms (optical centers) or double bonds; the racemates, diastereomers, regioisomers and individual isomers (e.g., separate enantiomers) are all intended to be encompassed within the scope of the present invention.

The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. In some instances, the stereochemistry has not been determined or has been provisionally assigned. Many organic compounds exist in optically active forms, i.e., they have the ability to rotate the plane of plane polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity. Enantiomers may be separated from a racemic mixture by a chiral separation method, such as supercritical fluid chromatography (SFC). Assignment of configuration at chiral centers in separated enantiomers may be tentative, and depicted in compounds (1), (m) and (n) for illustrative purposes, while stereochemistry is definitively established, such as from x-ray crystallographic data.

The term “a therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 0.1 mg to about 5,000 mg, 1 mg to about 1,000 mg, or 1 mg to 100 mg may be appropriate, although the lower and upper limits may be exceeded when indicated. The daily dosage can be administered as a single dose or in divided doses, or for parenteral administration, it may be given as continuous infusion.

The terms “pharmaceutically acceptable carrier”, “pharmaceutically acceptable carrier, adjuvant, or vehicle”, or “therapeutically inert carrier” may be used interchangeably throughout and are intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Useful pharmaceutically acceptable carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in R^{e} mington's Pharmaceutical Sciences by E. W. Martin. Such compositions will, in any event, contain an effective amount of the active compound together with a suitable carrier so as to prepare the proper dosage form for proper administration to the recipient.

The term “patient” or “individual” as used herein, refers to an animal, such as a mammal, such as a human. In one embodiment, patient or individual refers to a human.

In the practice of the method of the present invention, a therapeutically effective amount of any one of the compounds of this invention or a combination of any of the compounds of this invention or a pharmaceutically acceptable salt or ester thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

### II. Generic and Subgeneric Formulae of Disclosed Compounds

Provided herein are compounds of formula (I), stereoisomers thereof, tautomers thereof, and salts thereof:

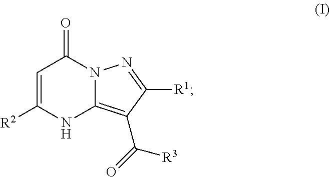

wherein:

R¹ is selected from the group consisting of hydrogen, halogen, C₁₋₁₀alkyl, and C₁₋₁₀haloalkyl;

R² is C₅₋₁₀aryl or C₅₋₁₀heteroaryl;

R³ is OR⁹ or NR¹⁰R¹¹;

wherein:

when R² is C₅₋₁₀heteroaryl and R³ is NR¹⁰R¹¹, then each of R¹⁰ and R¹¹ is not hydrogen; and

when R³ is OR⁹, then R² is not pyridyl;

R⁹ is selected from the group consisting of unsubstituted or substituted C₁₋₁₀alkyl, unsubstituted or substituted C₃₋₈cycloalkyl, unsubstituted or substituted C₆₋₁₀aryl, and unsubstituted or substituted C₅₋₁₀heteroaryl; wherein each R⁹ may be optionally substituted with one to five R^{e} groups;

R¹⁰ and R¹¹ are each independently selected from the group consisting of hydrogen, unsubstituted or substituted C₁₋₁₀alkyl, unsubstituted or substituted C₃₋₆cycloalkyl, unsubstituted or substituted C₆₋₁₀aryl, unsubstituted or substituted C₅₋₁₀heteroaryl, unsubstituted or substituted CR^{f}₂—C₆₋₁₀aryl, and R¹⁰ and R¹¹ cyclized to form an unsubstituted or substituted ring having 3-8 ring members; wherein each R¹⁰, R¹¹, and the ring having 3-8 ring members may be optionally substituted with one to five R^{e} groups;

R^{e} is selected from the group consisting of halogen, OH, C₁₋₁₀alkyl, O—C₁₋₁₀alkyl, C₁₋₁₀haloalkyl, cyano, C₃₋₈cycloalkyl, C₆₋₁₀aryl, and NR^{g}R^{h};

R^{f} is selected from the group consisting of hydrogen, unsubstituted or substituted C₁₋₁₀alkyl, unsubstituted or substituted C₁₋₁₀haloalkyl, unsubstituted or substituted C₃₋₈cycloalkyl; wherein each R^{f} may be optionally substituted with one to five R^{e} groups; and

R^{g} and R^{h} are each independently selected from the group consisting of C₁₋₁₀alkyl, C₃₋₈cycloalkyl, and C₆₋₁₀aryl,

or a pharmaceutically acceptable salt thereof.

In embodiments, R² is

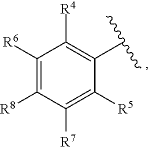

wherein

R⁴, R⁵, R⁶, and R⁷ are each independently selected from the group consisting of hydrogen, halogen, C₁₋₁₀alkyl, C₁₋₁₀haloalkyl, O—C₁₋₁₀alkyl, and NR^{a}R^{b}, wherein R^{a} and R^{b} are each independently selected from the group consisting of C₁₋₁₀alkyl, C₃₋₈cycloalkyl, and C₆₋₁₀aryl;

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted O—C₁₋₁₀haloalkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, unsubstituted or substituted C₅₋₁₀heteroaryl; and unsubstituted or substituted NRCR^{d}, wherein R^{c} and R^{d} are each independently selected from the group consisting of unsubstituted or substituted C₁₋₁₀alkyl, unsubstituted or substituted C₃₋₈cycloalkyl, and C₆₋₁₀aryl, wherein each R^{c} and R^{d} may be optionally substituted with one to five R^{e} groups; and wherein each R⁸ may be optionally substituted with one to five R^{e} groups selected from the group consisting of halogen, OH, C₁₋₁₀alkyl, O—C₁₋₁₀alkyl, C₁₋₁₀haloalkyl, O—C₁₋₁₀haloalkyl, cyano, C₃₋₈cycloalkyl, C₆₋₁₀aryl, and NR^{g}R^{h}, wherein R^{g} and R^{h} are each independently selected from the group consisting of C₁₋₁₀alkyl, C₃₋₈cycloalkyl, and C₆₋₁₀aryl.

Also provided herein are compounds of formula (I):

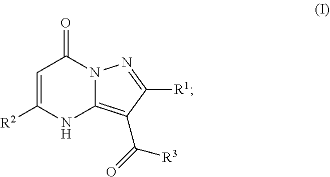

wherein:

R¹ is selected from the group consisting of hydrogen, halogen, C₁₋₁₀alkyl, and C₁₋₁₀haloalkyl;

R² is

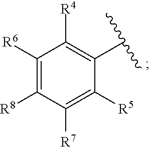

R³ is OR⁹ or NR¹⁰R¹¹;

R⁴, R⁵, R⁶, and R⁷ are each independently selected from the group consisting of hydrogen, halogen, C₁₋₁₀alkyl, C₁₋₁₀haloalkyl, O—C₁₋₁₀alkyl, and NR^{a}R^{b};

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted O—C₁₋₁₀haloalkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, unsubstituted or substituted C₅₋₁₀heteroaryl; and unsubstituted or substituted NR^{c}R^{d}; wherein each R⁸ may be optionally substituted with one to five R^{e} groups;

R⁹ is selected from the group consisting of unsubstituted or substituted C₁₋₁₀alkyl, unsubstituted or substituted C₃₋₈cycloalkyl, unsubstituted or substituted C₆₋₁₀aryl, and unsubstituted or substituted C₅₋₁₀heteroaryl; wherein each R⁹ may be optionally substituted with one to five R^{e} groups;

R¹⁰ and R¹¹ are each independently selected from the group consisting of hydrogen, unsubstituted or substituted C₁₋₁₀alkyl, unsubstituted or substituted C₃₋₁₀cycloalkyl, unsubstituted or substituted C₆₋₁₀aryl, unsubstituted or substituted C₅₋₁₀heteroaryl, unsubstituted or substituted CR^{f}₂—C₆₋₁₀aryl, and R¹⁰ and R¹¹ cyclized to form a unsubstituted or substituted ring having 3-8 ring members; wherein each R¹⁰, R¹¹, and the ring having 3-8 ring members may be optionally substituted with one to five R^{e} groups;

R^{a} and R^{b} are each independently selected from the group consisting of C₁₋₁₀alkyl, C₃₋₈cycloalkyl, and C₆₋₁₀aryl;

R^{c} and R^{d} are each independently selected from the group consisting of unsubstituted or substituted C₁₋₁₀alkyl, unsubstituted or substituted C₃₋₈cycloalkyl, and C₆₋₁₀aryl; wherein each R^{c} and R^{d} may be optionally substituted with one to five R^{e} groups;

R^{e} is selected from the group consisting of halogen, OH, C₁₋₁₀alkyl, O—C₁₋₁₀alkyl, C₁₋₁₀haloalkyl, O—C₁₋₁₀haloalkyl, cyano, C₃₋₈cycloalkyl, C₆₋₁₀aryl, and NR^{g}R^{h};

R^{f} is selected from the group consisting of hydrogen, unsubstituted or substituted C₁₋₁₀alkyl, unsubstituted or substituted C₁₋₁₀haloalkyl, unsubstituted or substituted C₃₋₈cycloalkyl; wherein each R^{f} may be optionally substituted with one to five R^{e} groups; and

R^{g} and R^{h} are each independently selected from the group consisting of C₁₋₁₀alkyl, C₃₋₈cycloalkyl, and C₆₋₁₀aryl,

or a pharmaceutically acceptable salt thereof.

In some embodiments, the compounds of formula (I) are isotopically-labeled by having one or more atoms therein replaced by an atom having a different atomic mass or mass number. Such isotopically-labeled (e.g., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

In another embodiment, the disclosure provides for a pharmaceutical composition, comprising a therapeutically effective amount of a compound according to formula (I) and a pharmaceutically acceptable carrier, diluent and/or excipient.

In addition to salt forms, the present disclosure provides compounds which are in a prodrug form. As used herein the term “prodrug” refers to those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Prodrugs of the disclosure may include phosphates, phosphate esters, alkyl phosphates, alkyl phosphate esters, acyl ethers, or other prodrug moieties as discussed below. In some embodiments, the prodrug moiety is:

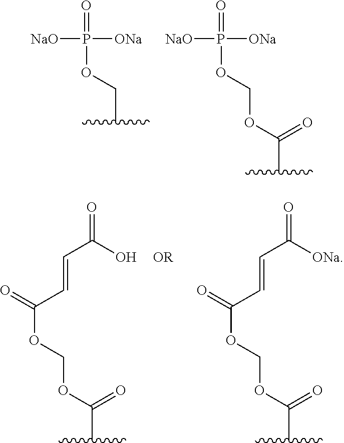

Additional types of prodrugs are also encompassed. For example, where an amino acid residue, or a polypeptide chain of two or more (e.g., two, three or four) amino acid residues, is covalently joined through an amide or ester bond to a free amino, hydroxy or carboxylic acid group of a compound of the present disclosure. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methylalanine, para-benzoylphenylalanine, phenylglycine, propargylglycine, sarcosine, methionine sulfone and tert-butylglycine.

Additional types of prodrugs are also encompassed. For instance, a free carboxyl group of a compound of the disclosure can be derivatized as an amide or alkyl ester. As another example, compounds of this disclosure comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxyl groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery R^{e} views, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Additionally, the present disclosure provides for metabolites of compounds of the disclosure. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound.

Metabolite products typically are identified by preparing a radiolabeled (e.g., ¹⁴C or ³H) isotope of a compound of the disclosure, administering it parenterally in a detectable dose (e.g., greater than about 0.5 mg/kg) to an animal such as rat, mouse, guinea pig, monkey, or to human, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the disclosure.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure can exist in multiple crystalline or amorphous forms. In general, all physical forms are equivalent for the uses contemplated by the present disclosure and are intended to be within the scope of the present disclosure.

In certain embodiments, the disclosed compounds are inhibitors of YAP:TEAD protein-protein interaction that bind to TEAD and disrupt the YAP:TEAD protein-protein interaction (“YAP:TEAD inhibitors”). In embodiments, the disclosed compounds are useful for the treatment of cancers, including cancers characterized by solid tumors, through their ability to inhibit YAP:TEAD protein-protein interaction.

Disclosed compounds are provided as shown in the following enumerated embodiments.

### EMBODIMENT 1

Embodiment 1 includes compounds of formula (I):

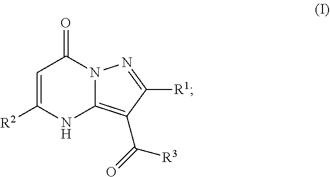

wherein:

R¹ is selected from the group consisting of hydrogen, halogen, C₁₋₁₀alkyl, and C₁₋₁₀haloalkyl;

R² is C₅₋₁₀heteroaryl or

R³ is OR⁹ or NR¹⁰R¹¹;

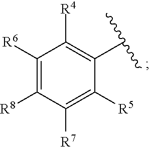

wherein:

when R² is C₅₋₁₀heteroaryl and R³ is NR¹⁰R¹¹, then each of R¹⁰ and R¹¹ is not hydrogen; and

when R³ is OR⁹, then R² is not pyridyl;

R⁴, R⁵, R⁶, and R⁷ are each independently selected from the group consisting of hydrogen, halogen, C₁₋₁₀alkyl, O—C₁₋₁₀alkyl, and NR^{a}R^{b};

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted O—C₁₋₁₀haloalkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, unsubstituted or substituted C₅₋₁₀heteroaryl; and unsubstituted or substituted NR^{c}R^{d}; wherein each R⁸ may be optionally substituted with one to five R^{e} groups;

R⁹ is selected from the group consisting of unsubstituted or substituted C₁₋₁₀alkyl, unsubstituted or substituted C₃₋₈cycloalkyl, unsubstituted or substituted C₆₋₁₀aryl, and unsubstituted or substituted C₅₋₁₀heteroaryl; wherein each R⁹ may be optionally substituted with one to five R^{e} groups;

R¹⁰ and R¹¹ are each independently selected from the group consisting of hydrogen, unsubstituted or substituted C₁₋₁₀alkyl, unsubstituted or substituted C₃₋₁₀cycloalkyl, unsubstituted or substituted C₆₋₁₀aryl, unsubstituted or substituted C₅₋₁₀heteroaryl, unsubstituted or substituted CR^{f}₂—C₆₋₁₀aryl, and R¹⁰ and R¹¹ cyclized to form a unsubstituted or substituted ring having 3-8 ring members; wherein each R¹⁰, R¹¹;

and the ring having 3-8 ring members may be optionally substituted with one to five R^{e} groups;

R^{a} and R^{b} are each independently selected from the group consisting of C₁₋₁₀alkyl, C₃₋₈cycloalkyl, and C₆₋₁₀aryl;

R^{c} and R^{d} are each independently selected from the group consisting of unsubstituted or substituted C₁₋₁₀alkyl, unsubstituted or substituted C₃₋₈cycloalkyl, and C₆₋₁₀aryl; wherein each R^{c} and R^{d} may be optionally substituted with one to five R^{e} groups;

R^{e} is selected from the group consisting of halogen, OH, C₁₋₁₀alkyl, O—C₁₋₁₀alkyl, C₁₋₁₀haloalkyl, O—C₁₋₁₀haloalkyl, cyano, C₃₋₈cycloalkyl, C₆₋₁₀aryl, and NR^{g}R^{h};

R^{f} is selected from the group consisting of hydrogen, unsubstituted or substituted C₁₋₁₀alkyl, unsubstituted or substituted C₁₋₁₀haloalkyl, unsubstituted or substituted C₃₋₈cycloalkyl; wherein each R^{f} may be optionally substituted with one to five R^{e} groups; and

R^{g} and R^{h} are each independently selected from the group consisting of C₁₋₁₀alkyl, C₃₋₈cycloalkyl, and C₆₋₁₀aryl, or a pharmaceutically acceptable salt thereof.

### EMBODIMENT 2

In certain variations of Embodiment 1,

R¹ is selected from the group consisting of hydrogen, halogen, and C1-10alkyl;

R² is

R³ is OR⁹ or NR¹⁰R¹¹;

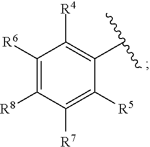

R⁴, R⁵, R⁶, and R⁷ are each independently selected from the group consisting of hydrogen and C₁₋₁₀alkyl;

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, and unsubstituted or substituted NR^{c}R^{d}; wherein each R⁸ may be optionally substituted with one to five R^{e} groups;

R⁹ is unsubstituted or substituted C₁₋₁₀alkyl; wherein each C₁₋₁₀alkyl may be optionally substituted with one to five R^{e} groups;

R¹⁰ and R¹¹ are each independently selected from the group consisting of hydrogen, unsubstituted or substituted C₁₋₁₀alkyl, and R¹⁰ and R¹¹ cyclized to form a unsubstituted or substituted ring having 3-8 ring members; wherein each R¹⁰, R¹¹, and the ring having 3-8 ring members may be optionally substituted with one to five R^{e} groups;

R^{c} and R^{d} are each independently unsubstituted or substituted C₁₋₁₀alkyl; wherein each R^{c} and R^{d} may be optionally substituted with one to five R^{e} groups; and

R^{e} is selected from the group consisting of halogen, OH, C₁₋₁₀alkyl, cyano, and C₃₋₈cycloalkyl;

or a pharmaceutically acceptable salt thereof.

### EMBODIMENT 3

In certain variations of Embodiments 1 or 2, R³ is OR⁹, or a pharmaceutically acceptable salt thereof.

### EMBODIMENT 4

In certain variations of Embodiment 3, R⁹ is an unsubstituted ethyl, or a pharmaceutically acceptable salt thereof.

### EMBODIMENT 5

In certain variations of Embodiments 3 or 4, R² is C₅₋₁₀heteroaryl, or a pharmaceutically acceptable salt thereof.

### EMBODIMENT 6

In certain variations of Embodiments 3 or 4, R² is selected from the group consisting of:

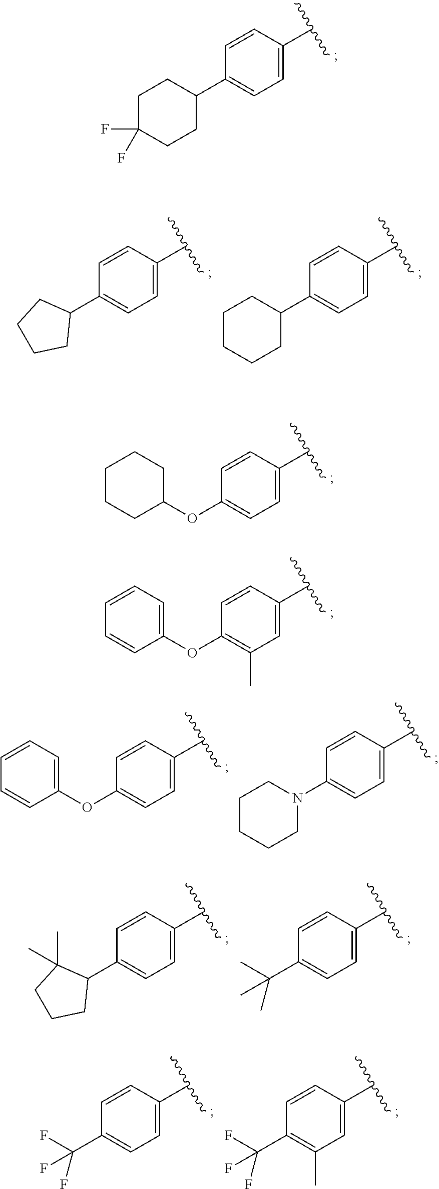

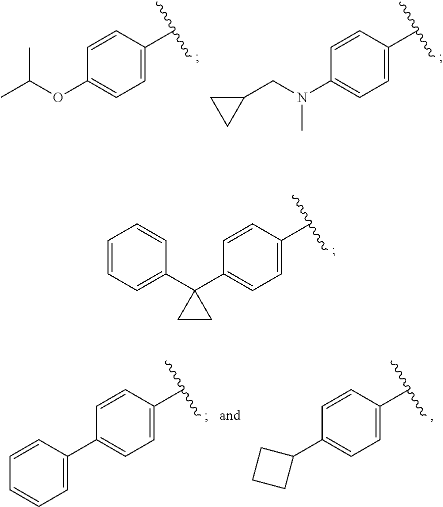 [SMILES: *c1ccc(OC(C)C)cc1 ; *c1ccc(N(C)CC2CC2)cc1 ; *c1ccc(C2(c3ccccc3)CC2)cc1 ; *c1ccc(-c2ccccc2)cc1 ; *c1ccc(C2CCC2)cc1]

or a pharmaceutically acceptable salt thereof.

### EMBODIMENT 7

In certain variations of Embodiments 3 or 4, R² is:

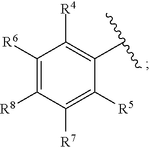

or a pharmaceutically acceptable salt thereof.

### EMBODIMENT 8

In certain variations of Embodiment 7, R⁴, R⁵, R⁶, and R⁷ are each hydrogen; and R⁸ is selected from the group consisting of:

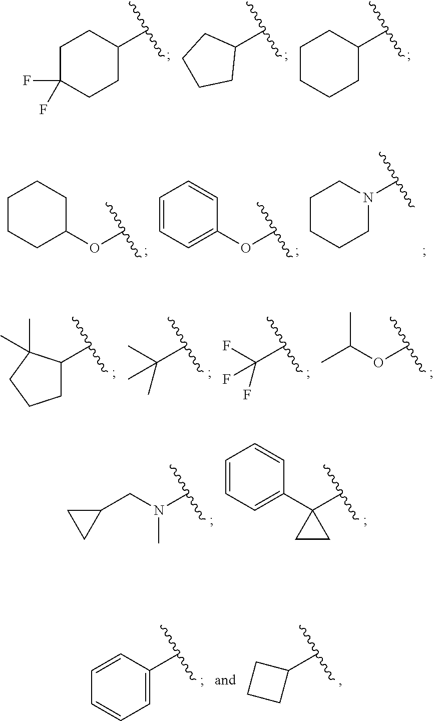

or a pharmaceutically acceptable salt thereof.

### EMBODIMENT 9

In certain variations of Embodiment 7, R⁸ is selected from the group consisting of:

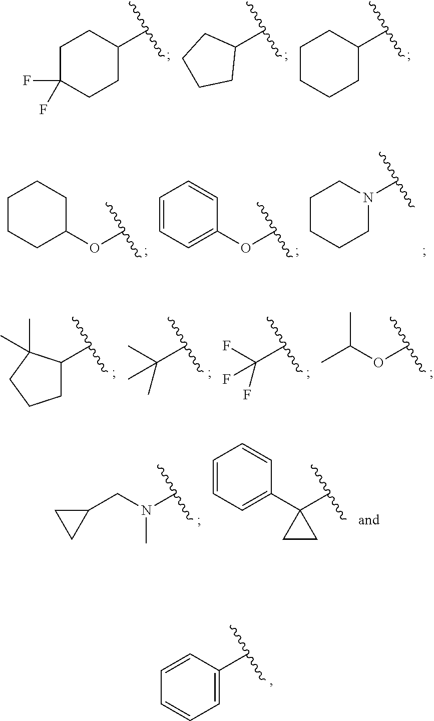

or a pharmaceutically acceptable salt thereof.

### EMBODIMENT 10

In certain variations of Embodiments 1 or 2, the compounds of formula (I) include compounds of formula (IIc):

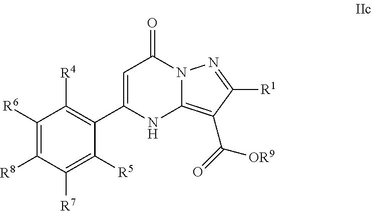

or a pharmaceutically acceptable salt thereof.

### EMBODIMENT 11

In certain variations of Embodiment 10, the compound of formula (IIc) is selected from the group consisting of:

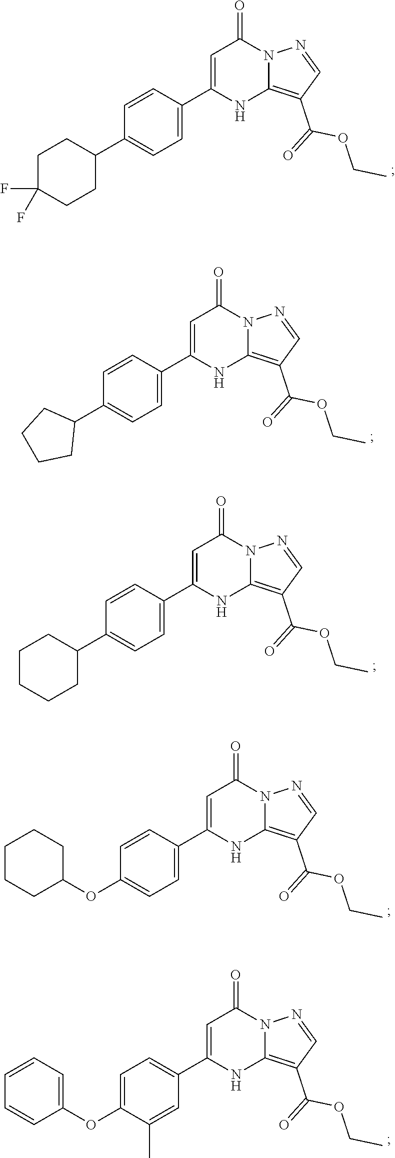 [SMILES: CCOC(=O)c1cnn2c(=O)cc(-c3ccc(C4CCC(F)(F)CC4)cc3)[nH]c12 ; CCOC(=O)c1cnn2c(=O)cc(-c3ccc(C4CCCC4)cc3)[nH]c12 ; CCOC(=O)c1cnn2c(=O)cc(-c3ccc(C4CCCCC4)cc3)[nH]c12 ; CCOC(=O)c1cnn2c(=O)cc(-c3ccc(OC4CCCCC4)cc3)[nH]c12 ; CCOC(=O)c1cnn2c(=O)cc(-c3ccc(Oc4ccccc4)c(C)c3)[nH]c12]

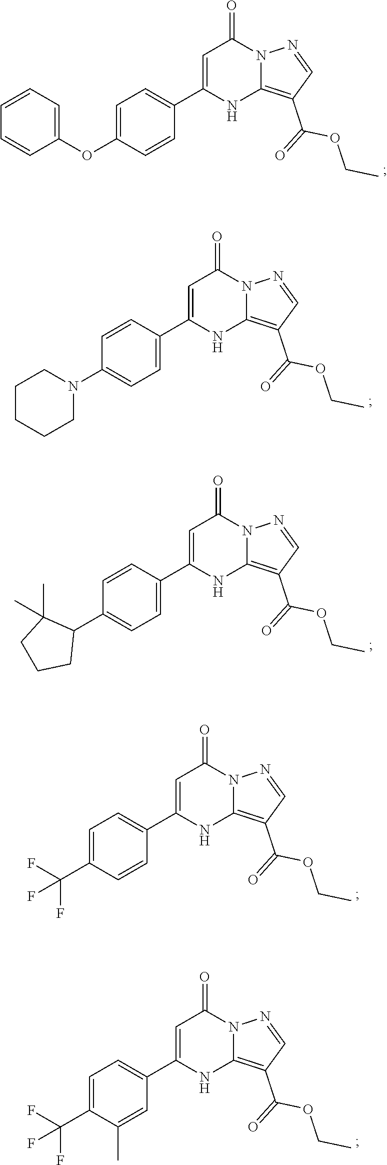

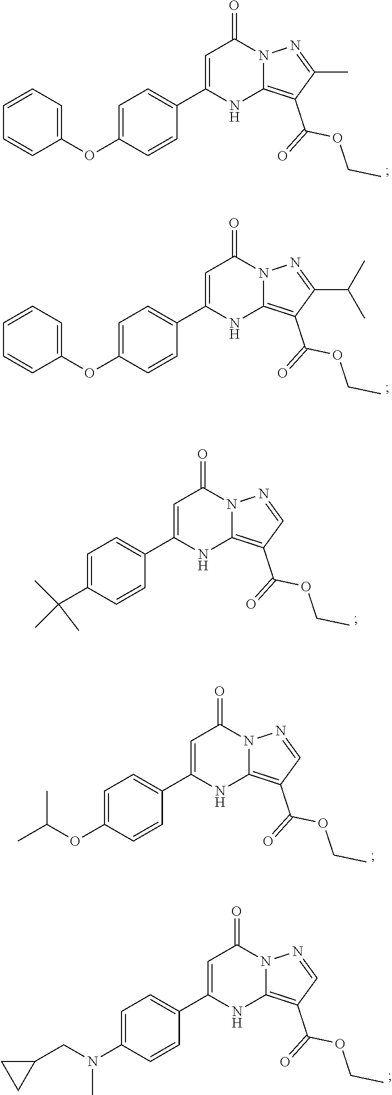

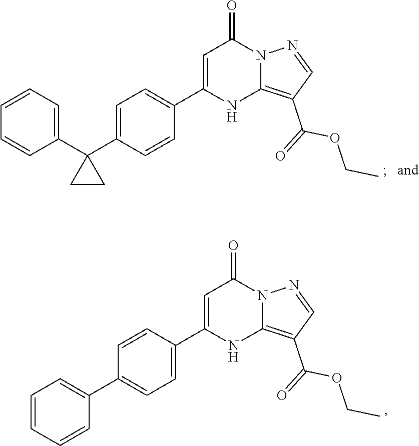 [SMILES: CCOC(=O)c1cnn2c(=O)cc(-c3ccc(C4(c5ccccc5)CC4)cc3)[nH]c12 ; CCOC(=O)c1cnn2c(=O)cc(-c3ccc(-c4ccccc4)cc3)[nH]c12]

or a pharmaceutically acceptable salt thereof.

### EMBODIMENT 12

In certain variations of Embodiments 1 or 2, R3 is NR¹⁰R¹¹, or a pharmaceutically acceptable salt thereof.

### EMBODIMENT 13

In certain variations of Embodiment 12, R¹⁰ and R¹¹ are each independently selected from the group consisting of hydrogen, methyl, ethyl, cyclopropyl, and -ethyl-OH; or R¹⁰ and R¹¹ are cyclized to form a 4 or 5-membered ring with the nitrogen, the ring optionally substituted with one to two R^{e} groups each independently selected from the group consisting of methyl, ethyl, OH, cyano, CH₂—F, CHF₂ and CF₃, or a pharmaceutically acceptable salt thereof.

### EMBODIMENT 14

In certain variations of Embodiments 12 or 13, R² is C₅₋₁₀heteroaryl, or a pharmaceutically acceptable salt thereof.

### EMBODIMENT 15

In certain variations of Embodiments 12 or 13, R² is selected from the group consisting of:

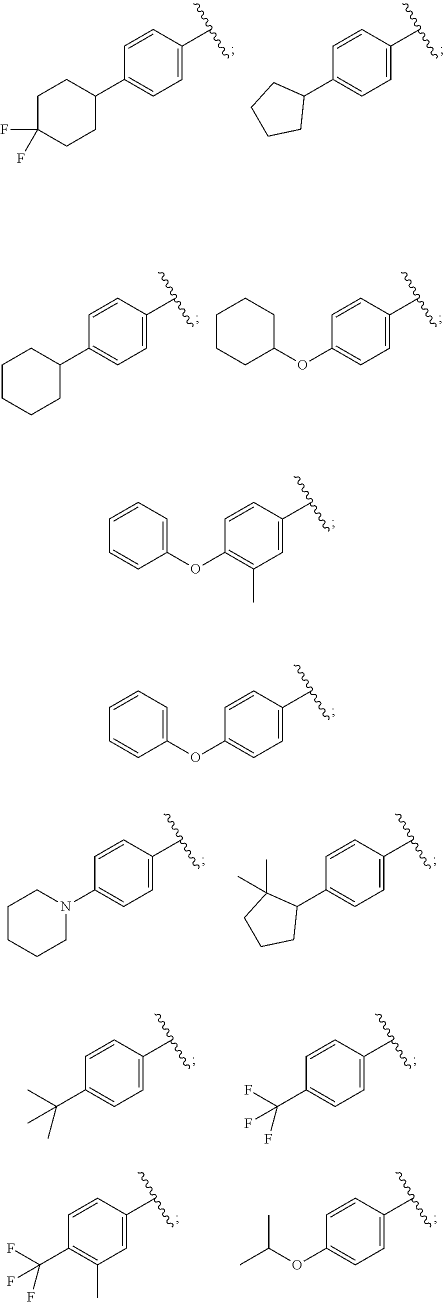

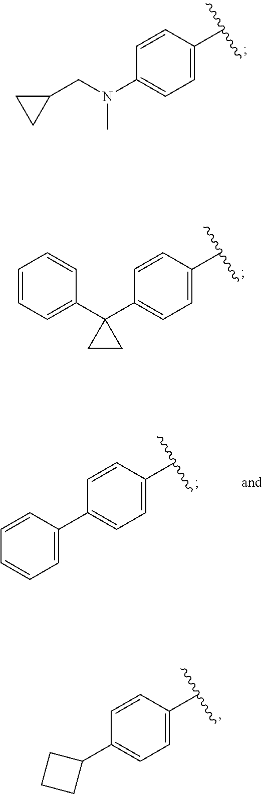 [SMILES: *c1ccc(N(C)CC2CC2)cc1 ; *c1ccc(C2(c3ccccc3)CC2)cc1 ; *c1ccc(-c2ccccc2)cc1 ; *c1ccc(C2CCC2)cc1]

or a pharmaceutically acceptable salt thereof.

### EMBODIMENT 16

In certain variations of Embodiments 12 or 13, R² is selected from the group consisting of:

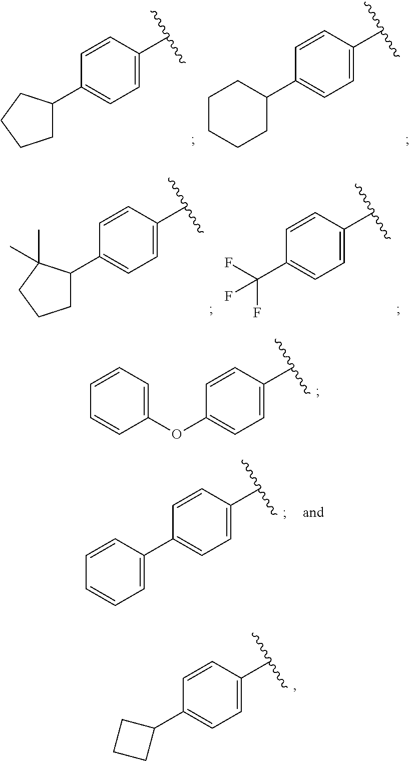

or a pharmaceutically acceptable salt thereof.

### EMBODIMENT 17

In certain variations of Embodiments 12 or 13, R² is:

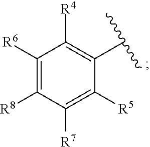

or a pharmaceutically acceptable salt thereof.

### EMBODIMENT 18

In certain variations of Embodiment 17, R⁴, R⁵, R⁶, and R⁷ are each hydrogen; and R⁸ is selected from the group consisting of:

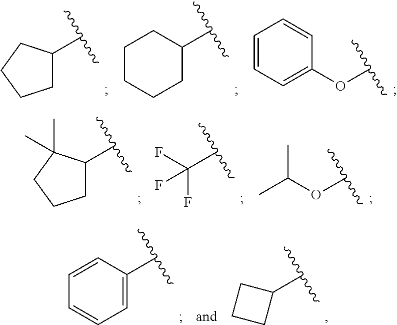

or a pharmaceutically acceptable salt thereof.

### EMBODIMENT 19

In certain variations of Embodiments 1 or 2, the compounds of formula (I) include compounds of formula (He):

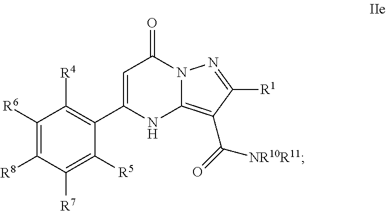

or a pharmaceutically acceptable salt thereof.

### EMBODIMENT 20

In certain variations of Embodiment 19, the compound of formula (IIe) is selected from the group consisting of:

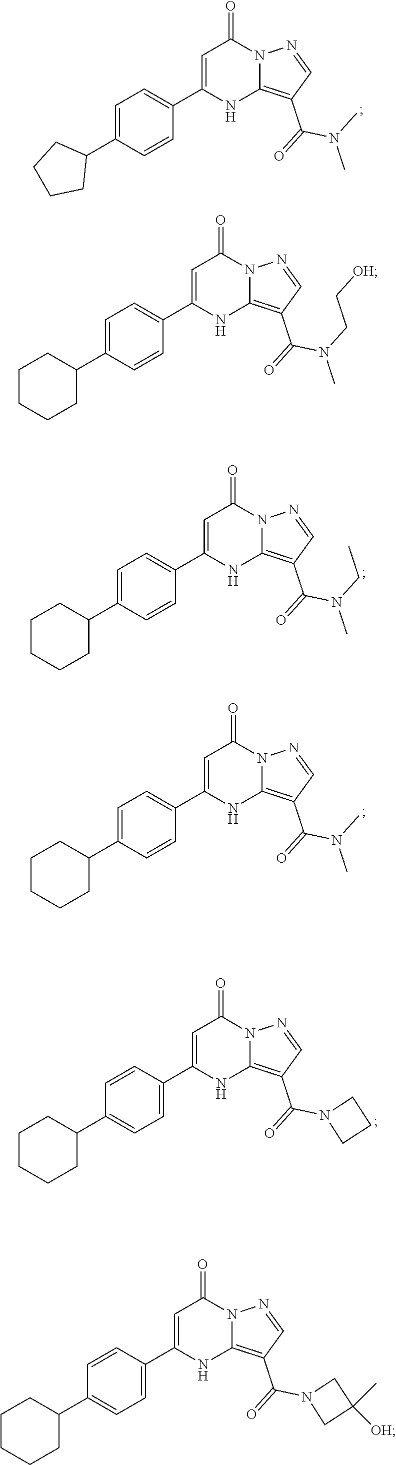 [SMILES: CN(C)C(=O)c1cnn2c(=O)cc(-c3ccc(C4CCCC4)cc3)[nH]c12 ; CN(CCO)C(=O)c1cnn2c(=O)cc(-c3ccc(C4CCCCC4)cc3)[nH]c12 ; CCN(C)C(=O)c1cnn2c(=O)cc(-c3ccc(C4CCCCC4)cc3)[nH]c12 ; CN(C)C(=O)c1cnn2c(=O)cc(-c3ccc(C4CCCCC4)cc3)[nH]c12 ; O=C(c1cnn2c(=O)cc(-c3ccc(C4CCCCC4)cc3)[nH]c12)N1CCC1 ; CC1(O)CN(C(=O)c2cnn3c(=O)cc(-c4ccc(C5CCCCC5)cc4)[nH]c23)C1]

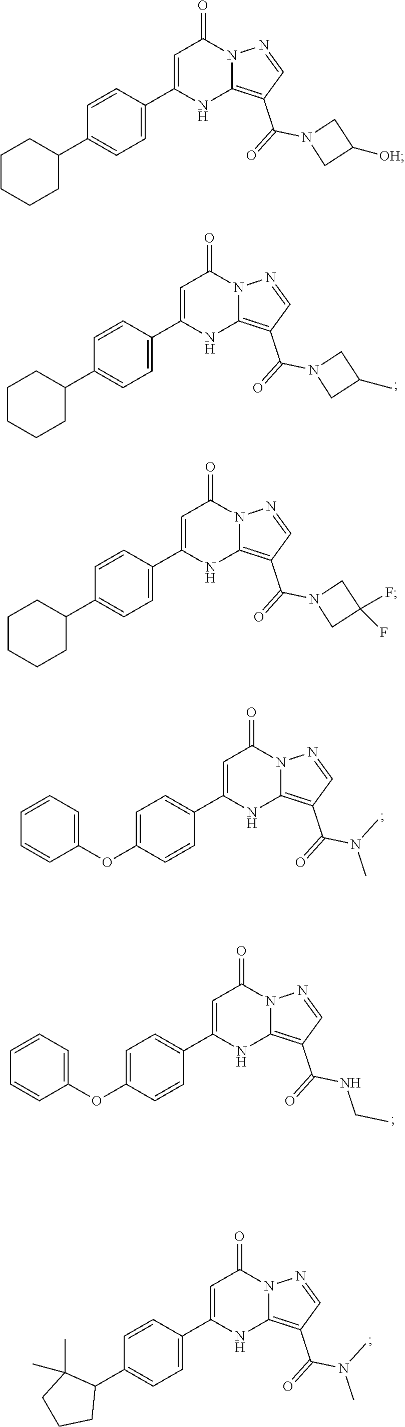

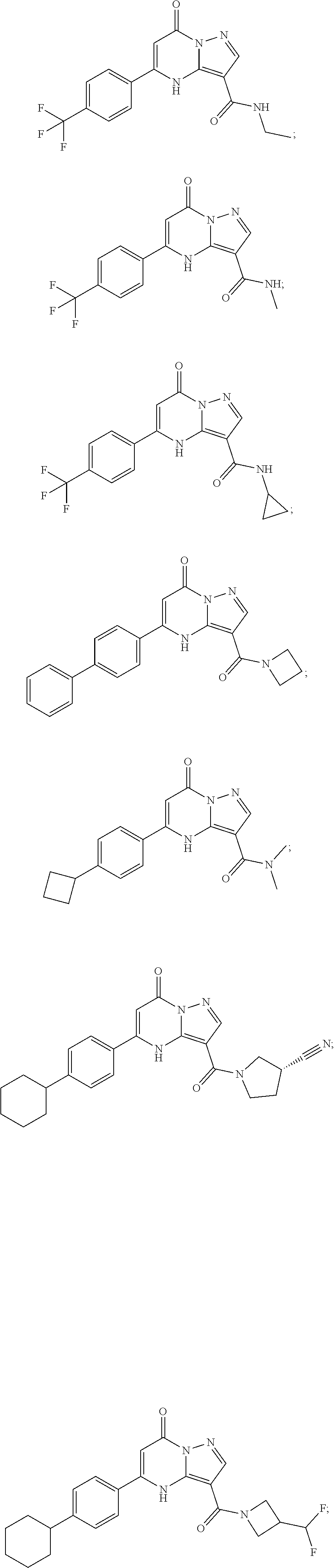

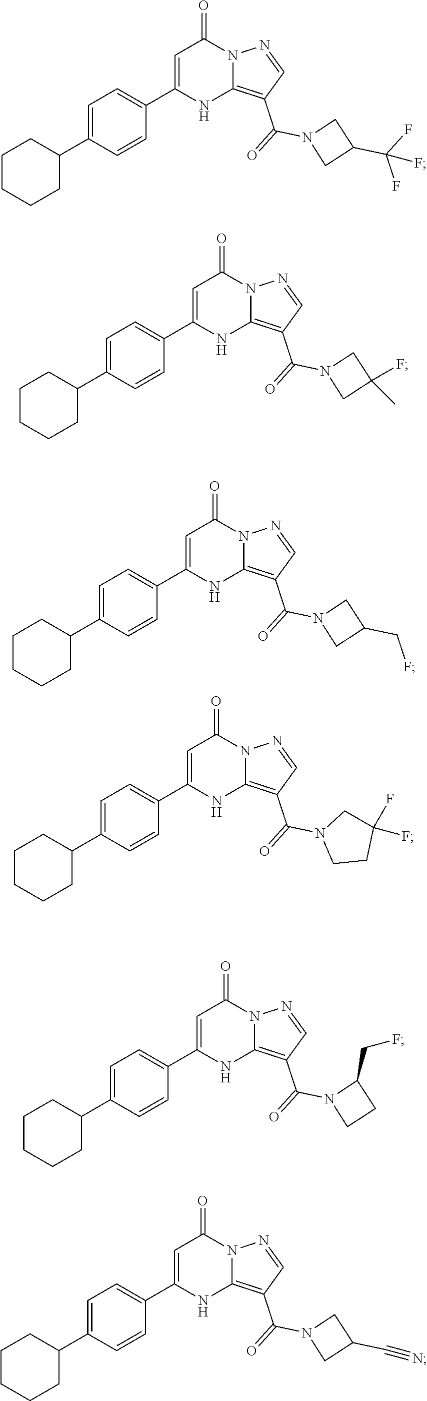 [SMILES: O=C(c1cnn2c(=O)cc(-c3ccc(C4CCCCC4)cc3)[nH]c12)N1CC(C(F)(F)F)C1 ; CC1(F)CN(C(=O)c2cnn3c(=O)cc(-c4ccc(C5CCCCC5)cc4)[nH]c23)C1 ; O=C(c1cnn2c(=O)cc(-c3ccc(C4CCCCC4)cc3)[nH]c12)N1CC(CF)C1 ; O=C(c1cnn2c(=O)cc(-c3ccc(C4CCCCC4)cc3)[nH]c12)N1CCC(F)(F)C1 ; O=C(c1cnn2c(=O)cc(-c3ccc(C4CCCCC4)cc3)[nH]c12)N1CC[C@@H]1CF ; N#CC1CN(C(=O)c2cnn3c(=O)cc(-c4ccc(C5CCCCC5)cc4)[nH]c23)C1]

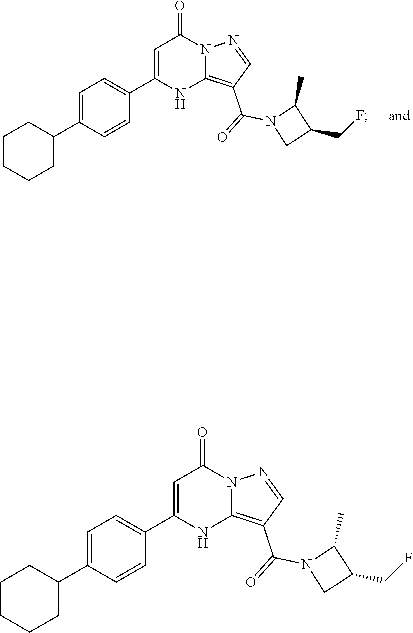 [SMILES: C[C@H]1[C@@H](CF)CN1C(=O)c1cnn2c(=O)cc(-c3ccc(C4CCCCC4)cc3)[nH]c12 ; "and" ; C[C@@H]1[C@H](CF)CN1C(=O)c1cnn2c(=O)cc(-c3ccc(C4CCCCC4)cc3)[nH]c12]

or a pharmaceutically acceptable salt thereof.

### EMBODIMENT 21

In certain variations of Embodiments 1, 2 and 12-19, R¹⁰ and R¹¹ are each unsubstituted or substituted C₁₋₁₀alkyl; wherein each R¹⁰ and R¹¹ may be optionally substituted with one to five R^{e} groups, or a pharmaceutically acceptable salt thereof.

### EMBODIMENT 22

In certain variations of Embodiments 1, 2 and 12-19, R¹⁰ and R¹¹ are cyclized to form a unsubstituted or substituted ring having 3-8 ring members; wherein the ring having 3-8 ring members may be optionally substituted with one to five R^{e} groups, or a pharmaceutically acceptable salt thereof.

### EMBODIMENT 23

In certain variations of Embodiments 1, 2 and 12-19, R¹⁰ and R¹¹ are cyclized to form an unsubstituted or substituted ring having 3-8 ring members, or a pharmaceutically acceptable salt thereof.

### EMBODIMENT 24

In certain variations of Embodiments 1, 2 and 12-19, R¹⁰ and R¹¹ are cyclized to form a substituted ring having 3-8 ring members; wherein the ring having 3-8 ring members is substituted with one to five R^{e} groups, or a pharmaceutically acceptable salt thereof.

### EMBODIMENT 25

In certain variations of Embodiments 1, 2, 12-13 and 15-19, R¹⁰ and R¹¹ are each unsubstituted or substituted C₁₋₁₀alkyl; wherein each R¹⁰ and R¹¹ may be optionally substituted with one to two R^{e} groups selected from the group consisting of halogen, OH, C₁₋₁₀alkyl, cyano, and C₃₋₈cycloalkyl; and

R² is:

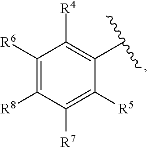

wherein

R⁴, R⁵, R⁶, and R⁷ are each independently selected from the group consisting of hydrogen and C1-3alkyl; and

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, and unsubstituted or substituted NR^{c}R^{d}, wherein R^{c} and R^{d} are each independently selected from the group consisting of unsubstituted or substituted C₁₋₄alkyl, and unsubstituted or substituted C₃₋₆cycloalkyl; and wherein each R⁸ may be optionally substituted with one to two R^{e} groups independently selected from the group consisting of methyl, halogen, C₃₋₄cycloalkyl, and phenyl, or a pharmaceutically acceptable salt thereof.

### EMBODIMENT 26

In certain variations of Embodiments 1, 2, 12-13 and 15-19, R¹⁰ and R¹¹ are cyclized to form a unsubstituted or substituted ring having 3-8 ring members; wherein the ring having 3-8 ring members may be optionally substituted with one to two R^{e} groups selected from the group consisting of halogen, OH, C₁₋₁₀alkyl, cyano, and C₃₋₈cycloalkyl; and

R² is:

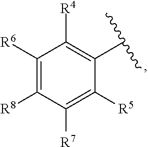

wherein

R⁴, R⁵, R⁶, and R⁷ are each independently selected from the group consisting of hydrogen and C₁₋₃alkyl; and

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, and unsubstituted or substituted NR^{c}R^{d}, wherein R^{c} and R^{d} are each independently selected from the group consisting of unsubstituted or substituted C₁₋₄alkyl, and unsubstituted or substituted C₃₋₆cycloalkyl; and wherein each R⁸ may be optionally substituted with one to two R^{e} groups independently selected from the group consisting of methyl, halogen, C₃₋₄cycloalkyl, and phenyl,

or a pharmaceutically acceptable salt thereof.

### EMBODIMENT 27

In certain variations of Embodiments 1-27, R¹ is hydrogen, halogen, C₁₋₁₀alkyl, and C₁₋₁₀haloalkyl. In other variations of Embodiments 1-27, R¹ is hydrogen. In other variations of Embodiments 1-27, R¹ is halogen. In other variations of Embodiments 1-27, R¹ is fluoro. In other variations of Embodiments 1-27, R¹ is C₁₋₁₀alkyl. In other variations of Embodiments 1-27, R¹ is CH₃.

### EMBODIMENT 28

In embodiments, the compounds of formula (I) include compounds of formula (II):

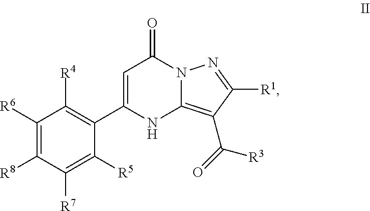

wherein

R¹ is selected from the group consisting of hydrogen, halogen, C₁₋₁₀alkyl, and C₁₋₁₀haloalkyl;

R³ is OR⁹ or NR¹⁰R¹¹, wherein

R⁹ is selected from the group consisting of unsubstituted or substituted C₁₋₁₀alkyl, unsubstituted or substituted C₃₋₈cycloalkyl, unsubstituted or substituted C₆₋₁₀aryl, and unsubstituted or substituted C₅₋₁₀heteroaryl; wherein each R⁹ is optionally substituted with one to five R^{e} groups; and

r¹⁰ and R¹¹ are each independently selected from the group consisting of hydrogen, unsubstituted or substituted C₁₋₁₀alkyl, unsubstituted or substituted C³⁻¹⁰cycloalkyl, unsubstituted or substituted C₆₋₁₀aryl, unsubstituted or substituted C₅₋₁₀heteroaryl, unsubstituted or substituted CR^{f}₂—C₆₋₁₀aryl, and R¹⁰ and R¹¹ cyclized with N to form a unsubstituted or substituted ring having 3-8 ring members; wherein each R¹⁰, R¹¹, and the ring having 3-8 ring members is optionally substituted with one to five R^{e} groups, wherein

R^{f} is selected from the group consisting of hydrogen, unsubstituted or substituted C₁₋₁₀alkyl, unsubstituted or substituted C₁₋₁₀haloalkyl, unsubstituted or substituted C₃₋₈cycloalkyl; wherein each R^{f} is optionally substituted with one to five R^{e} groups;

R⁴, R⁵, R⁶, and R⁷ are each independently selected from the group consisting of hydrogen, halogen, C₁₋₁₀alkyl, C₁₋₁₀haloalkyl, O—C₁₋₁₀alkyl, and NR^{a}R^{b}, wherein

R^{a} and R^{b} are each independently selected from the group consisting of C₁₋₁₀alkyl, C₃₋₈cycloalkyl, and C₆₋₁₀aryl; and

R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted O—C₁₋₁₀haloalkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, unsubstituted or substituted C₅₋₁₀heteroaryl; and unsubstituted or substituted NR^{c}R^{d}; wherein each R⁸ is optionally substituted with one to five R^{e} groups, wherein

R^{c} and R^{d} are each independently selected from the group consisting of unsubstituted or substituted C₁₋₁₀alkyl, unsubstituted or substituted C₃₋₈cycloalkyl, and C₆₋₁₀aryl; wherein each R^{c} and R^{d} is optionally substituted with one to five R^{e} groups;

R^{e} is selected from the group consisting of halogen, OH, C₁₋₁₀alkyl, O—C₁₋₁₀alkyl, C₁₋₁₀haloalkyl, O—C₁₋₁₀haloalkyl, C₃₋₈cycloalkyl, C₆₋₁₀aryl, and NR^{g}R^{h}, wherein R^{g} and R^{h} are each independently selected from the group consisting of C₁₋₁₀alkyl, C₃₋₈cycloalkyl, and C₆₋₁₀aryl, or a pharmaceutically acceptable salt thereof.

### EMBODIMENT 29

In certain variations of Embodiment 28,

R¹ is hydrogen or C₁₋₃alkyl;

R³ is OR⁹ or NR¹⁰R¹¹, wherein

R⁹ is C₁₋₂alkyl; and

R¹⁰ and R¹¹ are each independently selected from the group consisting of hydrogen, unsubstituted or substituted C₁₋₂alkyl, and C₃₋₆cycloalkyl, wherein R¹⁰ andR¹¹ are each independently optionally substituted with one or two R^{e} substituents selected from the group consisting of OH and C₁₋₃alkyl, or

R¹⁰ and R¹¹ are cyclized with N to form an unsubstituted or substituted ring having 4 ring members;

wherein said 4-membered ring is optionally substituted with one or two R^{e} substituents selected from the group consisting of halogen, OH and C₁₋₃alkyl;

R⁴, R⁵, R⁶, and R⁷ are each hydrogen or C₁₋₂alkyl; and

R⁸ is selected from the group consisting of unsubstituted or substituted C₁₋₄alkyl, unsubstituted or substituted O—C₁₋₃alkyl, unsubstituted or substituted C₆aryl, unsubstituted or substituted O—C₆aryl, unsubstituted or substituted C₃₋₆cycloalkyl, unsubstituted or substituted O—C₃₋₆cycloalkyl, unsubstituted or substituted C₅₋₆heterocycloalkyl, unsubstituted or substituted C₅₋₁₀heteroaryl; and unsubstituted or substituted NR^{c}R^{d}, wherein each R^{c} and R^{d} are independently hydrogen or C₁₋₂alkyl; wherein each R⁸ is optionally substituted with one to five R^{e} substituents selected from the group consisting of halogen, C₁₋₂alkyl, C₃cycloalkyl, and C₆aryl,

or a pharmaceutically acceptable salt thereof.

### EMBODIMENT 30

In certain variations of Embodiments 28-29,

R¹ is hydrogen;

R³ is OR⁹ or NR¹⁰R¹¹, wherein

R⁹ is C₁₋₂alkyl; and

R¹⁰ and R¹¹ are each independently selected from the group consisting of hydrogen, unsubstituted or substituted C₁₋₂alkyl, and C₃₋₆cycloalkyl, wherein R¹⁰ and R¹¹ are each independently optionally substituted with one or two R^{e} substituents selected from the group consisting of OH and C₁₋₃alkyl, or

R¹⁰ and R¹¹ are cyclized with N to form an unsubstituted or substituted ring having 4 ring members; wherein said 4-membered ring is optionally substituted with one or two R^{e} substituents selected from the group consisting of halogen, OH and C₁₋₃alkyl;

R⁴, R⁵, R⁶, and R⁷ are each hydrogen or C₁₋₂alkyl; and

R⁸ is cyclohexyl optionally substituted with one or two halogens,

or a pharmaceutically acceptable salt thereof.

### EMBODIMENT 31

In certain variations of Embodiments 28-29,

R¹ is hydrogen;

R³ is OR⁹ or NR¹⁰R¹¹, wherein

R⁹ is C₁₋₂alkyl; and

R¹⁰ and R¹¹ are each independently selected from the group consisting of hydrogen, unsubstituted or substituted C₁₋₂alkyl, and C₃₋₆cycloalkyl, wherein R¹⁰ and R¹¹ are each independently optionally substituted with one or two R^{e} substituents selected from the group consisting of OH and C₁₋₃alkyl, or

R¹⁰ and R¹¹ are cyclized with N to form an unsubstituted or substituted ring having 4 ring members; wherein said 4-membered ring is optionally substituted with one or two W substituents selected from the group consisting of halogen, OH and C₁₋₃alkyl;

R⁴, R⁵, R⁶, and R⁷ are each hydrogen or C₁₋₂alkyl; and

R⁸ is cyclopentyl optionally substituted with one or two C₁₋₂alkyl,

or a pharmaceutically acceptable salt thereof.

### EMBODIMENT 32

In certain variations of Embodiments 28-29,

R¹ is hydrogen;

R³ is OR⁹ or NR¹⁰R¹¹, wherein

R⁹ is C₁₋₂alkyl; and

R¹⁰ and R¹¹ are each independently selected from the group consisting of hydrogen, unsubstituted or substituted C₁₋₂alkyl, and C₃₋₆cycloalkyl, wherein R¹⁰ and R¹¹ are each independently optionally substituted with one or two R^{e} substituents selected from the group consisting of OH and C₁₋₃alkyl, or

R¹⁰ and R¹¹ are cyclized with N to form an unsubstituted or substituted ring having 4 ring members; wherein said 4-membered ring is optionally substituted with one or two R^{e} substituents selected from the group consisting of halogen, OH and C₁₋₃alkyl;

R⁴, R⁵, R⁶, and R⁷ are each hydrogen or C₁₋₂alkyl; and

R⁸ is O-phenyl,

or a pharmaceutically acceptable salt thereof.

### EMBODIMENT 33

In certain variations of Embodiments 28-29,

R¹ is hydrogen;

R³ is OR⁹ or NR¹⁰R¹¹, wherein

R⁹ is C₁₋₂alkyl; and

R¹⁰ and R¹¹ are each independently selected from the group consisting of hydrogen, unsubstituted or substituted C₁₋₂alkyl, and C₃₋₆cycloalkyl, wherein R¹⁰ and R¹¹ are each independently optionally substituted with one or two R^{e} substituents selected from the group consisting of OH and C₁₋₃alkyl, or

R¹⁰ and R¹¹ are cyclized with N to form an unsubstituted or substituted ring having 4 ring members; wherein said 4-membered ring is optionally substituted with one or two R^{e} substituents selected from the group consisting of halogen, OH and C₁₋₃alkyl;

R⁴, R⁵, R⁶, and R⁷ are each hydrogen or C₁₋₂alkyl; and

R⁸ is C₁₋₄alkyl optionally substituted with one to three halogen, C₁₋₂alkyl, or a pharmaceutically acceptable salt thereof.

### EMBODIMENT 34

In certain variations of Embodiments 28-29,

R¹ is hydrogen;

R³ is OR⁹ or NR¹⁰R¹¹, wherein

R⁹ is C₁₋₂alkyl; and

R¹⁰ and R¹¹ are each independently selected from the group consisting of hydrogen, unsubstituted or substituted C₁₋₂alkyl, and C₃₋₆cycloalkyl, wherein R¹⁰ and R¹¹ are each independently optionally substituted with one or two R^{e} substituents selected from the group consisting of OH and C₁₋₃alkyl, or

R¹⁰ and R¹¹ are cyclized with N to form an unsubstituted or substituted ring having 4 ring members; wherein said 4-membered ring is optionally substituted with one or two R^{e} substituents selected from the group consisting of halogen, OH and C₁₋₃alkyl;

R⁴, R⁵, R⁶, and R⁷ are each hydrogen or C₁₋₂alkyl; and

R⁸ is phenyl,

or a pharmaceutically acceptable salt thereof.

### EMBODIMENT 35

In certain variations of Embodiments 28-29,

R¹ is hydrogen;

R³ is OR⁹ wherein R⁹ is C₁₋₂alkyl

R⁴, R⁵, R⁶, and R⁷ are each hydrogen or C₁₋₂alkyl; and

R⁸ is selected from the group consisting of unsubstituted or substituted C₁₋₄alkyl, unsubstituted or substituted O—C₁₋₃alkyl, unsubstituted or substituted C₆aryl, unsubstituted or substituted O—C₆aryl, unsubstituted or substituted C₃₋₆cycloalkyl, unsubstituted or substituted O—C₃₋₆cycloalkyl, unsubstituted or substituted C₅₋₆heterocycloalkyl, unsubstituted or substituted C₅₋₁₀heteroaryl; and unsubstituted or substituted NR^{c}R^{d}, wherein each R^{c} and R^{d} are independently hydrogen or C₁₋₂alkyl; wherein each R⁸ is optionally substituted with one to five R^{e} substituents selected from the group consisting of halogen, C₁₋₂alkyl, C₃cycloalkyl, and C₆aryl,

or a pharmaceutically acceptable salt thereof.

### EMBODIMENT 36

In certain variations of Embodiments 28-29,

R¹ is hydrogen;

R³ is NR¹⁰R¹¹, wherein

R¹⁰ and R¹¹ are each independently selected from the group consisting of hydrogen, unsubstituted or substituted C₁₋₂alkyl, and C₃₋₆cycloalkyl, wherein R¹⁰ and R¹¹ are each independently optionally substituted with one or two R^{e} substituents selected from the group consisting of OH and C₁₋₃alkyl, or

R¹⁰ and R¹¹ are cyclized with N to form an unsubstituted or substituted ring having 4 ring members; wherein said 4-membered ring is optionally substituted with one or two R^{e} substituents selected from the group consisting of halogen, OH and C₁₋₃alkyl;

R⁴, R⁵, R⁶, and R⁷ are each hydrogen or C₁₋₂alkyl; and

R⁸ is selected from the group consisting of unsubstituted or substituted C₁₋₄alkyl, unsubstituted or substituted O—C₁₋₃alkyl, unsubstituted or substituted C₆aryl, unsubstituted or substituted O—C₆aryl, unsubstituted or substituted C₃₋₆cycloalkyl, unsubstituted or substituted O—C₃₋₆cycloalkyl, unsubstituted or substituted C₅₋₆heterocycloalkyl, unsubstituted or substituted C₅₋₁₀heteroaryl; and unsubstituted or substituted NR^{c}R^{d}, wherein each R^{c} and R^{d} are independently hydrogen or C₁₋₂alkyl; wherein each R⁸ is optionally substituted with one to five R^{e} substituents selected from the group consisting of halogen, C₁₋₂alkyl, C₃cycloalkyl, and C₆aryl,

or a pharmaceutically acceptable salt thereof.

### EMBODIMENT 37

In certain variations of Embodiments 28-29,

R¹ is hydrogen;

R³ is NR¹⁰R¹¹, wherein R¹⁰ and R¹¹ are cyclized with N to form an unsubstituted or substituted ring having 3-8 ring members each optionally substituted with one or two R^{e} groups selected from the group consisting of halogen, OH, C₁₋₁₀alkyl, O—C₁₋₁₀alkyl, C₁₋₁₀haloalkyl, O—C₁₋₁₀haloalkyl, cyano, C₃₋₈cycloalkyl, C₆₋₁₀aryl, and NR^{g}R^{h};

R⁴, R⁵, R⁶, and R⁷ are each hydrogen; and

R⁸ is C₃₋₈cycloalkyl optionally substituted with one to five R^{e} groups selected from the group consisting of halogen, OH, C₁₋₁₀alkyl, O—C₁₋₁₀alkyl, C₁₋₁₀haloalkyl, cyano, C₃₋₈cycloalkyl, C₆₋₁₀aryl, and NR^{g}R^{h}, wherein R^{g} and R^{h} are each independently selected from the group consisting of C₁₋₁₀alkyl, C₃₋₈cycloalkyl, and C₁₋₁₀aryl,

or a pharmaceutically acceptable salt thereof.

### EMBODIMENT 38

In certain variations of Embodiment 37,

R¹⁰ and R¹¹ are cyclized with N to form a substituted ring having 4-5 ring members wherein at least one member is substituted with one or two R^{e} groups selected from the group consisting of halogen, C₁₋₂alkyl, C₁₋₂haloalkyl and cyano; and

R⁸ is cyclohexyl,

or a pharmaceutically acceptable salt thereof.

### EMBODIMENT 39

In certain variations of Embodiments 28 or 29, R³ is OR⁹, or a pharmaceutically acceptable salt thereof.

### EMBODIMENT 40

In certain variations of Embodiment 39, R⁹ is an unsubstituted ethyl, or a pharmaceutically acceptable salt thereof.

### EMBODIMENT 41

In certain variations of Embodiments 39 or 40, R⁴, R⁵, R⁶, and R⁷ are each hydrogen; and R⁸ selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, and unsubstituted or substituted NR^{c}R^{d}, wherein R^{c} and R^{d} are each independently selected from the group consisting of unsubstituted or substituted C₁₋₄alkyl, and unsubstituted or substituted C₃₋₆cycloalkyl; and wherein each R⁸ may be optionally substituted with one to two R^{e} groups independently selected from the group consisting of methyl, halogen, C₃₋₄cycloalkyl, and phenyl, or a pharmaceutically acceptable salt thereof.

### EMBODIMENT 42

In certain variations of Embodiments 39 or 40, R⁴, R⁵, R⁶, and R⁷ are each hydrogen; and R⁸ is selected from the group consisting of:

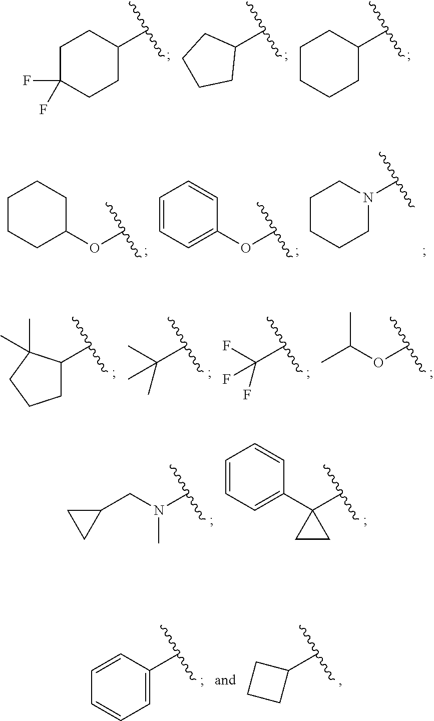

or a pharmaceutically acceptable salt thereof.

### EMBODIMENT 43

In certain variations of Embodiment 41, R⁸ is selected from the group consisting of:

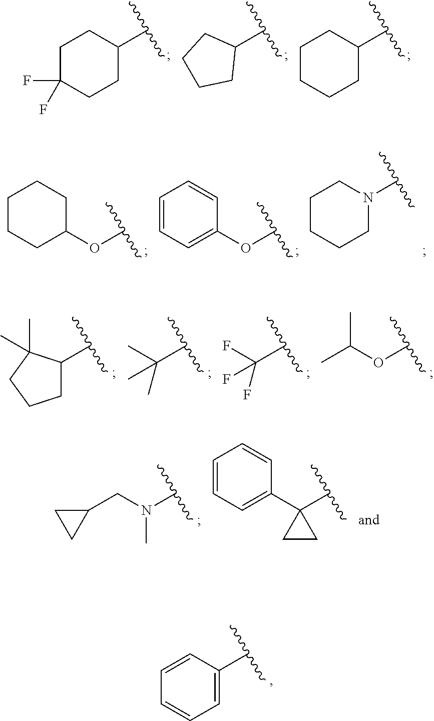

or a pharmaceutically acceptable salt thereof.

### EMBODIMENT 44

In certain variations of Embodiments 28 or 29, R³ is NR¹⁰R¹¹ or a pharmaceutically acceptable salt thereof.

### EMBODIMENT 45

In certain variations of Embodiment 44, R¹⁰ and R¹¹ are each independently selected from the group consisting of hydrogen, methyl, ethyl, cyclopropyl, and -ethyl-OH; or R¹⁰ and R¹¹ are cyclized to form a 4 or 5-membered ring with the nitrogen, the ring optionally substituted with one to two R^{e} groups each independently selected from the group consisting of methyl, ethyl, OH, cyano, CH₂—F, CHF₂ and CF₃, or a pharmaceutically acceptable salt thereof.

### EMBODIMENT 46

In certain variations of Embodiments 39 or 40, R⁴, R⁵, R⁶, and R⁷ are each hydrogen; and R⁸ selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, and unsubstituted or substituted NR^{c}R^{d}, wherein R^{c} and R^{d} are each independently selected from the group consisting of unsubstituted or substituted C₁₋₄alkyl, and unsubstituted or substituted C₃₋₆cycloalkyl; and wherein each R⁸ may be optionally substituted with one to two R^{e} groups independently selected from the group consisting of methyl, halogen, C₃₋₄cycloalkyl, and phenyl, or a pharmaceutically acceptable salt thereof.

### EMBODIMENT 47

In certain variations of Embodiments 44 or 45, R⁴, R⁵, R⁶, and R⁷ are each hydrogen; and R⁸ is selected from the group consisting of:

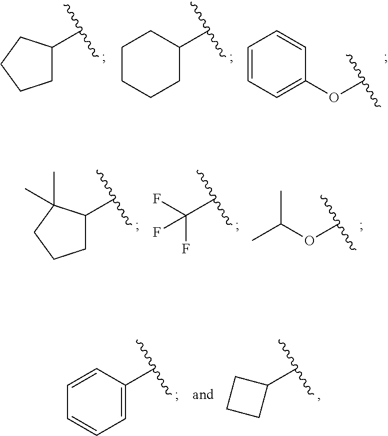

or a pharmaceutically acceptable salt thereof.

### EMBODIMENT 48

In certain variations of Embodiments 44 or 45, R¹⁰ and R¹¹ are each unsubstituted or substituted C₁₋₁₀alkyl; wherein each R¹⁰ and R¹¹ may be optionally substituted with one to five R^{e} groups, or a pharmaceutically acceptable salt thereof.

### EMBODIMENT 49

In certain variations of Embodiments 44 or 45, R¹⁰ and R¹¹ are cyclized to form an unsubstituted or substituted ring having 3-8 ring members; wherein the ring having 3-8 ring members may be optionally substituted with one to five R^{e} groups, or a pharmaceutically acceptable salt thereof.

### EMBODIMENT 50

In certain variations of Embodiments 44 or 45, each R¹⁰ and R¹¹ are optionally substituted with one to two R^{e} groups selected from the group consisting of halogen, OH, C₁₋₁₀alkyl, cyano, and C₃₋₈cycloalkyl.

In embodiments, the compounds of formula (I) include the compounds listed in Table 3 and stereoisomers thereof, tautomers thereof, and pharmaceutically acceptable salts thereof.

### III. Pharmaceutical Compositions and Administration

Another aspect includes a pharmaceutical composition comprising a compound of formula (I) or a pharmaceutically acceptable salt thereof. In one embodiment, the composition further comprises a pharmaceutically acceptable carrier, adjuvant, or vehicle. In another embodiment, the composition further comprises a therapeutically inert carrier. In another embodiment, the composition further comprises an amount of the compound effective to measurably disrupt the YAP:TEAD protein:protein interaction. In certain embodiments, the composition is formulated for administration to a patient in need thereof.

Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions comprising a compound of formula (I) or salt thereof may be administered orally, parenterally, by inhalation spray, topically, transdermally, rectally, nasally, buccally, sublingually, vaginally, intraperitoneal, intrapulmonary, intradermal, epidural or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

In one embodiment, the composition comprising a compound of formula (I) or salt thereof is formulated as a solid dosage form for oral administration. Solid dosage forms for oral administration include capsules, tablets, pills, powders, and granules. In certain embodiments, the solid oral dosage form comprising a compound of formula (I) or a salt thereof further comprises one or more of (i) an inert, pharmaceutically acceptable excipient or carrier, such as sodium citrate or dicalcium phosphate, and (ii) filler or extender such as starches, lactose, sucrose, glucose, mannitol, or silicic acid, (iii) binders such as carboxymethylcellulose, alginates, gelatin, polyvinylpyrrolidinone, sucrose or acacia, (iv) humectants such as glycerol, (v) disintegrating agent such as agar, calcium carbonate, potato or tapioca starch, alginic acid, certain silicates or sodium carbonate, (vi) solution retarding agents such as paraffin, (vii) absorption accelerators such as quaternary ammonium salts, (viii) a wetting agent such as cetyl alcohol or glycerol monostearate, (ix) absorbent such as kaolin or bentonite clay, and (x) lubricant such as talc, calcium stearate, magnesium stearate, polyethylene glycols or sodium lauryl sulfate. In certain embodiments, the solid oral dosage form is formulated as capsules, tablets or pills. In certain embodiments, the solid oral dosage form further comprises buffering agents. In certain embodiments, such compositions for solid oral dosage forms may be formulated as fillers in soft and hard-filled gelatin capsules comprising one or more excipients such as lactose or milk sugar, polyethylene glycols and the like.

In certain embodiments, tablets, dragees, capsules, pills and granules of the compositions comprising a compound of formula (I) or salt thereof optionally comprise coatings or shells such as enteric coatings. They may optionally comprise opacifying agents and can also be of a composition that they release the active ingredient(s) only, or preferentially, in a certain part of the intestinal tract, optionally, in a delayed manner. Examples of embedding compositions include polymeric substances and waxes, which may also be employed as fillers in soft and hard-filled gelatin capsules using such excipients as lactose or milk sugar as well as high molecular weight polethylene glycols and the like.

In another embodiment, a composition comprises micro-encapsulated compound of formula (I) or salt thereof, and optionally, further comprises one or more excipients.

In another embodiment, compositions comprise liquid dosage formulations comprising a compound of formula (I) or salt thereof for oral administration, and optionally further comprise one or more of pharmaceutically acceptable emulsions, microemulsions, solutions, suspensions, syrups and elixirs. In certain embodiments, the liquid dosage form optionally, further comprise one or more of an inert diluent such as water or other solvent, a solubilizing agent, and an emulsifier such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols or fatty acid esters of sorbitan, and mixtures thereof. In certain embodiments, liquid oral compositions optionally further comprise one or more adjuvant, such as a wetting agent, a suspending agent, a sweetening agent, a flavoring agent and a perfuming agent.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of formula (I), it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In certain embodiments, the composition for rectal or vaginal administration are formulated as suppositories which can be prepared by mixing a compound of formula (I) or a salt thereof with suitable non-irritating excipients or carriers such as cocoa butter, polyethylene glycol or a suppository wax, for example those which are solid at ambient temperature but liquid at body temperature and therefore melt in the rectum or vaginal cavity and release the compound of formula (I).

Example dosage forms for topical or transdermal administration of a compound of formula (I) include ointments, pastes, creams, lotions, gels, powders, solutions, sprays, inhalants or patches. The compound of formula (I) or a salt thereof is admixed under sterile conditions with a pharmaceutically acceptable carrier, and optionally preservatives or buffers. Additional formulation examples include an ophthalmic formulation, ear drops, eye drops, and transdermal patches. Transdermal dosage forms can be made by dissolving or dispensing the compound of formula (I) or a salt thereof in medium, for example ethanol or dimethylsulfoxide. Absorption enhancers can also be used to increase the flux of the compound across the skin. The rate can be controlled by either providing a rate controlling membrane or by dispersing the compound in a polymer matrix or gel.

Nasal aerosol or inhalation formulations of a compound of formula (I) or a salt thereof may be prepared as solutions in saline, employing benzyl alcohol or other suitable preservatives, absorption promotors to enhance bioavailability, fluorocarbons, and/or other conventional solubilizing or dispersing agents.

In certain embodiments, pharmaceutical compositions may be administered with or without food. In certain embodiments, pharmaceutically acceptable compositions are administered without food. In certain embodiments, pharmaceutically acceptable compositions of this invention are administered with food.

Specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided compound of formula (I) or salt thereof in the composition will also depend upon the particular compound in the composition.

In one embodiment, the therapeutically effective amount of the compound of the invention administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, contain from about 5 to about 100 mg of the compound of the invention.

An example tablet oral dosage form comprises about 2 mg, 5 mg, 25 mg, 50 mg, 100 mg, 250 mg or 500 mg of a compound of formula (I) or salt thereof, and further comprises about 5-30 mg anhydrous lactose, about 5-40 mg sodium croscarmellose, about 5-30 mg polyvinylpyrrolidone (PVP) K30 and about 1-10 mg magnesium stearate. The process of formulating the tablet comprises mixing the powdered ingredients together and further mixing with a solution of the PVP. The resulting composition can be dried, granulated, mixed with the magnesium stearate and compressed to tablet form using conventional equipment. An example of an aerosol formulation can be prepared by dissolving about 2-500 mg of a compound of formula (I) or salt thereof, in a suitable buffer solution, e.g. a phosphate buffer, and adding a tonicifier, e.g. a salt such sodium chloride, if desired. The solution may be filtered, e.g. using a 0.2 micron filter, to remove impurities and contaminants.

### IV. Indications and Combination Therapy

Compounds of the present disclosure are small molecule YAP:TEAD inhibitors. Small molecule YAP:TEAD inhibitors are useful, e.g., for the diagnosis or treatment of cancer, including with no limitations, lung cancer, breast cancer, head and neck cancer, colon cancer, ovarian cancer, liver cancer, brain cancer and prostate cancer, mesotheliomas, sarcomas and/or leukemia. In other embodiments, small molecule YAP:TEAD inhibitors are useful for the diagnosis or treatment of cancers characterized by solid tumors, including with no limitations lung, liver, ovarian, breast and/or squamous cancers. In some embodiments, the solid tumors have YAP/TAZ amplification or Nf2 deletion/mutation.

In some embodiments, the disclosure includes use of any of the compounds of formula (I) disclosed herein for the therapeutic and/or prophylactic treatment of cancer. In other embodiments, the disclosure includes use of any of the compounds of formula (I) disclosed herein for the preparation of a medicament for the therapeutic and/or prophylactic treatment of cancer. In other embodiments, the disclosure includes compounds of formula (I) disclosed herein for the therapeutic and/or prophylactic treatment of cancer.

In some embodiments, the disclosure includes methods for the therapeutic and/or prophylactic treatment of cancer, the method including administering an effective amount of a compound of formula (I) disclosed herein.

Breast Cancer

Compounds of the disclosure can be administered alone or they can be used in a combination therapy for the treatment of breast cancer. For instance, the combination therapy includes administering a compound of the disclosure and administering at least one additional therapeutic agent (e.g. one, two, three, four, five, or six additional therapeutic agents) for the treatment of breast cancer.

Standard of care for breast cancer is determined by both disease (tumor, stage, pace of disease, etc.) and patient characteristics (age, by biomarker expression and intrinsic phenotype). General guidance on treatment options are described in the NCCN Guidelines (e.g., NCCN Clinical Practice Guidelines in Oncology, Breast Cancer, version _{2.2016}, National Comprehensive Cancer Network, 2016, pp. 1-202), and in the ESMO Guidelines (e.g., Senkus, E., et al. Primary Breast Cancer: ESMO Clinical Practice Guidelines for diagnosis, treatment and follow-up. Annals of Oncology 2015; 26(Suppl. 5): v8-v30; and Cardoso F., et al. Locally recurrent or metastatic breast cancer: ESMO Clinical Practice Guidelines for diagnosis, treatment and follow-up. Annals of Oncology 2012; 23 (Suppl. 7):vii11-vii19.).

In some aspects, the compounds are for use in a combination therapy for the treatment of breast cancer in combination with one or more other therapeutic agents. In a further aspect, the compounds are for use in a combination therapy for the treatment of early breast cancer or locally advanced breast cancer. In a further aspect, the compounds are for use in a combination therapy for the treatment of advanced breast cancer or metastatic breast cancer.

In particular, compounds of the disclosure can be used either alone or in combination with standard of care treatment options for breast cancer, which in general include surgery, systemic chemotherapy (either pre- or post-operatively) and/or radiation therapy. Depending on tumor and patient characteristics, systemic chemotherapy may be administered as adjuvant (post-operative) therapy or as neoadjuvant (pre-operative) therapy.

Thus, in one embodiment, the combination therapy comprises administering a compound of the present disclosure and administering at least one additional therapeutic agent such as doxorubicin, epirubicin, cyclophosphamide, docetaxel, paclitaxel, methotrexate, and/or 5-fluorouracil.

In one embodiment, the combination therapy comprises administering a compound of the present disclosure and administering doxorubicin and cyclophosphamide (AC chemotherapy). In one embodiment, the combination therapy comprises administering a compound of the present disclosure and administering docetaxel, doxorubicin and cyclophosphamide (TAC chemotherapy). In one embodiment, the combination therapy comprises administering a compound of the present disclosure and administering cyclophosphamide, methotrexate and 5-fluorouracil (CMF chemotherapy). In one embodiment, the combination therapy comprises administering a compound of the present disclosure and administering epirubicin and cyclophosphamide (EC chemotherapy). In one embodiment, the combination therapy comprises administering a compound of the present disclosure and administering 5-fluorouracil, epirubicin and cyclophosphamide (FEC chemotherapy). In one embodiment, the combination therapy comprises administering a compound of the present disclosure and administering 5-fluorouracil, doxorubicin and cyclophosphamide (FAC chemotherapy). In one embodiment, the combination therapy comprises administering a compound of the present disclosure and administering taxane, in particular docetaxel or paclitaxel.

In one embodiment, when the compounds of the disclosure are for use in the treatment of metastatic breast cancer, the combination therapy comprises administering a compound of the present disclosure and administering at least one additional therapeutic agent such as doxorubicin, pegylated liposomal doxorubicin, epirubicin, cyclophosphamide, carboplatin, cisplatin, docetaxel, paclitaxel, albumin-bound paclitaxel, capecitabine, gemcitabine, vinorelbine, eribulin, Ixabepilone, methotrexate, and/or 5-fluorouracil (5-FU). In one embodiment, the combination therapy comprises administering a compound of the present disclosure and administering docetaxel and capecitabine for use in the treatment of metastatic breast cancer. In one embodiment, the combination therapy comprises administering a compound of the present disclosure and administering gemcitabine and paclitaxel for use in the treatment of metastatic breast cancer.

Breast Cancer Hormone Receptor Positive (ER+ and/or PR+)

In a further aspect, the disclosure provides a method for treating hormone receptor positive (HR+) breast cancer (also called estrogen receptor positive (ER+) breast cancer or estrogen receptor positive and/or progesterone receptor positive (PR+) breast cancer), by administering an effective amount of a compound of the present disclosure. In a further aspect of the embodiment, the breast cancer is early or locally advanced hormone receptor positive (HR+) breast cancer, also named early or locally advanced ER+ breast cancer. In a further aspect, the breast cancer is advanced hormone receptor positive (HR+) breast cancer or metastatic hormone receptor positive (HR+) breast cancer, also named advanced ER+ breast cancer or metastatic ER+ breast cancer.

In some aspects, the compounds are for use in a combination therapy for the treatment of hormone receptor positive (HR+) breast cancer or estrogen receptor positive (ER+) breast cancer. In a further aspect, the compounds are for use in a combination therapy for the treatment of early or locally advanced hormone receptor positive (HR+) breast cancer, also named early or locally advanced ER+ breast cancer. In a further aspect of the embodiment, the compounds are for use in a combination therapy for the treatment of advanced hormone receptor positive (HR+) breast cancer or metastatic hormone receptor positive (HR+) breast cancer, also named advanced ER+ breast cancer or metastatic ER+ breast cancer. In one embodiment, the method comprises administering to an individual having hormone receptor positive (HR+) breast cancer or estrogen receptor positive (ER+) breast cancer an effective amount of a compound of the present disclosure in combination with one or more other therapeutic agents.

In particular, compounds of the disclosure can be used either alone or in combination with standard of care treatment options for hormone receptor positive (HR+) breast cancer or estrogen receptor positive (ER+) breast cancer, which in general include surgery, systemic chemotherapy (either pre- or post-operatively) and/or radiation therapy. Depending on tumor and patient characteristics, systemic chemotherapy may be administered as adjuvant (post-operative) therapy or as neoadjuvant (pre-operative) therapy.

In one embodiment, compounds of the disclosure are for use in the treatment of hormone receptor positive (HR+) breast cancer or estrogen receptor positive (ER+) breast cancer in combination with endocrine therapy. In one embodiment, the combination therapy comprises administering a compound of the present disclosure and administering tamoxifen. In one embodiment, the combination therapy comprises administering an a compound of the present disclosure and administering an aromatase inhibitor, such as anastrozole, letrozole or exemestane for use in the treatment of hormone receptor positive (HR+) breast cancer or estrogen receptor positive (ER+) breast cancer. In one embodiment, the combination therapy comprises administering an a compound of the present disclosure and administering at least one additional therapeutic agent such as anastrozole, letrozole, exemestane and everolimus, palbociclib and letrozole, palbociclib and letrozole, fulvestrant, tamoxifen, toremifene, megestrol acetate, fluoxemesterone, and/or ethinyl estradiol for use in the treatment of hormone receptor positive (HR+) breast cancer or estrogen receptor positive (ER+) breast cancer.

In one embodiment, compounds of the disclosure are for use in the treatment of hormone receptor positive (HR+) breast cancer or estrogen receptor positive (ER+) breast cancer in combination with one or more chemotherapeutic agents. In one embodiment, the combination therapy comprises administering a compound of the present disclosure and administering at least one additional therapeutic agent such as doxorubicin, epirubicin, cyclophosphamide, docetaxel, paclitaxel, methotrexate, and/or 5-fluorouracil for use in the treatment of hormone receptor positive (HR+) breast cancer or estrogen receptor positive (ER+) breast cancer.

In one aspect, compounds of the disclosure are for use in combination with doxorubicin and cyclophosphamide (AC chemotherapy). In one embodiment, the combination therapy comprises administering a compound of the present disclosure and administering docetaxel, doxorubicin and cyclophosphamide (TAC chemotherapy). In one embodiment, the combination therapy comprises administering a compound of the present disclosure and administering cyclophosphamide, methotrexate and 5-fluorouracil (CMF chemotherapy). In one embodiment, the combination therapy comprises administering a compound of the present disclosure and administering epirubicin and cyclophosphamide (EC chemotherapy). In one embodiment, the combination therapy comprises administering a compound of the present disclosure and administering 5-fluorouracil, epirubicin and cyclophosphamide (FEC chemotherapy). In one embodiment, the combination therapy comprises administering a compound of the present disclosure and administering 5-fluorouracil, doxorubicin and cyclophosphamide (FAC chemotherapy). In one embodiment, the combination therapy comprises administering a compound of the present disclosure and administering a taxane, such as docetaxel or paclitaxel.

In one embodiment, compounds of the disclosure are for use in the treatment of metastatic breast cancer. In one embodiment, the combination therapy comprises administering an a compound of the present disclosure and administering doxorubicin, pegylated liposomal doxorubicin, epirubicin, cyclophosphamide, carboplatin, cisplatin, docetaxel, paclitaxel, albumin-bound paclitaxel, capecitabine, gemcitabine, vinorelbine, eribulin, ixabepilone, methotrexate and 5-fluorouracil (5-FU) for use in the treatment of metastatic breast cancer. In one embodiment, the combination therapy comprises administering a compound of the present disclosure and administering docetaxel and capecitabine for use in the treatment of metastatic breast cancer. In one embodiment, the combination therapy comprises administering a compound of the present disclosure and administering gemcitabine and paclitaxel for use in the treatment of metastatic breast cancer.

Breast Cancer—HER2+

In a further aspect, the disclosure provides a method for treating Her2+ positive breast cancer, by administering an effective amount of a compound of the present disclosure. In a further aspect of the embodiment, the breast cancer is early or locally advanced Her2+ positive breast cancer, also named early or locally advanced Her2+ positive breast cancer. In a further aspect, the breast cancer is advanced breast cancer, also named advanced Her2+ positive breast cancer or metastatic ER+ breast cancer.

In some aspects, the compounds are for use in a combination therapy for treatment of Her2+ positive breast cancer. In a further aspect, the compounds are for use in a combination therapy for treatment of early or locally advanced Her2+ positive breast cancer, also named early or locally advanced Her2+ positive breast cancer. In a further aspect of the embodiment, the compounds are for use in a combination therapy for treatment of advanced Her2+ positive breast cancer, also named advanced Her2+ positive breast cancer or metastatic ER+ breast cancer. In one embodiment, the method comprises administering to an individual having Her2+ positive breast cancer an effective amount of a compound of the present disclosure in combination with one or more other therapeutic agents.

In particular, compounds of the disclosure can be used either alone or in combination with standard of care treatment options for Her2+ positive breast cancer, which in general include surgery, systemic chemotherapy (either pre- or post-operatively) and/or radiation therapy. Depending on tumor and patient characteristics, systemic chemotherapy may be administered as adjuvant (post-operative) therapy or as neoadjuvant (pre-operative) therapy.

In one embodiment, the combination therapy comprises administering a compound of the present disclosure and administering a Her2 antibody to treat Her2+ positive breast cancer. In one aspect, the combination therapy comprises administering a compound of the present disclosure and administering trastuzumab or pertuzumab to treat Her2+ positive breast cancer. In another aspect, the combination therapy comprises administering a compound of the present disclosure and administering a chemotherapy to treat Her2+ positive breast cancer. In one embodiment, the combination therapy comprises administering a compound of the present disclosure and administering doxorubicin and cyclophosphamide followed by trastuzumab to treat Her2+ positive breast cancer. In a further embodiment, compounds of the disclosure are for use in the treatment of Her2+ positive breast cancer in combination with chemotherapy followed by a taxane and trastuzumab to treat Her2+ positive breast cancer. In another aspect, compounds of the disclosure are for use in the treatment of Her2+ positive breast cancer in combination with trastuzumab (Herceptin) and pertuzumab (Perjeta) to treat Her2+ positive breast cancer.

In another aspect, compounds of the disclosure are used in combination with docetaxel, carboplatin and trastuzumab (TCH chemotherapy). In a further aspect, compounds of the disclosure are administered in combination with docetaxel, carboplatin, trastuzumab and pertuzumab. In a further aspect, compounds of the disclosure are administered in combination with 5-fluorouracil, epirubicin and cyclophosphamide (FEC chemotherapy and pertuzumab, trastuzumab and docetaxel or paclitaxel. In another aspect, compounds of the disclosure are used in combination with paclitaxel and trastuzumab. In a further aspect, compounds of the disclosure are administered in combination with Pertuzumab and trastuzumab and paclitaxel or docetaxel.

If the compounds of the disclosure are for use in the treatment of metastatic Her2+ positive breast cancer, they can also be used in combination with one or more chemotherapeutic agents selected from the group consisting of doxorubicin (A) (Adriamycin),pegylated liposomal doxorubicin (Doxil), epirubicin (E) (Ellence), cyclophosphamide (C) (Cytoxan), carboplatin (Platinol), cisplatin (Paraplatin), docetaxel (T) (Taxotere), paclitaxel (Taxol), albumin-bound paclitaxel (Abraxane), capecitabine (Xeloda), gemcitabine (Cynzar), vinorelbine (Navelbine), eribulin (Halaven), and Ixabepilone (Ixempra), In one aspect, the compounds of the disclosure are for use in the treatment of metastatic Her2+ positive breast cancer in combination with ado-trastuzumab emtansine (T-DM1).

In a particular aspect, compounds of the disclosure are for use in the treatment of metastatic Her2+ positive breast cancer in combination with trastuzumab and pertuzumab and a taxane. In one aspect, the taxane is docetaxel. In another aspect, the taxane is paclitaxel.

Breast Cancer—Triple Negative

Compounds of the disclosure can be used either alone or in a combination therapy with standard of care treatment options for triple negative breast cancer (TNBC), which in general include surgery, systemic chemotherapy (either pre- or post-operatively) and/or radiation therapy.

Standard of care for TNBC is determined by both disease (stage, pace of disease, etc.) and patient (age, co-morbidities, symptoms, etc.) characteristics. General guidance on treatment options are described in the NCCN Guidelines (e.g., NCCN Clinical Practice Guidelines in Oncology, Breast Cancer, version 2.2016, National Comprehensive Cancer Network, 2016, pp. 1-202), and in the ESMO Guidelines (e.g., Senkus, E., et al. Primary Breast Cancer: ESMO Clinical Practice Guidelines for diagnosis, treatment and follow-up. Annals of Oncology 2015; 26(Suppl. 5): v8-v30; and Cardoso F., et al. Locally recurrent or metastatic breast cancer: ESMO Clinical Practice Guidelines for diagnosis, treatment and follow-up. Annals of Oncology 2012; 23 (Suppl. 7):vii11-vii19.). See also, Rodler, E, et al. Breast Disease. 2010/2011; 32:99-122.

Metastatic TNBC

Systemic chemotherapy is the standard treatment for patients with metastatic TNBC, although no standard regimen or sequence exists. Single-agent cytotoxic chemotherapeutic agents as shown in Table 1 are generally regarded as the primary option for patients with metastatic TNBC, although combination chemotherapy regimens such as those shown in Table 2 may be used, for instance when there is aggressive disease and visceral involvement. Additional details on chemotherapy combinations that can be utilized are provided below in the section on early and locally advanced treatment options. Treatment may also involve sequential rounds of different single agent treatments. Palliative surgery and radiation may be utilized as appropriate to manage local complications.

The methods provided herein include administering a compound of the present disclosure to a patient with metastatic TNBC in combination with one of the single-agent chemotherapy agents listed in Table 1 or in combination with sequential rounds of different chemotherapy agents listed in Table 1. Such methods may optionally be combined with surgery and/or radiation treatment.

TABLE 1
Single agent chemotherapy regimens

| Class | Typical agents |
|---|---|
| Anthracyclines | Doxorubicin |
| | Pegylated liposomal doxorubicin |
| | Epirubicin |
| Taxanes | Paclitaxel |
| | Docetaxel |
| | Albumin-bound paclitaxel (nab-paclitaxel) |
| Anti-metabolites | Capecitabine |
| | Gemcitabine |
| Non-taxane | Vinorelbine |
| microtubule | Eribulin |
| inhibitors | Ixabepilone |
| Platinum | Carboplatin |
| | Cisplatin |
| Alkylating agent | Cyclophosphamide |

In one embodiment, the combination therapy comprises administering a compound of the present disclosure and administering an anthracycline such as doxorubicin, pegylated liposomal doxorubicin, or epirubicin.

In one embodiment, the combination therapy comprises administering a compound of the present disclosure and administering a taxane such as paclitaxel, docetaxel or albumin-bound paclitaxel (e.g., nab-paclitaxel).

In one embodiment, the combination therapy comprises administering a compound of the present disclosure and administering an anti-metabolite, including, for example, capecitabine or gemcitabine.

In one embodiment, the combination therapy comprises administering a compound of the present disclosure and administering a non-taxane microtubule inhibitor, such as vinorelbine, eribulin or ixabepilone.

In one embodiment, the combination therapy comprises administering a compound of the present disclosure and administering a platinum compound, such as carboplatin or cisplatin.

In one embodiment, the combination therapy comprises administering a compound of the present disclosure and administering an alkylating agent such as cyclophosphamide.

In some embodiments, a compound of the present disclosure is administered with a combination of chemotherapy agents as summarized in Table 2 below.

Additional guidance for treating metastatic TNBC is provided in Jones S E, et al. J Clin Concol. 2006; 24:5381-5387; Heemskerk-Gerritsen BAM, et al. Ann Surg. Oncol. 2007; 14:3335-3344; and Kell M R, et al. MBJ. 2007; 334:437-438.

Early and Locally Advanced TNBC

Patients with early and potentially resectable locally advanced TNBC (i.e. without distant metastatic disease) are managed with locoregional therapy (surgical resection with or without radiation therapy) with or without systemic chemotherapy.

Surgical treatment can be breast-conserving (e.g., a lumpectomy, which focuses on removing the primary tumor with a margin), or can be more extensive (e.g., mastectomy, which aims for complete removal of all of the breast tissue). Radiation therapy is typically administered post-surgery to the breast/chest wall and/or regional lymph nodes, with the goal of killing microscopic cancer cells left post-surgery. In the case of a breast conserving surgery, radiation is administered to the remaining breast tissue and sometimes to the regional lymph nodes (including axillary lymph nodes). In the case of a mastectomy, radiation may still be administered if factors that predict higher risk of local recurrence are present.

In one embodiment, a compound of the present disclosure is administered in combination with surgical treatment, either as a neoadjuvant or adjuvant therapy. In another embodiment, a compound of the present disclosure is administered before or after radiation treatment. In still another embodiment, a compound of the present disclosure is administered in combination with surgical and radiation treatment.

Depending on tumor and patient characteristics, chemotherapy may be administered in the adjuvant (post-operative) or neoadjuvant (pre-operative) setting. Examples of adjuvant/neoadjuvant chemotherapy regimens used to treat TNBC recommended by current guidelines are shown in Table 2. A compound of the present disclosure can be combined with any of the regimens shown in Table 2.

TABLE 2
Combination chemotherapy regimens

| Class | Typical agents | Shorthand |
|---|---|---|
| Anthracycline and | Doxorubicin + cyclophosphamide | AC→ T |
| alkylating agent | followed by a taxane (e.g., docetaxel | |
| followed by | or paclitaxel) | |
| taxane | | |
| Anthracycline and | Doxorubicin + cyclophosphamide | AC |
| alkylating agent | (or liposomal doxorubicin + | EC |
| | cyclophosphamide) Epirubicin + | |
| | cyclophosphamide | |
| Taxane, | Docetaxel + doxorubicin + | TAC |
| anthracycline, and | cyclophosphamide | |
| alkylating agent | | |
| Taxane and | Docetaxel + cyclophosphamide | TC |
| alkylating agent | | |
| Alkylating agent, | Cyclophosphamide + methotrexate + | CMF |
| methotrexate and | fluorouracil | |
| anti-metabolite | | |
| Anti-metabolite, | Fluorouracil + doxorubicin + | FAC |
| anthracycline, and | cyclophosphamide | FEC |
| alkylating agent | Fluorouracil + epirubicin + | |
| | cyclophosphamide | |
| Anti-metabolite, | Fluorouracil + epirubicin + | FEC/ |
| anthracycline, and | cyclophosphamide followed by docetaxel | CEF→T |
| alkylating agent | or paclitaxel | FAC→T |
| followed by | Fluorouracil + doxorubicin + | |
| taxane | cyclophosphamide followed by paclitaxel | |
| Taxane and anti- | Docetaxel + capecitabine, or | GT |
| metabolite | Paclitaxel + gemcitabine | |
| Anti-metabolite | Gemcitabine + carboplatin | |
| and platinum | | |
| Anti-metabolite | Capecitibine + vinorelbine | |
| and non-taxane | Gemcitabine + vinorelbine | |
| microtubule | | |
| inhibitor | | |
| Taxane and | Paclitaxel + bevacizumab | |
| VEGFinhibitor | | |
| (e.g., anti-VEGF | | |
| antibody) | | |

In one embodiment, the combination therapy comprises administering a compound of the present disclosure and administering an anthracycline and an alkylating agent, optionally followed by a taxane. In one such embodiment, the compound of the present disclosure is administered with doxorubicin and cyclophosphamide followed by a taxane (e.g., docetaxel or paclitaxel), which is a chemotherapy regimen designated as AC→T.

In one embodiment, the combination therapy comprises administering a compound of the present disclosure and administering an anthracycline and an alkylating agent. For example, in one embodiment, the combination therapy comprises administering a compound of the present disclosure and administering doxorubicin or liposomal doxorubicin and cyclophosphamide, which is designated as AC. In another embodiment, the combination therapy comprises administering a compound of the present disclosure and administering epirubicin and cyclophosphamide, which is a chemotherapy regimen referred to as EC.

In one embodiment, the combination therapy comprises administering a compound of the present disclosure and administering a taxane, an anthracycline, and an alkylating agent. For instance, in one embodiment, the combination therapy comprises administering a compound of the present disclosure and administering docetaxel, doxorubicin and cyclophosphamide, a chemotherapy regimen which is denoted as TAC.

In another embodiment, the combination therapy comprises administering a compound of the present disclosure and administering taxane and an alkylating agent. In one such embodiment, the combination therapy comprises administering a compound of the present disclosure and administering docetaxel and cyclophosphamide, which is a chemotherapy regimen referred to as TC.

In still another embodiment, the combination therapy comprises administering a compound of the present disclosure and administering taxane and an alkylating agent. For instance, in one embodiment, the combination therapy comprises administering a compound of the present disclosure and administering docetaxel and cyclophosphamide, a chemotherapy regimen designated as TC.

In one embodiment, the combination therapy comprises administering a compound of the present disclosure and administering an alkylating agent, methotrexate, and an anti-metabolite. As an example, in one embodiment, the combination therapy comprises administering a compound of the present disclosure and administering an alkylating agent, methotrexate and an anti-metabolite. In one such embodiment, the combination therapy comprises administering a compound of the present disclosure and administering cyclophosphamide, methotrexate and fluorouracil, a chemotherapy regimen which is referred to as CMF.

In another embodiment, the combination therapy comprises administering a compound of the present disclosure and administering an anti-metabolite, an anthracycline, and an alkylating agent. In one such embodiment, the combination therapy comprises administering a compound of the present disclosure and administering fluorouracil, doxorubicin and cyclophosphamide, which is a chemotherapy regimen denoted as FAC. In another such embodiment, the combination therapy comprises administering a compound of the present disclosure and administering fluorouracil, epirubicin and cyclophosphamide, a chemotherapy regimen designated as FEC.

In still another embodiment, the combination therapy comprises administering a compound of the present disclosure and administering an anti-metabolite, an anthracycline, and an alkylating agent followed by taxane. As an example, in one embodiment, the combination therapy comprises administering a compound of the present disclosure and administering fluorouracil, epirubicin and cyclophosphamide followed by docetaxel or paclitaxel, a chemotherapy regimen referred to as FEC (or CEF)→T. In another embodiment, the combination therapy comprises administering a compound of the present disclosure and administering fluorouracil, doxorubicin and cyclophosphamide followed by paclitaxel, which is a chemotherapy regimen designated as FAC T.

In yet another embodiment, the combination therapy comprises administering a compound of the present disclosure and administering taxane and an anti-metabolite. As an example, in one embodiment, the combination therapy comprises administering a compound of the present disclosure and administering docetaxel and capecitabine. In another example the combination therapy comprises administering a compound of the present disclosure and administering paclitaxel and gemcitabine, a chemotherapy regimen referred to as GT.

In one embodiment, the combination therapy comprises administering a compound of the present disclosure and administering an anti-metabolite and a platinum compound. For instance, in one embodiment, the combination therapy comprises administering a compound of the present disclosure and administering gemcitabine and carboplatin.

In another embodiment, the combination therapy comprises administering a compound of the present disclosure and administering an anti-metabolite and a non-taxane microtubule inhibitor. In one such embodiment, the combination therapy comprises administering a compound of the present disclosure and administering capecitibine and vinorelbine. In another such embodiment, the combination therapy comprises administering a compound of the present disclosure and administering gemcitabine and vinorelbine.

In still another embodiment, the combination therapy comprises administering a compound of the present disclosure and administering a taxane and a VEGF inhibitor (e.g., anti-VEGF antibody). For instance, in one embodiment, the combination therapy comprises administering a compound of the present disclosure and administering paclitaxel and bevacizumab.

Additional guidance for treating early and locally advanced TNBC is provided in Solin L J., Clin Br Cancer. 2009; 9:96-100; Freedman G M, et al. Cancer. 2009; 115:946-951; Heemskerk-Gerritsen BAM, et al. Ann Surg Oncol. 2007; 14:3335-3344; and Kell M R, et al. MBJ. 2007; 334:437-438.

Non-small cell Lung Cancer (NSCLC)

Compounds of the disclosure can be administered alone or they can be used in a combination therapy. For instance, the combination therapy includes administering a compound of the disclosure and administering at least one additional therapeutic agent (e.g. one, two, three, four, five, or six additional therapeutic agents).

In some aspects, the compounds are for use in a combination therapy for the treatment of nonsmall cell lung cancer NSCLC, such as a squamous cell carcinoma, adenocarcinoma, large cell carcinoma, adenosquamous carcinoma, undifferentiated carcinoma, or a combination thereof.

In one embodiment, the NSCLC is in occult stage, stage 0, I, II, III, or IV.

In one embodiment, the NSLCL is in occult stage, stage 0, IA, IB, IIA, IIB, IIIA, HIB, or IV.

The present disclosure is directed to use of disclosed compounds for an adjuvant or neo-adjuvant treatment.

The present disclosure is directed to use of disclosed compounds for a first line, second line, or third line treatment.

The present disclosure is directed to use of disclosed compounds for a single agent treatment.

The present disclosure is directed to use of disclosed compounds for a treatment of a stage IV or a recurrent disease.

The present disclosure is directed to use of disclosed compounds for a treatment which is combined with surgery, radiation therapy, or a combination thereof.

In one embodiment, the combination therapy comprises administering a compound of the present disclosure and administering at least one additional therapeutic agent such as cisplatin, carboplatin, paclitaxel, paclitaxel protein bound, docetaxel, gemcitabine, vinorelbine, etoposide, nintedanib, vinblastine, and/or pemetrexed.

In one embodiment, the combination therapy comprises administering a compound of the present disclosure and administering at least one additional therapeutic agent such as afatinib, bevacizumab, cabozantinib, ceritinib, crizotinib, erlotinib hydrochloride, osimertinib, ramucirumab, gefitinib, alectinib, trastuzumab, cetuximab, ipilimumab, trametinib, dabrafenib, vemurafenib, dacomitinib, tivantinib, and/or onartuzumab.

In one embodiment, the combination therapy comprises administering a compound of the present disclosure and administering at least one additional therapeutic agent such as afatinib, crizotinib, erlotinib hydrochloride, and/or gefitinib.

In one embodiment, the combination therapy comprises administering a compound of the present disclosure and administering checkpoint inhibitor agents, such as pembrolizumab, atezolizumab, and/or nivolumab.

In one embodiment, the combination therapy comprises administering a compound of the present disclosure and administering at least one additional therapeutic agent such as cisplatin, carboplatin, paclitaxel, paclitaxel protein bound, docetaxel, gemcitabine, vinorelbine, etoposide, nintedanib, vinblastine, pemetrexed, afatinib, bevacizumab, cabozantinib, ceritinib, crizotinib, erlotinib hydrochloride, osimertinib, ramucirumab, gefitinib, necitumumab, alectinib, trastuzumab, cetuximab, ipilimumab, trametinib, dabrafenib, vemurafenib, dacomitinib, tivantinib, onartuzumab, pembrolizumab, atezolizumab, and/or nivolumab.

Small cell lung Cancer (SCLC)

Compounds of the disclosure can be administered alone or they can be used in a combination therapy. For instance, the combination therapy includes administering a compound of the disclosure and administering at least one additional therapeutic agent (e.g. one, two, three, four, five, or six additional therapeutic agents).

In some aspects, the compounds are for use in a combination therapy for the treatment of Small Cell Lung Cancer (SCLC).

In one embodiment, the SCLC is a small cell carcinoma (oat cell cancer), mixed small cell/large cell carcinoma or combined small cell carcinoma.

In one embodiment, the SCLC is in occult stage, stage 0, I, II, III, or IV.

In one embodiment, the SLCL is in occult stage, stage 0, IA, IB, HA, IIB, IIIA, IIIB, or

IV.

In one embodiment, the SLCL is in stage I-III (limited stage).

The present disclosure is directed to use of disclosed compounds for a first line treatment of stage IV (extensive stage).

The present disclosure is directed to use of disclosed compounds for a second line treatment of stage IV (relapsed or refractory disease).

The present disclosure is directed to use of disclosed compounds for a third line treatment of stage IV (relapsed or refractory disease).

In one embodiment, a compound of the present disclosure is administered with one or more additional therapeutic agents selected from Etoposide, a platinum compound, Irinotecan, Topotecan, vinca alkaloids, alkylating agents, Doxorubicin, taxanes, and Gemcitabine. In another embodiment, the platinum compound is Cisplatin or Carboplatin. In another embodiment, the vinca alkaloid is Vinblastine, Vincristine, or Vinorelbine. In another embodiment, the alkylating agent is Cyclophosphamide or Ifosfamide. In another embodiment, the taxane is Docetaxel or Paclitaxel.

Ovarian Cancer

In a further aspect, the disclosure provides a method for treating an ovarian cancer (such as epithelial ovarian cancer (EOC), ovarian germ cell tumors, or ovarian stromal tumors) by administering an effective amount of a compound of the present disclosure. In a further aspect of the embodiment, the ovarian cancer is an epithelial ovarian cancer (EOC). In a further aspect of the embodiment, the ovarian cancer is an ovarian germ cell tumor. In a further aspect of the embodiment, the ovarian cancer is an ovarian stromal cell tumor. In one embodiment, the method comprises administering to an individual having ovarian cancer an effective amount of a compound of the present disclosure.

Compounds of the disclosure can be administered alone or they can be used in a combination therapy to treat ovarian cancer. For instance, the combination therapy includes administering a compound of the disclosure and administering at least one additional therapeutic agent (e.g. one, two, three, four, five, or six additional therapeutic agents).

In some aspects, the compounds are for use in a combination therapy for the treatment of an ovarian cancer (such as epithelial ovarian cancer (EOC), ovarian germ cell tumors, or ovarian stromal tumors). In one embodiment, the combination therapy comprises administering a compound of the present disclosure and administering at least one additional therapeutic agent such as a platinum compound (such as carboplatin, cisplatin, less often oxaliplatin or iproplatin), and/or a taxane (such as paclitaxel or docetaxel, or albumin bound paclitaxel (nab-paclitaxel)). In one embodiment, the combination therapy comprises administering a compound of the present disclosure and administering carboplatin and a taxane (such as paclitaxel or docetaxel or Albumin bound paclitaxel (nab-paclitaxel)).

In one embodiment, the combination therapy comprises administering a compound of the present disclosure and administering at least one additional therapeutic agent such as albumin bound paclitaxel (nab-paclitaxel), altretamine, capecitabine, cyclophosphamide, etoposide, gemcitabine, ifosfamide, irinotecan, liposomal doxorubicin, melphalan, pemetrexed, topotecan, vinorelbine, bevacizumab, a platinum compound (such as carboplatin, cisplatin, oxaliplatin, or iproplatin), and/or a taxane (such as paclitaxel or docetaxel, or albumin bound paclitaxel (nab-paclitaxel)).

In one embodiment, the combination therapy comprises administering a compound of the present disclosure and administering bevacizumab and a taxane (such as paclitaxel or docetaxel, or albumin bound paclitaxel (nab-paclitaxel)).

In one embodiment, the combination therapy comprises administering a compound of the present disclosure and administering at least one additional therapeutic agent such as cisplatin, etoposide, and/or bleomycin.

In one embodiment, the combination therapy comprises administering a compound of the present disclosure and administering cisplatin (Platinol), etoposide, and bleomycin (PEB (or BEP)).

In one embodiment, the combination therapy comprises administering a compound of the present disclosure and administering paclitaxel (Taxol), ifosfamide, and cisplatin (TIP).

In one embodiment, the combination therapy comprises administering a compound of the present disclosure and administering vinblastine, ifosfamide, and cisplatin (VeIP).

In one embodiment, the combination therapy comprises administering a compound of the present disclosure and administering etoposide (VP-16), ifosfamide, and cisplatin (VIP).

### V. Methods of Manufacturing

In another embodiment, processes for making the subject compound are provided.

Referring to Scheme I, there is shown a general synthetic procedure for making compounds of the disclosure.

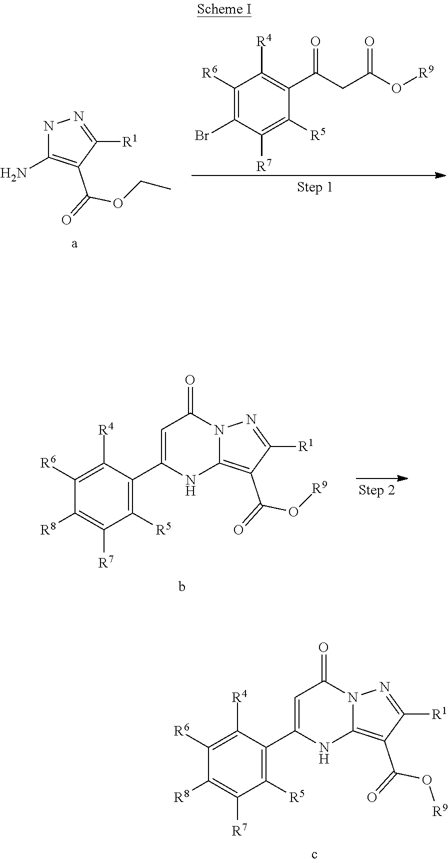

In step 1 of Scheme I, an aryl halide compound is reacted with compound IIa to yield compound IIb. The reaction may be carried out in a solvent such as acetic acid at a temperature of about 120-150° C. for about 10-20 hours. In some embodiments, the temperature is about 130° C. and the reaction time is about 16 hours.

In step 2 of Scheme I, a Pd catalyzed coupling reaction is carried out by reacting compound IIb with the appropriate R⁸-borolane reagent and a Pd catalyst to produce compound IIc. In some embodiments, the Pd catalyst is Pd(dppf)Cl₂. The reaction takes place in the presence of a base, such as Na₂CO₃, in a solvent such as water and 1,4-dioxane at a temperature of about 80-120° C. for about 10-20 hours. In some embodiments, the temperature is about 100° C. and the reaction time is about 16 hours.

The disclosure includes compounds when manufactured according to the above process outlined in Scheme I. Exemplary structures and compounds that may be manufactured by the process outlined in Scheme I are included in Embodiments 3-11.

In another embodiment, processes for making the subject compound are provided as shown in Scheme II.

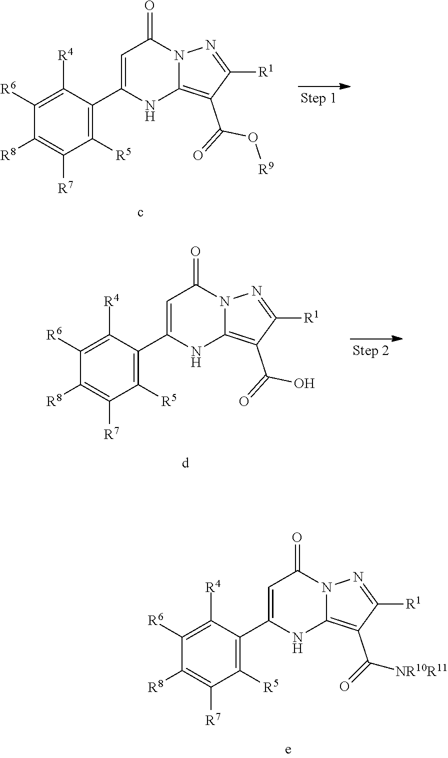

In step 1 of Scheme II, compound IIc is hydrolyzed to compound IId. In some embodiments, the hydrolysis takes place in the presence of a base in a solvent such as ethanol and water at a temperature of about 50-100° C. and a reaction time of about 10-20 hours. In some particular embodiments, the base is lithium hydroxide monohydrate, the temperature of the reaction is 75° C. and the reaction time is about 16 hours.

In step 2 of Scheme II, compound IId is reacted with the appropriate amine, NHR¹⁰R¹¹ in the presence of a base in a solvent such as DMF at a temperature of about 10-30° C. and a reaction time of about 10-20 hours to produce compound IIe. In some embodiments, the temperature of the reaction is 20° C. and the reaction time is 16 hours.

The disclosure includes compounds that are manufactured according to the above process outlined in Scheme II. Exemplary structures and compounds that may be manufactured by the process outlined in Scheme II are included in Embodiments 12-19.

### VI. Examples

The following are examples of methods and compositions of the disclosure. It is understood that various other embodiments may be practiced, given the general description provided above. The disclosure will be more fully understood by reference to the following examples. The claims should not, however, be construed as limited to the scope of the examples.

Intermediates and final compounds were purified by either flash chromatography, and/or by reverse-phase preparative HPLC (high performance liquid chromatography), and/or by supercritical fluid chromatography (SFC). Unless otherwise noted, flash chromatography was carried out using pre-packed silica gel cartridges from either ISCO or SiliCycle on an ISCO CombiFlashR chromatography instrument (from Teledyne Isco, Inc.).

Liquid chromatography-mass spectrometry (LCMS) was performed using a (1) Agilent technologies 6110/6120/G1946/G1925B Quadrupole in ESI+ mode, or (2) Shimadzu liquid chromatography-mass spectrometry (LCMS) 2010 mass spectrometer in ESI+ mode. Mass spectra data generally only indicates the parent ions unless otherwise stated. (MS or HRMS data is provided for a particular intermediate or compound where indicated.)

Nuclear magnetic resonance spectroscopy (NMR) was performed using a (1) Bruker 400 NMR spectrometer, or (2) Varian 400 NMR spectrometer, and referenced to tetramethylsilane. NMR data is provided for a particular intermediate or compound where indicated.

### EXAMPLE 1

### Ethyl 5-(4-(4,4-difluorocyclohexyl)phenyl)-7-oxo-4,7-dihydropyrazolo[1,5-a]pyrimidine-3-carboxylate

The reaction scheme was as follows:

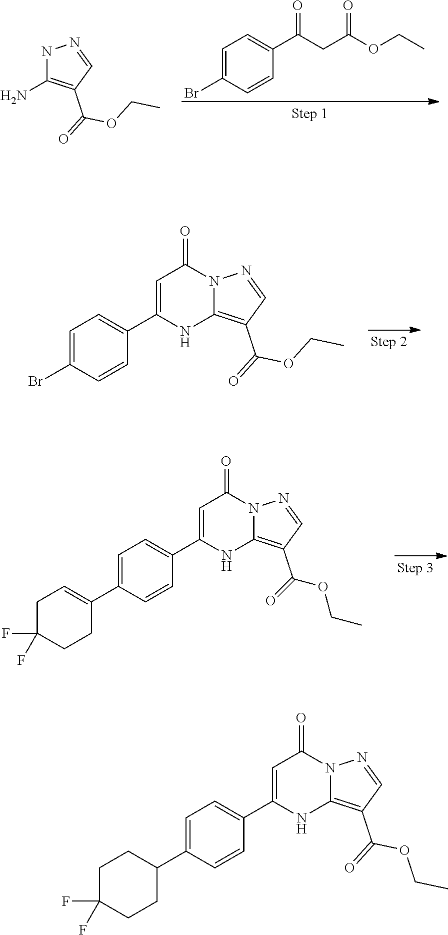

Step 1: Ethyl 5-(4-bromophenyl)-7-oxo-4,7-dihydropyrazolo[1,5-a]pyrimidine-3-carboxylate

A mixture of ethyl 3-(4-bromophenyl)-3-oxo-propanoate (2 g, 7.38 mmol) and ethyl 5-amino-1H-pyrazole-4-carboxylate (1 g, 6.64 mmol) in AcOH (10 mL) was stirred at 130° C. for 16 h. The reaction solution was poured into H₂O (100 mL), adjusted to pH 8 with saturated aq. NaHCO₃ solution, and extracted with DCM (50 mL×3). The combined organic layers were dried over Na₂SO₄, filtered, and concentrated under reduced pressure. The resultant crude product was triturated with MeOH (20 mL) to give ethyl 5-(4-bromophenyl)-7-oxo-4,7-dihydropyrazolo[1,5-a]pyrimidine-3-carboxylate as a white solid (0.8 g, 29% yield). ¹H NMR (400 MHz, DMSO-d₆) 11.85 (s, 1H), 8.23 (s, 1H), 7.77 (s, 4H), 6.25 (s, 1H), 4.29 (q, J=6.8 Hz, 2H), 1.33 (q, J=6.8 Hz, 3H); LCMS (ESI+) m/z 362 (M+H)⁺.

Step 2: Ethyl 5-(4′,4′-difluoro-2′,3′,4′,5′-tetrahydro-[1,1′-biphenyl]-4-yl)-7-oxo-4,7-dihydropyrazolo[1,5-a]pyrimidine-3-carboxylate

A mixture of ethyl 5-(4-bromophenyl)-7-oxo-4,7-dihydropyrazolo[1,5-a]pyrimidine-3-carboxylate (200 mg, 0.55 mmol), Pd(dppf)Cl₂ (40 mg, 0.06 mmol), Na₂CO₃ (175 mg, 1.66 mmol) and 2-(4,4-difluorocyclohexen-1-yl)-4,4,5,5-tetramethyl-1,3,2-dioxaborolane (160 mg, 0.66 mmol) in H₂O (2 mL) and 1,4-dioxane (10 mL) was stirred at 100° C. for 16 h under N₂. The reaction mixture was diluted in aq. HCl solution (1N, 10 mL) and extracted with EtOAc (50 mL×2). The combined organic layers were dried over anhydrous Na₂SO₄ and concentrated under reduced pressure. The residue was purified by reverse-phase preparative HPLC to afford ethyl 5-(4′,4′-difluoro-2′,3′,4′,5′-tetrahydro-[1,1′-biphenyl]-4-yl)-7-oxo-4,7-dihydropyrazolo[1,5-a]pyrimidine-3-carboxylate as a white solid (110 mg, 50% yield). LCMS (ESI+) m/z 400 (M+H)⁺.

Step 3: Ethyl 5-(4-(4,4-difluorocyclohexyl)phenyl)-7-oxo-4,7-dihydropyrazolo[1,5-a]pyrimidine-3-carboxylate

A mixture of ethyl 5-(4′,4′-difluoro-2′,3′,4′,5′-tetrahydro-[1,1′-biphenyl]-4-yl)-7-oxo-4,7-dihydropyrazolo[1,5-a]pyrimidine-3-carboxylate (110 mg, 0.28 mmol) and 10% Pd on carbon (30 mg, 0.28 mmol) in EtOH (50 mL) was stirred at 15° C. for 16 h under H₂ (1 atm). The reaction mixture was filtered and concentrated under reduced pressure to afford the title compound as a white solid (39.4 mg, 35% yield). ¹H NMR (400 MHz, DMSO-d₆) □ 8.12 (s, 1H), 7.92 (d, J=8.0 Hz, 2H), 7.38 (d, J=8.0 Hz, 2H), 6.20 (s, 1H), 4.24 (q, J=7.2 Hz, 2H), 2.81-2.75 (m, 1H), 2.16-2.07 (m, 2H), 2.07-1.81 (m, 4H), 1.79-1.62 (m, 2H), 1.32 (t, J=7.2 Hz, 3H); LCMS (ESI+) m/z 402.2 (M+H)⁺.

### EXAMPLE 2

### Ethyl 5-(4-cyclopentylphenyl)-7-oxo-4,7-dihydropyrazolo[1,5-a]pyrimidine-3-carboxylate

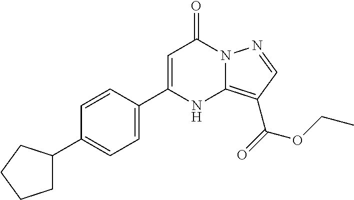 [SMILES: CCOC(=O)c1cnn2c(=O)cc(-c3ccc(C4CCCC4)cc3)[nH]c12]

The title compound was prepared according to the procedure of Example 1 using 2-cyclopentenyl-4,4,5,5-tetramethyl-1,3,2-dioxaborolane (24.6 mg, 14% yield). ¹H NMR (400 MHz, DMSO-d₆) δ 11.61 (s, 1H), 8.26 (s, 1H), 7.70 (d, J=8.0 Hz, 2H), 7.47 (d, J=8.0 Hz, 2H), 6.25 (s, 1H), 4.31 (q, J=7.2 Hz, 2H), 3.12-3.02 (m, 1H), 2.10-2.01 (m, 2H), 1.84-1.54 (m, 6H), 1.34 (t, J=7.2 Hz, 3H); LCMS (ESI+) m/z 352.2 (M+H)⁺.

### EXAMPLE 3

### 5-(4-Cyclopentylphenyl)-N,N-dimethyl-7-oxo-4,7-dihydropyrazolo[1,5-a]pyrimidine-3-carboxamide

The reaction scheme was as follows:

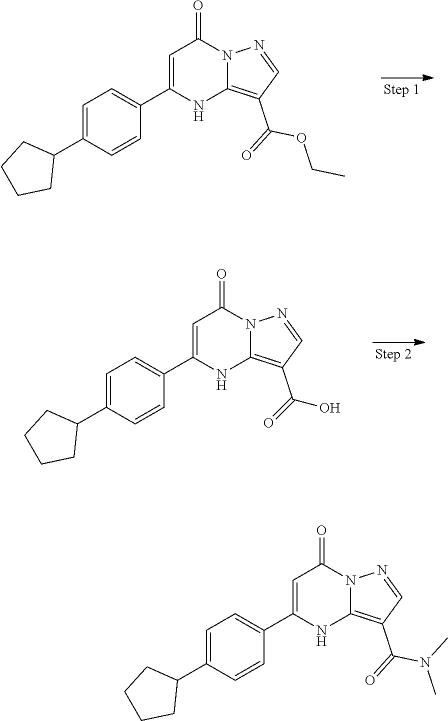 [SMILES: CCOC(=O)c1cnn2c(=O)cc(-c3ccc(C4CCCC4)cc3)[nH]c12 ; O=C(O)c1cnn2c(=O)cc(-c3ccc(C4CCCC4)cc3)[nH]c12 ; CN(C)C(=O)c1cnn2c(=O)cc(-c3ccc(C4CCCC4)cc3)[nH]c12]

Step 1: 5-(4-Cyclopentylphenyl)-7-oxo-4,7-dihydropyrazolo[1,5-a]pyrimidine-3-carboxylic acid

A mixture of ethyl 5-(4-cyclopentylphenyl)-7-oxo-4,7-dihydropyrazolo[1,5-a]pyrimidine-3-carboxylate from example 2, (180 mg, 0.51 mmol), and lithium hydroxide monohydrate (76 mg, 1.8 mmol) in H₂O (3 mL) and EtOH (3 mL) was stirred at 75° C. for 16 h. The volatile solvent was removed under reduced pressure, and the reaction mixture was adjusted to pH 7 with 2 N HCl. The resulting precipitate was filtered to give 5-(4-cyclopentylphenyl)-7-oxo-4,7-dihydropyrazolo[1,5-a]pyrimidine-3-carboxylic acid as a white solid (150 mg, 90% yield); LCMS (ESI+) m/z 324 (M+H)⁺.

Step 2: 5-(4-Cyclopentylphenyl)-N,N-dimethyl-7-oxo-4,7-dihydropyrazolo[1,5-a]pyrimidine-3-carboxamide

To a solution of 5-(4-cyclopentylphenyl)-7-oxo-4,7-dihydropyrazolo[1,5-a]pyrimidine-3-carboxylic acid (150 mg, 0.46 mmol), N,N-diisopropylethylamine (0.23 mL, 1.39 mmol) and N,N-dimethylamine hydrochloride (57 mg, 0.70 mmol) in DMF (5 mL) was added HATU (0.26 g, 0.70 mmol), and the reaction mixture was stirred at 20° C. for 16 h. The reaction mixture was concentrated under reduced pressure, and the crude was purified by reverse-phase preparative HPLC to give the title compound as a white solid (84 mg, 50% yield). ¹H NMR (400 MHz, DMSO-d₆) δ 11.80 (s, 1H), 8.27 (s, 1H), 7.74 (d, J=8.0 Hz, 2H), 7.48 (d, J=8.0 Hz, 2H), 6.27 (s, 1H), 3.28-3.02 (m, 7H), 2.12-2.01 (m, 2H), 1.85-1.80 (m, 2H), 1.71-1.64 (m, 2H), 1.63-1.54 (m, 2H); LCMS (ESI+) m/z 351.0 (M+H)³⁰.

### EXAMPLE 4

### Ethyl 5-(4-cyclohexylphenyl)-7-oxo-4,7-dihydropyrazolo[1,5-a]pyrimidine-3-carboxylate

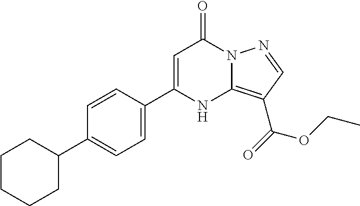 [SMILES: CCOC(=O)c1cnn2c(=O)cc(-c3ccc(C4CCCCC4)cc3)[nH]c12]

The title compound was prepared according to the procedure of Example 1 using 2-cyclohexene-1-boronic acid pinacol ester (63.2 mg, 32% yield). ¹H NMR (400 MHz, DMSO-d₆) δ 7.99 (s, 1H), 7.98 (d, J=8.0 Hz, 2H), 7.27 (d, J=8.0 Hz, 2H), 6.09 (s, 1H), 4.18 (q, J=7.2 Hz, 2H), 2.54-2.49 (m, 1H), 1.82-1.73 (m, 4H), 1.70-1.69 (m, 1H), 1.46-1.36 (m, 5H), 1.29 (t, J=7.2 Hz, 3H); LCMS (ESI+) m/z 366.2 (M+H)⁺.

### EXAMPLE 5

5-(4-cyclohexylphenyl)-N-(2-hydroxyethyl)-N-methyl-7-oxo-4,7-dihydropyrazolo[1,5-a]pyrimidine-3-carboxamide

The reaction scheme was as follows:

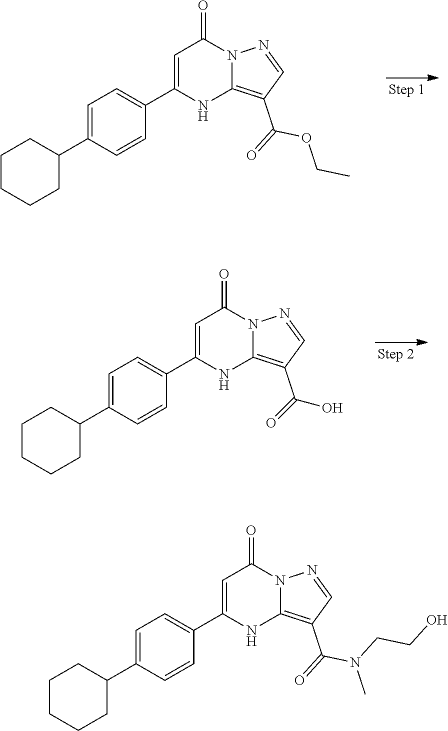 [SMILES: CCOC(=O)c1cnn2c(=O)cc(-c3ccc(C4CCCCC4)cc3)[nH]c12 ; O=C(O)c1cnn2c(=O)cc(-c3ccc(C4CCCCC4)cc3)[nH]c12 ; CN(CCO)C(=O)c1cnn2c(=O)cc(-c3ccc(C4CCCCC4)cc3)[nH]c12]

Step 1: 5-(4-cyclohexylphenyl)-7-oxo-4,7-dihydropyrazolo[1,5-a]pyrimidine-3-carboxylic acid

5-(4-Cyclohexylphenyl)-7-oxo-4,7-dihydropyrazolo[1,5-a]pyrimidine-3-carboxylic acid was prepared according to the procedure of Example 3, step 1, using ethyl 5-(4-cyclohexylphenyl)-7-oxo-4H-pyrazolo[1,5-a]pyrimidine-3-carboxylate from example 4 to obtain a yellow solid (850 mg, 97% yield). LCMS (ESI+) m/z 338.1 (M+H)⁺.

Step 2: 5-(4-Cyclohexylphenyl)-N-(2-hydroxyethyl)-N-methyl-7-oxo-4,7-dihydro pyrazolo[1,5-a]pyrimidine-3-carboxamide

The title compound was prepared according to the procedure of Example 3, step 2, using 5-(4-cyclohexylphenyl)-7-oxo-4,7-dihydropyrazolo[1,5-a]pyrimidine-3-carboxylic acid and 2-(methylamino)ethanol to obtain a white solid (7.3 mg, 4% yield). ¹H NMR (400 MHz, DMSO-d₆) δ 8.10 (s, 1H), 7.81 (d, J=8.0 Hz, 2H), 7.37 (d, J=8.0 Hz, 2H), 6.19 (s, 1H), 3.65-3.55 (m, 4H), 3.15 (s, 3H), 2.59-2.56 (m, 1H), 1.82-1.79 (m, 4H), 1.73-1.70 (m, 1H), 1.49-1.36 (m, 4H), 1.31-1.20 (m, 1H); LCMS (ESI+) m/z 395.2 (M+H)⁺.

### EXAMPLE 6

5-(4-Cyclohexylphenyl)-N-ethyl-N-methyl-7-oxo-4,7-dihydropyrazolo[1,5-a]pyrimidine-3-carboxamide

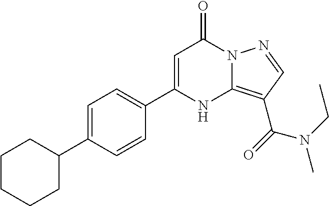 [SMILES: CCN(C)C(=O)c1cnn2c(=O)cc(-c3ccc(C4CCCCC4)cc3)[nH]c12]

The title compound was prepared according to the procedure of Example 5, using N-methylethanamine to obtain a white solid (32.1 mg, 19% yield). ¹H NMR (400 MHz, DMSO-d₆) δ 11.79 (s, 1H), 8.19 (s, 1H), 7.76 (d, J=8.0 Hz, 2H), 7.43 (d, J=8.0 Hz, 2H), 6.25 (s, 1H), 3.55-3.50 (m, 2H), 3.07 s, 3H), 2.62-2.57 (m, 1H), 1.82-1.79 (m, 4H), 1.73-1.70 (m, 1H), 1.51-1.36 (m, 4H), 1.23-1.16 (m, 4H); LCMS (ESI+) m/z 379.1 (M+H)⁺.

### EXAMPLE 7

### 5-(4-Cyclohexylphenyl)-N,N-dimethyl-7-oxo-4,7-dihydropyrazolo[1,5-a]pyrimidine-3-carboxamide

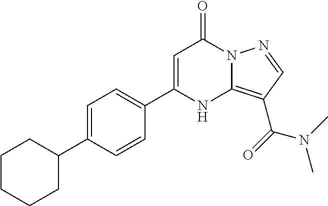 [SMILES: CN(C)C(=O)c1cnn2c(=O)cc(-c3ccc(C4CCCCC4)cc3)[nH]c12]

The title compound was prepared according to the procedure of Example 5, using dimethylamine hydrochloride to obtain a white solid (88.6 mg, 40% yield). ¹H NMR (400 MHz, DMSO-d₆) δ 11.80 (s, 1H), 8.27 (s, 1H), 7.74 (d, J=8.0 Hz, 2H), 7.46 (d, J=8.0 Hz, 2H), 6.26 (s, 1H), 3.23 (s, 3H), 3.05 (s, 3H), 2.63-2.58 (m, 1H), 1.82-1.71 (m, 5H), 1.53-1.33 (m, 4H), 1.32-1.19 (m, 1H); LCMS (ESI+) m/z 365.0 (M+H)⁺.

### EXAMPLE 8

### 3-(Azetidine-1-carbonyl)-5-(4-cyclohexylphenyl)pyrazolo[1,5-a]pyrimidin-7(4H)-one

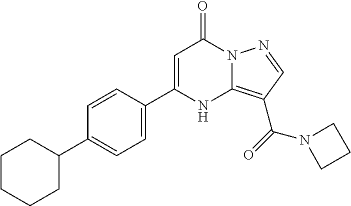 [SMILES: O=C(c1cnn2c(=O)cc(-c3ccc(C4CCCCC4)cc3)[nH]c12)N1CCC1]

The title compound was prepared according to the procedure of Example 5, using azetidine hydrochloride to obtain 4.4 mg (3% yield) of a white solid. ¹H NMR (400 MHz, DMSO-d₆) 6 11.31 (s, 1H), 8.16 (s, 1H), 7.76 (d, J=8.0 Hz, 2H), 7.44 (d, J=8.0 Hz, 2H), 6.29 (s, 1H), 4.49 (s, 2H), 4.06 (s, 2H), 2.63-2.60 (m, 1H), 2.33 (s, 2H), 1.82-1.79 (m, 4H), 1.73-1.70 (m, 1H), 1.49-1.33 (m, 4H), 1.28-1.23 (m, 1H); LCMS (ESI+) m/z 377.1 (M+H)⁺.

### EXAMPLE 9

### 5-(4-Cyclohexylphenyl)-3-(3-hydroxy-3-methylazetidine-1-carbonyl)pyrazolo[1,5-a]pyrimidin-7(4H)-one

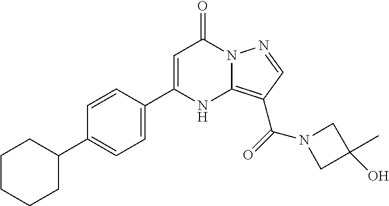 [SMILES: CC1(O)CN(C(=O)c2cnn3c(=O)cc(-c4ccc(C5CCCCC5)cc4)[nH]c23)C1]

The title compound was prepared according to the procedure of Example 5, using 3-methylazetidin-3-ol hydrochloride to obtain a white solid (51.4 mg, 21% yield). ¹H NMR (400 MHz, DMSO-d₆) δ 8.00 (s, 1H), 7.88 (d, J=8.0 Hz, 2H), 7.34 (d, J=8.0 Hz, 2H), 6.13 (s, 1H), 5.65 (s, 1H), 4.38-4.25 (m, 2H), 3.90-3.82 (m, 2H), 2.52-2.50 (m, 1H), 1.81-1.73 (m, 4H), 1.72-1.69 (m, 1H), 1.51-1.32 (m, 7H), 1.30-1.19 (m, 1H); LCMS (ESI+) m/z 407.0 (M+H)⁺.

### EXAMPLE 10

### 5-(4-Cyclohexylphenyl)-3-(3-hydroxyazetidine-1-carbonyl)pyrazolo[1,5-a]pyrimidin-7(4H)-one

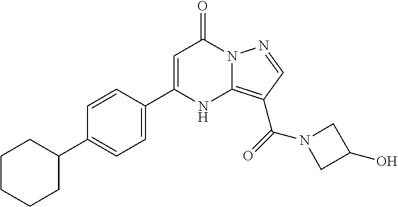 [SMILES: O=C(c1cnn2c(=O)cc(-c3ccc(C4CCCCC4)cc3)[nH]c12)N1CC(O)C1]

The title compound was prepared according to the procedure of Example 5, using azetidin-3-ol hydrochloride to obtain a white solid (44.5 mg, 19% yield). ¹H NMR (400 MHz, DMSO-d₆) δ 8.14 (s, 1H), 7.78 (d, J=8.0 Hz, 2H), 7.41 (d, J=8.0 Hz, 2H), 6.24 (s, 1H), 5.80 (s, 1H), 4.67 (s, 1H), 4.56 (s, 1H), 4.22 (s, 2H), 3.78 (s, 1H), 2.65-2.54 (m, 1H), 1.82-1.74 (m, 4H), 1.73-1.70 (m, 1H), 1.5-1.32 (m, 4H), 1.31-1.19 (m, 1H); LCMS (ESI+) m/z 393.0 (M+H)⁺.

### EXAMPLE 11

### 5-(4-Cyclohexylphenyl)-3-(3-methylazetidine-1-carbonyl)pyrazolo[1,5-a]pyrimidin-7(4H)-one

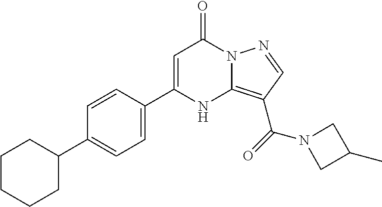 [SMILES: CC1CN(C(=O)c2cnn3c(=O)cc(-c4ccc(C5CCCCC5)cc4)[nH]c23)C1]

The title compound was prepared according to the procedure of Example 5, using 3-methylazetidine hydrochloride to obtain a white solid (22.3 mg, 9% yield). ¹H NMR (400 MHz, DMSO-d₆) δ 11.30 (s, 1H), 8.13 (s, 1H), 7.77 (d, J=8.0 Hz, 2H), 7.42 (d, J=8.0 Hz, 2H), 6.27 (s, 1H), 4.60-4.56 (m, 1H), 4.23-3.97 (m, 2H), 3.60-3.56 (m, 1H), 2.82-2.78 (m, 1H), 1.81-1.74 (m, 4H), 1.73-1.70 (m, 1H), 1.53-1.30 (m, 6H), 1.24 (d, J=6.8 Hz, 3H); LCMS (ESI+) m/z 391.1 (M+H)⁺.

### EXAMPLE 12

### 5-(4-Cyclohexylphenyl)-3-(3,3-difluoroazetidine-1-carbonyl)pyrazolo[1,5-a]pyrimidin-7(4H)-one

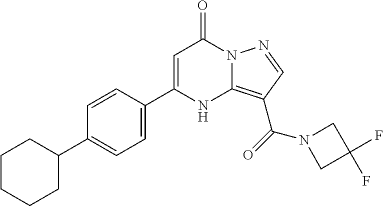 [SMILES: O=C(c1cnn2c(=O)cc(-c3ccc(C4CCCCC4)cc3)[nH]c12)N1CC(F)(F)C1]

The title compound was prepared according to the procedure of Example 5, using 3,3-difluoroazetidine hydrochloride to obtain a white solid (10.1 mg, 3% yield). ¹H NMR (400 MHz, DMSO-d₆) δ 11.34 (s, 1H), 8.22 (s, 1H), 7.73 (d, J=8.0 Hz, 2H), 7.43 (d, J=8.0 Hz, 2H), 6.31 (s, 1H), 4.75 (br s, 4H), 2.63-2.52 (m, 1H), 1.82-1.79 (m, 4H), 1.74-1.70 (m, 1H), 1.50-1.32 (m, 4H), 1.28-1.22 (m, 1H); LCMS (ESI+) m/z 413.2 (M+H)⁺.

### EXAMPLE 13

### Ethyl 5-(4-(Cyclohexyloxy)phenyl)-7-oxo-4,7-dihydropyrazolo[1,5-a]pyrimidine-3-carboxylate

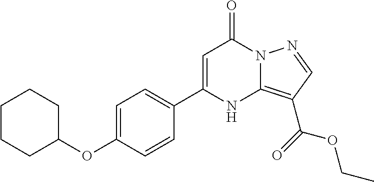 [SMILES: CCOC(=O)c1cnn2c(=O)cc(-c3ccc(OC4CCCCC4)cc3)[nH]c12]

Preparation of example 13a: Ethyl 5-chloro-7-oxo-4,7-dihydropyrazolo[1,5-a]pyrimidine-3-carboxylate

The reaction scheme was as follows:

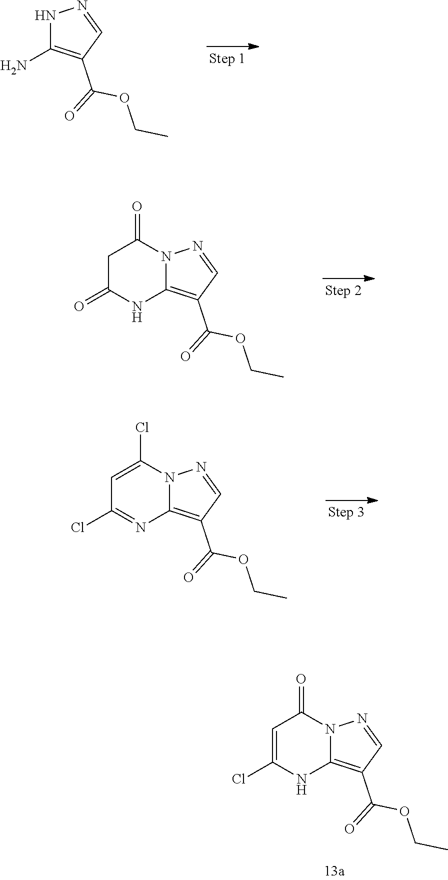 [SMILES: CCOC(=O)c1cn[nH]c1N ; CCOC(=O)c1cnn2c1NC(=O)CC2=O ; CCOC(=O)c1cnn2c(Cl)cc(Cl)nc12 ; CCOC(=O)c1cnn2c(=O)cc(Cl)[nH]c12]

Step 1: Ethyl 5,7-dioxo-4,5,6,7-tetrahydropyrazolo[1,5-a]pyrimidine-3-carboxylate

To a solution of sodium (22.2 g, 966 mmol) in EtOH (200 mL) was added ethyl 5-amino-1H-pyrazole-4-carboxylate (30.0 g, 193 mmol) and diethyl malonate (76.6 g, 580 mmol) at room temperature. The reaction solution was stirred at 100° C. for 16 h. The resulting precipitate was collected, dissolved into H₂O (1 L) and adjusted to pH 6 with 1N HCl. The precipitate was filtered and dried under vacuum to afford ethyl 5,7-dioxo-4,5,6,7-tetrahydropyrazolo[1,5-a]pyrimidine-3-carboxylate as a white solid (30 g, 69% yield). ¹H NMR (400 MHz, DMSO-d₆) δ 9.02 (s, 1H), 7.78 (s, 1H), 4.25-4.20 (m, 4H), 1.27 (t, J=7.2 Hz, 3H).

Step 2: Ethyl 5,7-dichloropyrazolo[1,5-a]pyrimidine-3-carboxylate

A mixture of ethyl 5,7-dioxo-4,5,6,7-tetrahydropyrazolo[1,5-a]pyrimidine-3-carboxylate (30 g, 134 mmol), phosphorus oxychloride (68 mL, 733 mmol) and N,N-diethylaniline (41 mL, 268 mmol) was stirred at 100° C. for 16 h. The reaction mixture was slowly poured into ice water (300 mL) and adjusted to pH 8 with saturated NaHCO₃ solution, then extracted with EtOAc (300 mL×3). The combined organic layers were washed with brine (300 mL×3), dried over anhydrous Na₂SO₄, filtered, and concentrated under reduced pressure. The crude product was purified by silica gel column chromatography (0-20% EtOAc in petroleum ether) to afford ethyl 5,7-dichloropyrazolo[1,5-a]pyrimidine-3-carboxylate as a yellow solid (30 g, 85% yield). ¹H NMR (400 MHz, DMSO-d₆) δ 8.75 (s, 1H), 7.93 (s, 1H), 4.32 (q, J=7.2 Hz, 2H), 1.32 (t, J=7.2 Hz, 3H).

Step 3: Ethyl 5-chloro-7-oxo-4,7-dihydropyrazolo[1,5-a]pyrimidine-3-carboxylate

To a solution of ethyl 5,7-dichloropyrazolo[1,5-a]pyrimidine-3-carboxylate (30 g, 115 mmol) in THF (200 mL) was added aq. NaOH solution (1M, 140 mL) at 25° C. The reaction mixture was stirred at 25° C. for 12 h. The resulting precipitate was filtered, then rinsed well with H₂O (50 mL×2) and THF (20 mL×2) to afford 13a as a white solid (6 g, 64% yield). ¹H NMR (400 MHz, DMSO-d₆) δ 8.02 (s, 1H), 5.58 (s, 1H), 4.19 (d, J=7.2 Hz, 2H), 1.25 (t, J=7.2 Hz, 3H); LCMS (ESI+) m/z 242 (M+H)⁺.

The reaction scheme was as follows:

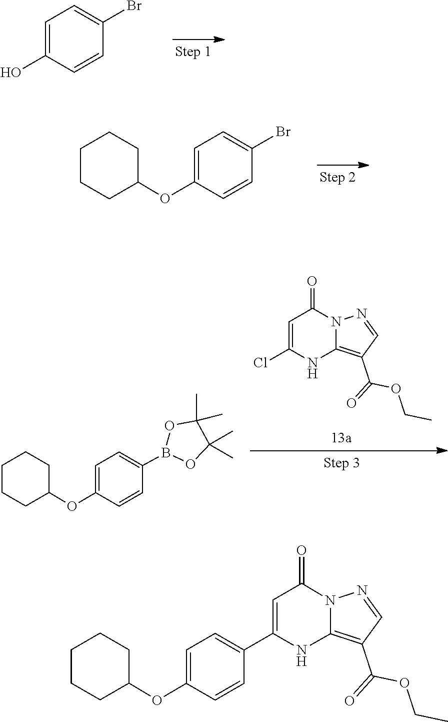 [SMILES: Oc1ccc(Br)cc1 ; Brc1ccc(OC2CCCCC2)cc1 ; CC1(C)OB(c2ccc(OC3CCCCC3)cc2)OC1(C)C ; CCOC(=O)c1cnn2c(=O)cc(Cl)[nH]c12 ; CCOC(=O)c1cnn2c(=O)cc(-c3ccc(OC4CCCCC4)cc3)[nH]c12]

Step 1: 1-Bromo-4-(cyclohexyloxy)benzene

To a solution of 4-bromophenol (2 g, 11.56 mmol), cyclohexanol (1.4 g, 14 mmol), PPh₃ (3.03 g, 11.56 mmol) in THF (6 mL) was added DIAD (2.11 mL, 11.56 mmol) at 0° C., and the reaction mixture was stirred at 15° C. for 2 h. The reaction mixture was diluted with H₂O (40 mL) and extracted with EtOAc (40 mL×2). The combined organic layers were dried over anhydrous Na₂SO₄ and concentrated under reduced pressure. The crude residue was purified by column chromatography on silica gel (0-10% ethyl acetate in petroleum ether) to give 1-bromo-4-(cyclohexyloxy)benzene as a white solid (400 mg, 14% yield). ¹H NMR (400 MHz, CDCl₃) δ 7.40-7.32 (m, 2H), 6.86-6.74 (m, 2H), 4.30-4.15 (m, 1H), 2.01-1.90 (m, 2H), 1.80-1.76 (m, 2H), 1.58-153 (m, 1H), 1.55-1.43 (m, 2H), 1.40-1.22 (m, 3H).

Step 2: 2-(4-(Cyclohexyloxy)phenyl)-4,4,5,5-tetramethyl-1,3,2-dioxaborolane

To a mixture of 1-bromo-4-(cyclohexyloxy)benzene (300 mg, 1.18 mmol), KOAc (230 mg, 2.35 mmol), bis(pinacolato)diboron (358 mg, 1.41 mmol) dissolved in 1,4-dioxane (5 mL) was added Pd(dppf)C12 (86 mg, 0.12 mmol), and the reaction mixture was stirred at 80° C. for 16 h under N₂. The reaction mixture was diluted in H₂O (50 mL) and extracted with EtOAc (50 mL×2). The combined organic layers were washed with brine (50 mL×2), dried over anhydrous Na₂SO₄ and concentrated under reduced pressure. The residue was purified by column chromatography on silica gel (0-10% ethyl acetate in petroleum ether) to afford 2-(4-(cyclohexyloxy)phenyl)-4,4,5,5-tetramethyl-1,3,2-dioxaborolane as a white solid (220 mg, 62% yield); LCMS (ESI+) m/z 303.2 (M+H)⁺.

Step 3: Ethyl 5-(4-(cyclohexyloxy)phenyl)-7-oxo-4,7-dihydropyrazolo[1,5-a]pyrimidine-3-carboxylate

To a mixture of 2-(4-(cyclohexyloxy)phenyl)-4,4,5,5-tetramethyl-1,3,2-dioxaborolane (0.22 g, 0.73 mmol), Na₂CO₃ (0.2 g, 1.86 mmol), and ethyl 5-chloro-7-oxo-4H-pyrazolo[1,5-a]pyrimidine-3-carboxylate (0.15 g, 0.62 mmol) dissolved in 1,4-dioxane (10 mL) and H₂O (1 mL) was added Pd(dppf)Cl₂ (23 mg, 0.03 mmol), and the reaction was stirred at 100° C. for 16 h under N₂. The reaction mixture was diluted with H₂O (20 mL) and extracted with EtOAc (50 mL×2). The combined organic layers were washed with brine (50 mL×2), dried over anhydrous Na₂SO₄, and concentrated under reduced pressure. The crude residue was purified by reverse-phase preparative HPLC to give the title compound as a white solid (25.1 mg, 10% yield). ¹H NMR (400 MHz, DMSO-d₆) δ 8.15 (s, 1H), 7.84 (d, J=7.6 Hz, 2H), 7.07 (d, J=8.8 Hz, 2H), 6.16 (s, 1H), 4.47 (s, 1H), 4.26 (q, J=7.2 Hz, 2H), 2.01-1.95 (m, 2H), 1.73-1.65 (m, 2H), 1.54-1.52 (m, 1H), 1.51-1.37 (m, 4H), 1.33 (t, J=7.2 Hz, 3H), 1.31-1.22 (m, 1H); LCMS (ESI+) m/z 382.3 (M+H)⁺.

### EXAMPLE 14

### Ethyl 5-(3-methyl-4-phenoxyphenyl)-7-oxo-4,7-dihydropyrazolo[1,5-a]pyrimidine-3-carboxylate

The reaction scheme was as follows:

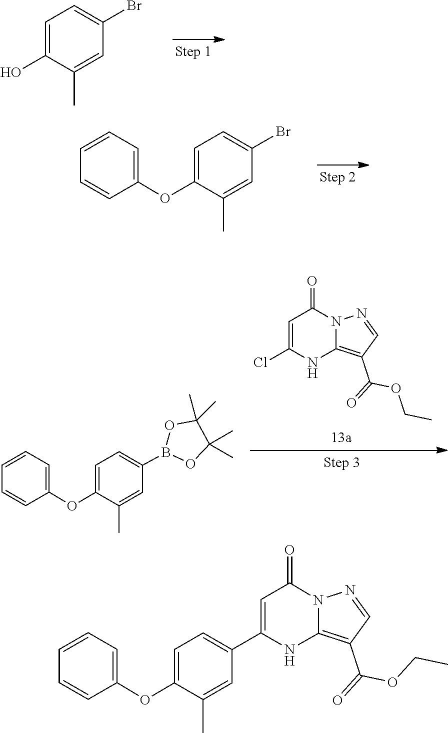

Step 1: 4-Bromo-2-methyl-1-phenoxybenzene

A mixture of phenylboronic acid (3.91 g, 32 mmol), 4-bromo-2-methylphenol (2 g, 10.69 mmol), copper (II) acetate (2.14 g, 11.76 mmol) and triethylamine (7.45 mL, 53.47 mmol) in DCM (100 mL) was stirred at 20° C. for 16 h. The mixture was filtered and the filtrate was concentrated under reduced pressure. The crude residue was purified by silica gel column chromatography (petroleum ether) to give 4-bromo-2-methyl-1-phenoxybenzene as a clear liquid (2.5 g, 88% yield). ¹H NMR (400 MHz, DMSO-d₆) δ 7.53 (d, J=2.4 Hz, 1H), 7.41-7.31 (m, 3H), 7.15-7.05 (m, 1H), 6.91 (d, J=7.6 Hz, 2H), 6.82 (d, J=8.8 Hz, 1H), 2.17 (s, 3H).

Step 2: 4,4,5,5-Tetramethyl-2-3-methyl-4-phenoxyphenyl)-1,3,2-dioxaborolane

4,4,5,5-Tetramethyl-2-(3-methyl-4-phenoxyphenyl)-1,3,2-dioxaborolane was prepared according to the procedure of Example 13, step 2, using 4-bromo-2-methyl-1-phenoxybenzene to obtain a yellow oil (900 mg, 76% yield). ¹H NMR (400 MHz, DMSO-d₆) δ 7.62 (s, 1H), 7.49 (d, J=7.6 Hz, 1H), 7.39-7.35 (m, 2H), 7.14-7.09 (m, 1H), 6.93 (d, J=8.4 Hz, 2H), 6.80 (d, J=8.4 Hz, 1H), 2.20 (s, 3H), 1.28 (s, 12H).

Step 3: Ethyl 5-(3-methyl-4-phenoxyphenyl)-7-oxo-4,7-dihydropyrazolo[1,5-a]pyrimidine-3-carboxylate

The title compound was prepared according to the procedure of Example 13, step 3, using 4,4,5,5-tetramethyl-2-(3-methyl-4-phenoxyphenyl)-1,3,2-dioxaborolane to obtain a white solid (58 mg, 24% yield). ¹H NMR (400 MHz, DMSO-d₆) δ 8.22 (s, 1H), 7.83 (s, 1H), 7.67 (d, J=8.8 Hz, 1H), 7.44-7.39 (m, 2H), 7.19-7.17 (m, 1H), 7.03-6.99 (m, 2H), 6.96 (d, J=8.4 Hz, 1H), 6.23 (s, 1H), 4.29 (q, J=7.2 Hz, 2H), 2.31 (s, 3H), 1.33 (t, J=7.2 Hz, 3H); LCMS (ESI+) m/z 390.1 (M+H)⁺.

### EXAMPLE 15

### Ethyl 7-oxo-5-(4-phenoxyphenyl)-4,7-dihydropyrazolo[1,5-a]pyrimidine-3-carboxylate

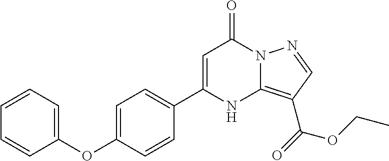 [SMILES: CCOC(=O)c1cnn2c(=O)cc(-c3ccc(Oc4ccccc4)cc3)[nH]c12]

The title compound was prepared according to the procedure of Example 13, step 3, using 4,4,5,5-tetramethyl-2-(3-methyl-4-phenoxyphenyl)-1,3,2-dioxaborolane to obtain a white solid (39.1 mg, 8% yield). ¹H NMR (400 MHz, DMSO-d₆) δ 8.23 (s, 1H), 7.86 (d, J=8.4 Hz, 2H), 7.55-7.36 (m, 2H), 7.30-7.20 (m, 1H), 7.14-7.10 (m, 4H), 6.25 (s, 1H), 4.30 (q, J=7.2 Hz, 2H), 1.33 (t, J=7.2 Hz, 3H); LCMS (ESI+) m/z 376.1 (M+H)⁺.

### EXAMPLE 16

### /VA-Dimethyl-7-oxo-5-(4-phenoxyphenyl)-4,7-dihydropyrazolo[1,5-a]pyrimidine-3-carboxamide

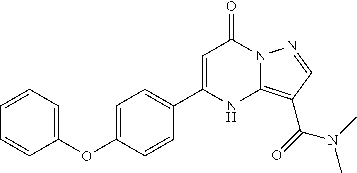 [SMILES: CN(C)C(=O)c1cnn2c(=O)cc(-c3ccc(Oc4ccccc4)cc3)[nH]c12]

The title compound was prepared according to the procedure of Example 3 using ethyl 7-oxo-5-(4-phenoxyphenyl)-4H-pyrazolo[1,5-a]pyrimidine-3-carboxylate from example 15 to obtain a white solid (197.5 mg, 91% yield). ¹H NMR (400 MHz, CD₃OD) δ 8.27 (s, 1H), 7.84 (d, J=8.8 Hz, 2H), 7.51-7.37 (m, 2H), 7.28-7.19 (m, 1H), 7.15-7.09 (m, 4H), 6.27 (s, 1H), 3.35 (s, 3H), 3.14 (s, 3H); LCMS (ESI) m/z 375.2 (M+H)⁺.

### EXAMPLE 17

### N-Ethyl-7-oxo-5-(4-phenoxyphenyl)-4,7-dihydropyrazolo[1,5-a]pyrimidine-3-carboxamide

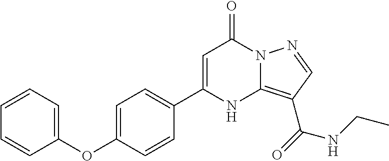 [SMILES: CCNC(=O)c1cnn2c(=O)cc(-c3ccc(Oc4ccccc4)cc3)[nH]c12]

The title compound was prepared according to the procedure of Example 16 using ethylamine hydrochloride to obtain a white solid (186.7 mg, 86% yield). ¹H NMR (400 MHz, CD₃OD) δ 8.21 (s, 1H), 8.10-7.89 (m, 2H), 7.50-7.25 (m, 2H), 7.23-7.11 (m, 1H), 7.08-6.94 (m, 4H), 6.31 (s, 1H), 3.47 (q, J=7.2 Hz, 2H), 1.29 (t, J=7.2 Hz, 3H); LCMS (ESI+) m/z 375.1 (M+H)⁺.

### EXAMPLE 18

### Ethyl 7-oxo-5-(4-(piperidin-1-yl)phenyl)-4,7-dihydropyrazolo[1,5-a]pyrimidine-3-carboxylate

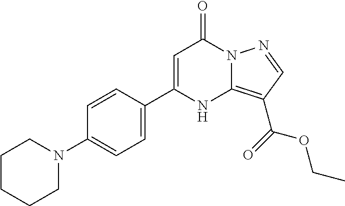 [SMILES: CCOC(=O)c1cnn2c(=O)cc(-c3ccc(N4CCCCC4)cc3)[nH]c12]

The title compound was prepared according to the procedure of Example 13, step 3, using 1-(4-(4,4,5,5-tetramethyl-1,3,2-dioxaborolan-2-yl)phenyppiperidine to obtain a white solid (1.7 mg, 2.2% yield). LCMS (ESI+) m/z 367.1 (M+H)⁺.

### EXAMPLES 19 and 20

### (S)-Ethyl5-(4-(2,2-dimethylcyclopentyl)phenyl)-7-oxo-4,7-dihydropyrazolo[1,5-a]pyrimidine-3-carboxylate and

### (R)-ethyl 5-(4-(2,2-dimethylcyclopentyl)phenyl)-7-oxo-4,7-dihydropyrazolo[1,5-a]pyrimidine-3-carboxylate, arbitrarily assigned

The reaction scheme was as follows:

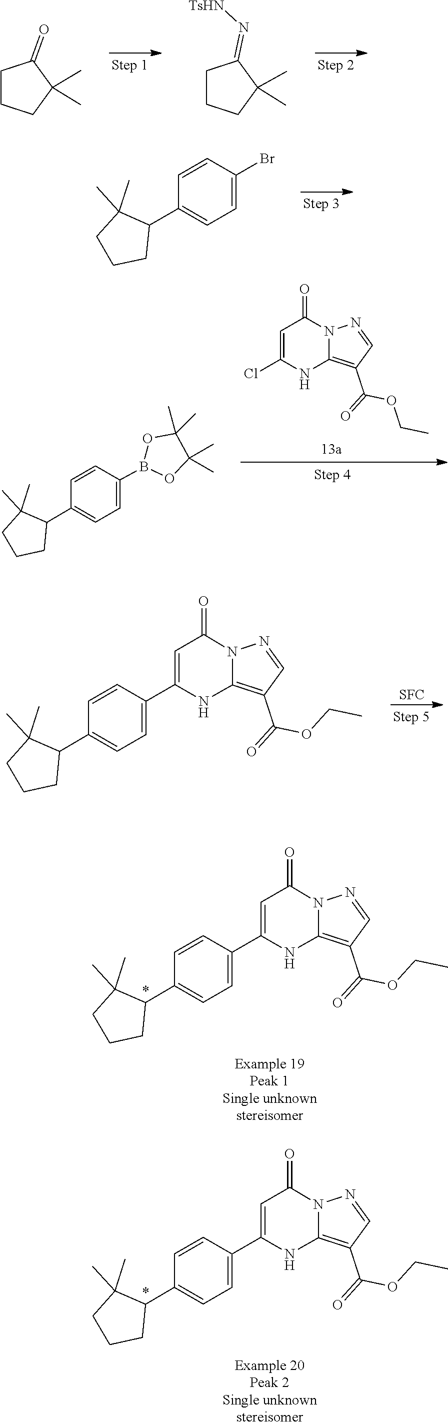

Step 1: N-(2,2-Dimethylcyclopentylidene)-4-methylbenzenesulfonohydrazide

To a solution of 2,2-dimethylcyclopentanone (2.36 g, 21 mmol) in MeOH (50 mL) was added 4-methyl phenylsulfonohydrazide (3.92 g, 21 mmol) at 25° C., and the reaction mixture was stirred at 80° C. for 16 h. The reaction mixture was concentrated under reduced pressure to give crude N-(2,2-dimethylcyclopentylidene)-4-methylbenzenesulfono-hydrazide (5.6 g, 94% yield), which was used directly without further purification. LCMS (ESI+) m/z 281.0 (M+H)⁺.

Step 2: 1-Bromo-4-(2,2-dimethylcyclopentyl)benzene

To a solution of N-(2,2-dimethylcyclopentylidene)-4-methylbenzenesulfono-hydrazide (5.4 g, 19 mmol) in 1,4-dioxane (200 mL) was added (4-bromophenyl)boronic acid (5.8 g, 28 mmol) and CsCO₃ (12.54 g, 38 mmol) under N₂. The reaction mixture was stirred at 110° C. for 16 h and then concentrated under reduced pressure. The crude residue was purified by silica gel column chromatography (petroleum ether) to give 1-bromo-4-(2,2-dimethylcyclopentyl)benzene (1 g, 20% yield) as a colorless oil.

Step 3: 2-[4-(2,2-Dimethylcyclopentyl)phenyl]-4,4,5,5-tetramethyl-1,3,2-dioxaborolane.

2-[4-(2,2-Dimethylcyclopentyl)phenyl]-4,4,5,5-tetramethyl-1,3,2-dioxaborolane was prepared according to the procedure of Example 13, step 2, using 1-bromo-4-(2,2-dimethylcyclopentyl)benzene to obtain 2-[4-(2,2-dimethylcyclopentyl)phenyl]-4,4,5,5-tetramethyl-1,3,2-dioxaborolane (700 mg, 59% yield) as a white solid. ¹H NMR (400 MHz, CDCl₃) δ 7.73 (d, J=8.0 Hz, 2H), 7.21 (d, J=8.0 Hz, 2H), 2.74-2.69 (m, 1H), 2.16-2.07 (m, 1H), 2.04-1.95 (m, 1H), 1.88-1.67 (m, 2H), 1.66-1.58 (m, 2H), 1.36 (s, 12H), 0.99 (s, 3H), 0.62 (s, 3H).

Step 4: Ethyl 5-(4-(2,2-dimethylcyclopentyl)phenyl)-7-oxo-4,7-dihydropyrazolo[1,5-a]pyrimidine-3-carboxylate

Ethyl 5-(4-(2,2-dimethylcyclopentyl)phenyl)-7-oxo-4,7-dihydropyrazolo[1,5-a]pyrimidine-3-carboxylate was prepared according to the procedure of Example 13, step 3, using 2-[4-(2,2-dimethylcyclopentyl)phenyl]-4,4,5,5-tetramethyl-1,3,2-dioxaborolane to obtain a white solid (300 mg, 59% yield). LCMS (ESI+) m/z 380.1 (M+H)⁺.

Step 5: Chiral separation of ethyl 5-(4-(2,2-dimethylcyclopentyl)phenyl)-7-oxo-4,7-dihydropyrazolo[1,5-a]pyrimidine-3-carboxylate

Racemic ethyl 5-(4-(2,2-dimethylcyclopentyl)phenyl)-7-oxo-4,7-dihydropyrazolo[1,5-a]pyrimidine-3-carboxylate (60 mg, 0.16 mmol) was separated by SFC (YMC Chiral Amylose-c, isocratic 55% EtOH w/0.1% NH₄OH, 38° C., 5.5 min) to give 20.3 mg (33.8% yield) of enantiomer 1. Chiral SFC Peak 1 (RT=4.598 min), % ee=99; ¹H NMR (400 MHz, CD₃OD) δ 8.25 (s, 1H), 7.78 (d, J=8.4 Hz, 2H), 7.44 (d, J=8.4 Hz, 2H), 6.29 (s, 1H), 4.39 (q, J=7.2 Hz, 2H), 2.87-2.82 (m, 1H), 2.26-2.14 (m, 1H), 2.10-2.00 (m, 1H), 1.96-1.74 (m, 2H), 1.70-1.64 (m, 2H), 1.41 (t, J=7.2 Hz, 3H), 1.04 (s, 3H), 0.68 (s, 3H); LCMS (ESI+) m/z 380.1 (M+H)⁺.

The second peak was collected to give 20.2 mg (33.7% yield) of enantiomer 2. Chiral SFC Peak 2 (RT=8.175 min), % ee=99; ¹H NMR (400 MHz, CD₃OD) δ 8.25 (s, 1H), 7.77 (d, J=8.4 Hz, 2H), 7.44 (d, J=8.4 Hz, 2H), 6.29 (s, 1H), 4.39 (q, J=7.2 Hz, 2H), 2.89-2.81 (m, 1H), 2.26-2.14 (m, 1H), 2.10-2.00 (m, 1H), 1.96-1.85 (m, 1H), 1.83-1.73 (m, 1H), 1.70-1.63 (m, 2H), 1.41 (t, J=7.2 Hz, 3H), 1.04 (s, 3H), 0.68 (s, 3H); LCMS (ESI+) m/z 380.1 (M+H)⁺.

### EXAMPLES 21 and 22

### (R)-5-(4(2,2-Dimethylcyclopentyl)phenyl)-N,N-dimethyl-7-oxo-4,7-dihydropyrazolo[1,5-a]pyrimidine-3-carboxamide and

### (S)-5-(4-(2,2-dimethylcyclopentyl)phenyl)-N,N-dimethyl-7-oxo-4,7-dihydropyrazolo[1,5-a]pyrimidine-3-carboxamide, arbitrarily assigned

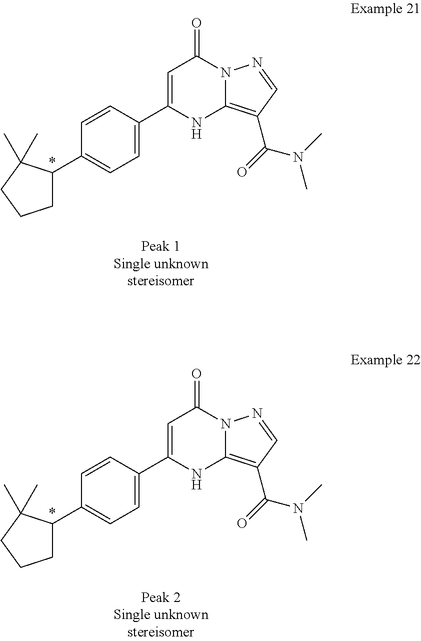 [SMILES: CN(C)C(=O)c1cnn2c(=O)cc(-c3ccc(C4CCCC4(C)C)cc3)[nH]c12 ; CN(C)C(=O)c1cnn2c(=O)cc(-c3ccc(C4CCCC4(C)C)cc3)[nH]c12]

Step 1: 5-(4-(2,2-Dimethylcyclopentyl)phenyl)-N,N-dimethyl-7-oxo-4,7-dihydropyrazolo[1,5-a]pyrimidine-3-carboxamide

Racemic 5-(4-(2,2-Dimethylcyclopentyl)phenyl)-N,N-dimethyl-7-oxo-4,7-dihydropyrazolo[1,5-a]pyrimidine-3-carboxamide was prepared according to the procedure of Example 3, using ethyl 5-(4-(2,2-dimethylcyclopentyl)phenyl)-7-oxo-4,7-dihydropyrazolo[1,5-a]pyrimidine-3-carboxylate to obtain 40 mg (44.2% over two steps) of a white solid. LCMS (ESI+) m/z 379.1 (M+H)⁺.

Step 2: Chiral separation of 5-(4-(2,2-dimethylcyclopentyl)phenyl)-N,N-dimethyl-7-oxo-4,7-dihydropyrazolo[1,5-a]pyrimidine-3-carboxamide

Racemic 5-(4-(2,2-dimethylcyclopentyl)phenyl)-N,N-dimethyl-7-oxo-4,7-dihydropyrazolo[1,5-a]pyrimidine-3-carboxamide (40 mg, 0.11 mmol) was separated by SFC (YMC Chiral Amylose-c, isocratic 55% EtOH w/0.1% NH₄OH, 38° C., 9.9 min) to give 5.1 mg (12.8% yield) of enantiomer 1. Chiral SFC Peak 1 (RT=4.390 min), % ee=99; ¹H NMR (400 MHz, CD₃OD) δ 8.27 (s, 1H), 7.79 (d, J=7.6 Hz, 2H), 7.44 (d, J=7.6 Hz, 2H), 6.31 (s, 1H), 3.37 (s, 3H), 3.15 (s, 3H), 2.89-2.80 (m, 1H), 2.27-2.14 (m, 1H), 2.11-2.00 (m, 1H), 1.94-1.84 (m, 1H), 1.83-1.72 (m, 1H), 1.70-1.62 (m, 2H), 1.04 (s, 3H), 0.68 (s, 3H); LCMS (ESI+) m/z 379.1 (M+H)⁺.

The second peak was collected to give 5.1 mg (12.8% yield) of enantiomer 2. Chiral SFC Peak 2 (RT=7.343 min), % ee=99; ¹H NMR (400 MHz, CD₃OD) δ 8.24 (s, 1H), 7.81 (d, J=8.0 Hz, 2H), 7.41 (d, J=7.6 Hz, 2H), 6.31 (s, 1H), 3.36 (s, 3H), 3.15 (s, 3H), 2.87-2.81 (m, 1H), 2.26-2.13 (m, 1H), 2.10-2.00 (m, 1H), 1.96-1.84 (m, 1H), 1.82-1.71 (m, 1H), 1.70-1.62 (m, 2H), 1.04 (s, 3H), 0.68 (s, 3H); LCMS (ESI+) m/z 379.1 (M+H)⁺.

### EXAMPLE 23

### Ethyl 7-oxo-5-(4-(trifluoromethyl)phenyl)-4,7-dihydropyrazolo[1,5-a]pyrimidine-3-carboxylate

The reaction scheme was as follows:

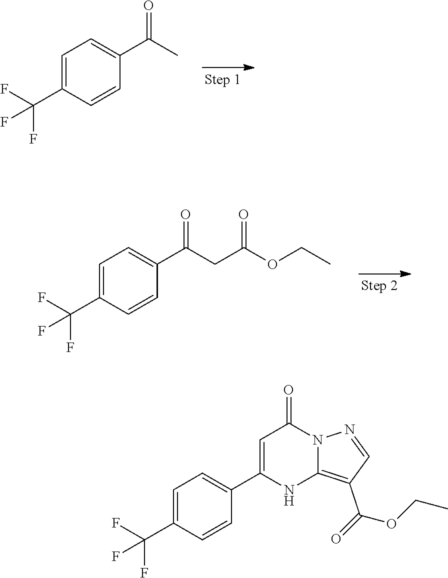 [SMILES: CC(=O)c1ccc(C(F)(F)F)cc1 ; CCOC(=O)CC(=O)c1ccc(C(F)(F)F)cc1 ; CCOC(=O)c1cnn2c(=O)cc(-c3ccc(C(F)(F)F)cc3)[nH]c12]

Step 1: Ethyl 3-oxo-3-(4-(trifluoromethyl)phenyl)propanoate

To a solution of 4′-(trifluoromethyl)acetophenone (8.0 g, 43 mmol) in toluene (15 mL) was added NaH (2.5 g, 64 mmol, 60% in mineral oil) and stirred for 30 min at 0° C. Diethyl carbonate (10 g, 85 mmol) was added, and the reaction mixture was stirred at 15° C. for 3 h. The reaction mixture was diluted with H₂O (30 mL) and extracted with EtOAc (50 mL×3). The combined organic layers were dried over anhydrous Na₂SO₄, filtered, and concentrated under reduced pressure. The crude residue was purified by silica gel column chromatography (0-10% EtOAc in petroleum ether) to give ethyl 3-oxo-3-(4-(trifluoromethyl)phenyl)propanoate as a yellow oil (5.7 g, 51% yield); LCMS (ESI+): m/z 261 (M+H)⁺.

Step 2: Ethyl 7-oxo-5-(4-(trifluoromethyl)phenyl)-4,7-dihydropyrazolo[1,5-a]pyrimidine-3-carboxylate

A mixture of ethyl 3-oxo-3-(4-(trifluoromethyl)phenyl)propanoate (200 mg, 0.77 mmol) and ethyl 5-amino-1H-pyrazole-4-carboxylate (143 mg, 0.92 mmol) in AcOH (2 mL) was stirred at 110° C. for 5 h. The reaction mixture was diluted with EtOAc (40 mL). The organic layer was washed with water (40 mL×2), dried over anhydrous Na₂SO₄, filtered, and concentrated under reduced pressure. The crude residue was purified by reverse-phase preparative HPLC to give the title compound as a white solid (42.1 mg, 15% yield). ¹H NMR (400 MHz, CD₃OD) δ 8.29 (s, 1H), 8.21 (d, J=7.2 Hz, 2H), 7.79 (d, J=8.4 Hz, 2H), 6.44 (s, 1H), 4.36 (q, J=7.2 Hz, 2H), 1.41 (t, J=7.2 Hz, 3H); LCMS (ESI) m/z 352 (M+H)⁺.

### EXAMPLE 24

### N-Ethyl-7-oxo-5-(4-(trifluoromethyl)phenyl)-4,7-dihydropyrazolo[1,5-a]pyrimidine-3-carboxamide

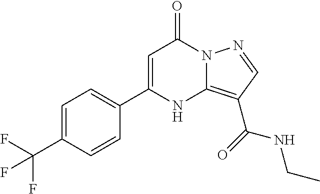 [SMILES: CCNC(=O)c1cnn2c(=O)cc(-c3ccc(C(F)(F)F)cc3)[nH]c12]

A mixture of ethyl 7-oxo-5-(4-(trifluoromethyl)phenyl)-4,7-dihydropyrazolo[1,5-a]pyrimidine-3-carboxylate from example 23 (200 mg, 0.57 mmol) and ethylamine (5 mL, 0.57 mmol) in EtOH (3 mL) was stirred at 100° C. in a sealed tube for 36 h. The solution was concentrated under reduced pressure, and the resulting residue was purified by reverse-phase preparative HPLC to afford the title compound as a white solid (39 mg, 18% yield). ¹H NMR (400 MHz, DMSO-d₆) δ 11.62 (s, 1H), 8.48 (s, 1H), 8.40 (s, 1H), 8.05-8.00 (m, 2H), 7.91 (d, J=7.2 Hz, 2H), 6.32 (s, 1H), 3.32-3.30 (m, 2H), 1.15 (t, J=7.2 Hz, 3H); LCMS (ESI+) m/z 351.0 (M+H)⁺.

### EXAMPLE 25

### N-methyl-7-oxo-5-(4-(trifluoromethyl)phenyl)-4,7-dihydropyrazolo[1,5-a]pyrimidine-3-carboxamide

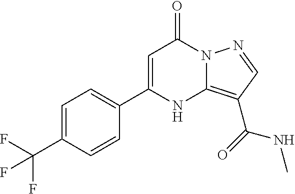 [SMILES: CNC(=O)c1cnn2c(=O)cc(-c3ccc(C(F)(F)F)cc3)[nH]c12]

The title compound was prepared according to the procedure of Example 24 using methanamine in EtOH to obtain a white solid (23 mg, 24% yield). ¹H NMR (400 MHz, DMSO-d₆) δ 11.59 (s, 1H), 8.43 (d, J=4.4 Hz, 1H), 8.35 (s, 1H), 8.02 (s, 2H), 7.94 (d, J=7.6 Hz, 2H), 6.31 (s, 1H), 2.81 (d, J=4.4 Hz, 3H); LCMS (ESI+) m/z 337.0 (M+H)⁺.

### EXAMPLE 26

### Ethyl 5-(3-methyl-4-(trifluoromethyl)phenyl)-7-oxo-4,7-dihydropyrazolo[1,5-a]pyrimidine-3-carboxylate

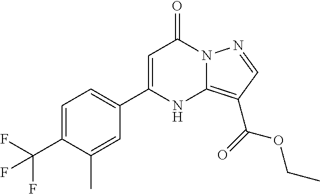 [SMILES: CCOC(=O)c1cnn2c(=O)cc(-c3ccc(C(F)(F)F)c(C)c3)[nH]c12]

The title compound was prepared according to the procedure of Example 13, step 3, using (3-methyl-4-(trifluoromethyl)phenyl)boronic acid to obtain a white solid (25.7 mg, 13% yield). ¹H NMR (400 MHz, DMSO-d₆) δ 11.96 (s, 1H), 8.28 (s, 1H), 7.87 (d, J=8.0 Hz, 2H), 7.77 (d, J=8.0 Hz, 1H), 6.32 (s, 1H), 4.32 (q, J=7.2 Hz, 2H), 2.55 (s, 3H), 1.34 (t, J=7.2 Hz, 3H); LCMS (ESI+) m/z 366.0 (M+H)⁺.

### EXAMPLE 27

### N-Cyclopropyl-7-oxo-5-(4-(trifluoromethyl)phenyl)-4,7-dihydropyrazolo[1,5-a]pyrimidine-3-carboxamide

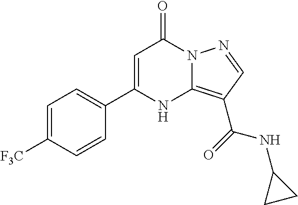 [SMILES: O=C(NC1CC1)c1cnn2c(=O)cc(-c3ccc(C(F)(F)F)cc3)[nH]c12]

The title compound was prepared according to the procedure of Example 3 using ethyl 7-oxo-5-(4-(trifluoromethyl)phenyl)-4,7-dihydropyrazolo[1,5-a]pyrimidine-3-carboxylate from example 23 in place of ethyl 5-(4-cyclopentylphenyl)-7-oxo-4,7-dihydropyrazolo[1,5-a]pyrimidine-3-carboxylate and cyclopropylamine in place of N,N-dimethylamine hydrochloride to obtain a white solid (8.1 mg, 10% yield over two-steps). ¹H NMR (400 MHz, CD₃OD) δ 8.26 (s, 1H), 8.18 (d, J=8.0 Hz, 2H), 7.79 (d, J=8.0 Hz, 2H), 6.39 (s, 1H), 2.92-2.87 (m, 1H), 0.91-0.86 (m, 2H), 0.68-0.64 (m, 2H); LCMS (ESI+) m/z 362.9 (M+H)⁺, 384.9 (M+Na)⁺.

### EXAMPLE 28

### Ethyl 2-methyl-7-oxo-5-(4-phenoxyphenyl)-4,7-dihydropyrazolo[1,5-a]pyrimidine-3-carboxylate

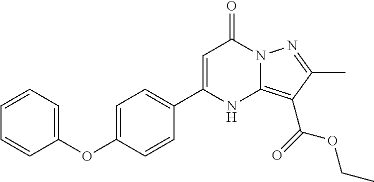 [SMILES: CCOC(=O)c1c(C)nn2c(=O)cc(-c3ccc(Oc4ccccc4)cc3)[nH]c12]

The title compound was prepared according to the procedure of Example 23, using 1-(4-phenoxyphenyl)ethan-1-one in place of 4′-(trifluoromethyl)acetophenone and ethyl 5-amino-3-methyl-1H-pyrazole-4-carboxylate in place of ethyl 5-amino-1H-pyrazole-4-carboxylate, to obtain a white solid (48.7 mg, 2.4% yield over two steps). ¹H NMR (400 MHz, CDCl₃) δ 7.71-7.56 (m, 2H), 7.49-7.37 (m, 2H), 7.26-7.20 (m, 1H), 7.17-7.02 (m, 4H), 6.21 (s, 1H), 4.41 (q, J=7.2 Hz, 2H), 2.61 (s, 3H), 1.44 (t, J=7.2 Hz, 3H); LCMS (ESI+) m/z 390.2 (M+H)⁺.

### EXAMPLE 29

### Ethyl 2-isopropyl-7-oxo-5-(4-phenoxyphenyl)-4,7-dihydropyrazolo[1,5-a]pyrimidine-3-carboxylate

The reaction scheme was as follows:

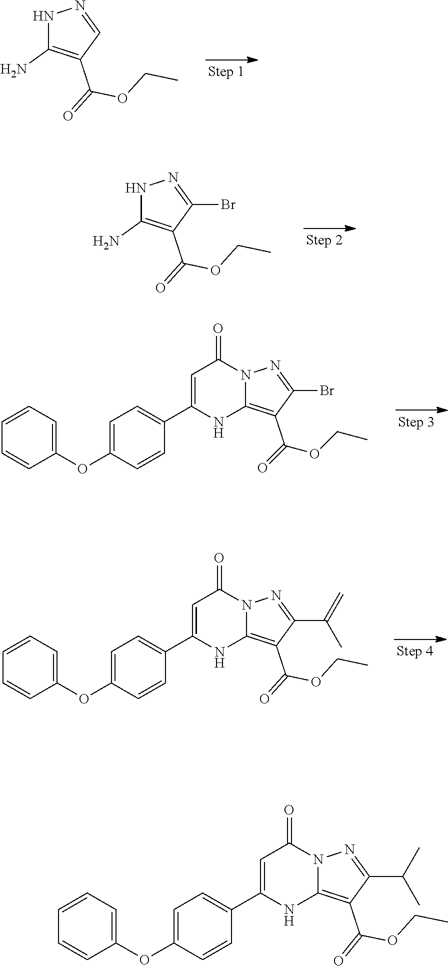 [SMILES: CCOC(=O)c1cn[nH]c1N ; CCOC(=O)c1c(Br)n[nH]c1N ; CCOC(=O)c1c(Br)nn2c(=O)cc(-c3ccc(Oc4ccccc4)cc3)[nH]c12 ; C=C(C)c1nn2c(=O)cc(-c3ccc(Oc4ccccc4)cc3)[nH]c2c1C(=O)OCC ; CCOC(=O)c1c(C(C)C)nn2c(=O)cc(-c3ccc(Oc4ccccc4)cc3)[nH]c12]

Step 1: Ethyl 5-amino-3-bromo-1H-pyrazole-4-carboxylate

To a solution of ethyl 5-amino-1H-pyrazole-4-carboxylate (2.0 g, 12.9 mmol) in acetonitrile (20 mL) was added NBS (2.5 g, 14 mmol) slowly at 15° C., and the reaction mixture was stirred at RT for 3 h. The reaction mixture was quenched with saturated aqueous Na₂SO₃ solution (50 mL) and extracted with EtOAc (50 mL). The combined organic layers were washed with brine (50 mL×2), filtered, dried over Na₂SO₄, and concentrated under reduced pressure to give ethyl 5-amino-3-bromo-1H-pyrazole-4-carboxylate as a yellow solid (2.7 g, 89% yield). LCMS (ESI+) m/z 234.0 (M+H)⁺.

Step 2: Ethyl 2-bromo-7-oxo-5-(4-phenoxyphenyl)-4,7-dihydropyrazolo[1,5-a]pyrimidine-3-carboxylate

A mixture of ethyl 3-oxo-3-(4-phenoxyphenyl)propanoate (4.0 g, 13.9 mmol),p-TsOH.H₂O (600 mg, 3.2 mmol) and ethyl 5-amino-3-bromo-1H-pyrazole-4-carboxylate (2.7 g, 11.5 mmol) in n-butanol (30 mL) was stirred at 130° C. for 8 h. The reaction mixture was diluted with EtOAc (400 mL) and washed with saturated NaHCO₃ solution (100 mL×2). The combined organic layers were dried over anhydrous Na₂SO₄, filtered, and concentrated under reduced pressure. The crude residue was purified by silica gel column chromatography (0-50% EtOAc in petroleum ether) to afford ethyl 2-bromo-7-oxo-5-(4-phenoxyphenyl)-4,7-dihydropyrazolo[1,5-a]pyrimidine-3-carboxylate as a yellow solid (0.8 g, 15% yield). ¹H NMR (400 MHz, CDCl₃) δ 9.95 (s, 1H), 7.61 (d, J=8.8 Hz, 2H), 7.45-7.36 (m, 2H), 7.24-7.18 (m, 1H), 7.13-7.06 (m, 4H), 6.20 (d, J=2.4 Hz, 1H), 4.44-4.38 (q, J=7.2 Hz, 2H), 1.47-1.39 (t, J=7.2 Hz, 3H).

Step 3: Ethyl 7-oxo-5-(4-phenoxyphenyl)-2-(prop-1-en-2-yl)-4,7-dihydropyrazolo[1,5-a]pyrimidine-3-carboxylate

A mixture of ethyl 2-bromo-7-oxo-5-(4-phenoxyphenyl)-4,7-dihydropyrazolo[1,5-a]pyrimidine-3-carboxylate (50 mg, 0.11 mmol), Na₂CO₃ (35 mg, 0.33 mmol), isopropenyl boronic acid pinacol ester (36 mg, 0.22 mmol) and Pd(dppf)Cl₂ (4 mg, 0.01 mmol) in 1,4-dioxane (3 mL) and H₂O (0.30 mL) was stirred at 100° C. for 16 h under N₂. The reaction solution was adjusted to pH 7 with 1 N HCl and then extracted with EtOAc (50 mL×2). The combined organic layers were washed with brine (30 mL), dried over anhydrous Na₂SO₄, filtered, and concentrated under reduced pressure to give a solid, which was then purified by preparative TLC (50% EtOAc in petroleum ether) to give ethyl 7-oxo-5-(4-phenoxyphenyl)-2-(prop-1-en-2-yl)-4,7-dihydropyrazolo[1,5-a]pyrimidine-3-carboxylate as a white solid (35 mg, 76% yield). LCMS (ESI+) m/z 415.9 (M+H)⁺.

Step 4: Ethyl 2-isopropyl-7-oxo-5-(4-phenoxyphenyl)-4,7-dihydropyrazolo[1,5-a]pyrimidine-3-carboxylate

A mixture of ethyl 7-oxo-5-(4-phenoxyphenyl)-2-(prop-1-en-2-yl)-4,7-dihydropyrazolo[1,5-a]pyrimidine-3-carboxylate (40 mg, 0.10 mmol) and 10% Pd on carbon (10 mg) in MeOH (50 mL) was stirred for 6 h under H₂ (1 atm). The reaction solution was filtered and evaporated in vacuo to dryness to give the title compound as a white solid (17 mg, 41% yield). ¹H NMR (400 MHz, DMSO-d₆) δ 11.12 (s, 1H), 7.84 (d, J=8.4 Hz, 2H), 7.50-7.44 (m, 2H), 7.24 (t, J=7.6 Hz, 1H), 7.16-7.11 (m, 4H), 6.22 (s, 1H), 4.30 (q, J=7.2 Hz, 2H), 3.58-3.51 (m, 1H), 1.38-1.33 (m, 3H), 1.30-1.28 (m, 6H); LCMS (ESI+) m/z 418.1 (M+H)⁺.

### EXAMPLE 30

### Ethyl 5-(4-(tert-butyfiphenyl)-7-oxo-4,7-dihydropyrazolo[1,5-a]pyrimidine-3-carboxylate

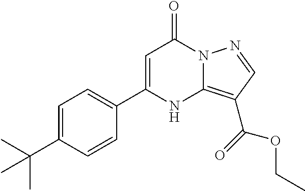 [SMILES: CCOC(=O)c1cnn2c(=O)cc(-c3ccc(C(C)(C)C)cc3)[nH]c12]

The title compound was prepared according to the procedure of Example 13, step 3, using (4-(tert-butyl)phenyl)boronic acid to obtain a white solid (29.0 mg, 41.3% yield). ¹H NMR (400 MHz, DMSO-d₆) δ 11.67 (s, 1H), 8.21 (s, 1H), 7.78 (d, J=7.8 Hz, 2H), 7.58 (d, J=8.0 Hz, 2H), 6.22 (s, 1H), 4.28 (q, J=7.0 Hz, 2H), 1.36-1.30 (m, 12H); LCMS (ESI+) m/z 340.1 (M+H)⁺.

### EXAMPLE 31

### Ethyl 5-(4-isopropoxyphenyl)-7-oxo-4,7-dihydropyrazolo[1,5-a]pyrimidine-3-carboxylate

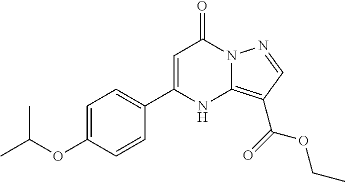 [SMILES: CCOC(=O)c1cnn2c(=O)cc(-c3ccc(OC(C)C)cc3)[nH]c12]

The title compound was prepared according to the procedure of Example 13, step 3, using (4-isopropoxyphenyl)boronic acid to obtain a white solid (34.4 mg, 48.7% yield). ¹H NMR (400 MHz, DMSO-d₆) δ 11.52 (s, 1H), 8.24 (s, 1H), 7.79-7.70 (m, 2H), 7.20-7.05 (m, 2H), 6.22 (s, 1H), 4.82-4.71 (m, 1H), 4.31 (q, J=7.1 Hz, 2H), 1.34 (t, J=7.1 Hz, 3H), 1.30 (d, J=6.0 Hz, 6H). LCMS (ESI+) m/z 342.1 (M+H)⁺.

### EXAMPLE 32

### Ethyl 5-(4-((cyclopropylmethyl)(methyl)amino)phenyl)-7-oxo-4,7-dihydro pyrazolo[1,5-a]pyrimidine-3-carboxylate

The reaction scheme was as follows:

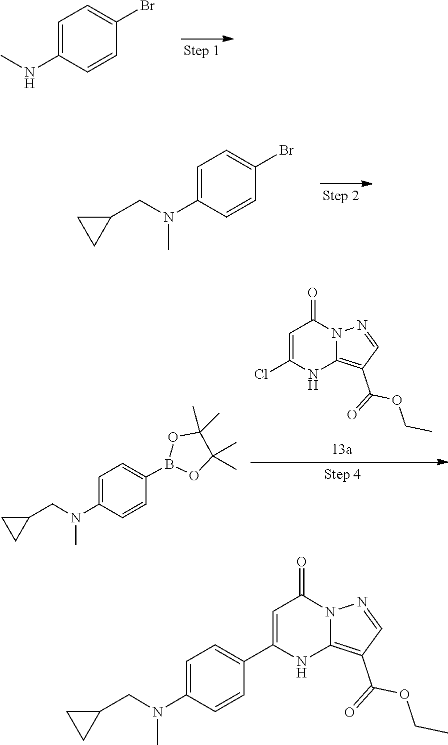

Step 1: 4-Bromo-N-(cyclopropylmethyl)-N-methylaniline

A mixture of N-(4-bromophenyl)-N-methylamine (500 mg, 2.69 mmol), cyclopropane carboxaldehyde (244 mg, 3.49 mmol) and sodium triacetoxyborohydride (1.7 g, 8.06 mmol) in DCE (15 mL) was stirred at 20° C. for 16 h. The reaction mixture was quenched with H₂O (50 mL) and diluted with EtOAc. The organic layer was washed with brine, dried over anhydrous Na₂SO₄, filtered, and concentrated under reduced pressure. The crude residue was purified by silica gel column chromatography (0-5% EtOAc in petroleum ether) to afford 4-bromo-N-(cyclopropylmethyl)-N-methylaniline as an oil (600 mg, 93% yield); LCMS (ESI+) m/z 239.9 (M+H)⁺.

Step 2: N-(Cyclopropylmethyl)-N-methyl-4-(4,4,5,5-tetramethyl-1,3,2-dioxaborolan-2-yl)aniline

The title compound was prepared according to the procedure of Example 13, step 2, using 4-bromo-N-(cyclopropylmethyl)-N-methylaniline (500 mg, 48% yield). LCMS (ESI+) m/z 288.1 (M+H)⁺.

Step 3: Ethyl 5-(4-((cyclopropylmethyl)(methyl)amino)phenyl)-7-oxo-4,7-dihydropyrazolo[1,5-a]pyrimidine-3-carboxylate

The title compound was prepared according to the procedure of Example 13, step 3, using N-(cyclopropylmethyl)-N-methyl-4-(4,4,5,5-tetramethyl-1,3,2-dioxaborolan-2-yl) aniline (19 mg, 16% yield). ¹H NMR (400 MHz, DMSO-d₆) δ11.10 (s, 1H), 8.20 (s, 1H), 7.66 (d, J=8.8 Hz, 2H), 6.89 (d, J=8.8 Hz, 2H), 6.18 (s, 1H), 4.31 (q, J=6.8 Hz, 2H), 3.36 (s, 2H), 3.04 (s, 3H), 1.36 (t, J=6.8 Hz, 3H), 1.02 (s, 1H), 0.48-0.44 (m, 2H), 0.29-0.27 (m, 2H); LCMS (ESI) m/z 367.1 (M+H)⁺.

### EXAMPLE 33

### Ethyl 7-oxo-5-(4-(1-phenylcyclopropyl)phenyl)-4,7-dihydropyrazolo[1,5-a]pyrimidine-3-carboxylate

The reaction scheme was as follows:

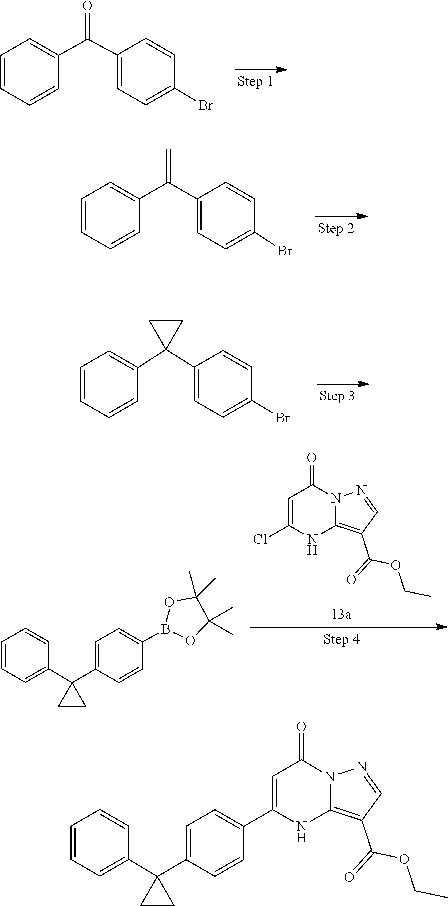 [SMILES: O=C(c1ccccc1)c1ccc(Br)cc1 ; C=C(c1ccccc1)c1ccc(Br)cc1 ; Brc1ccc(C2(c3ccccc3)CC2)cc1 ; CC1(C)OB(c2ccc(C3(c4ccccc4)CC3)cc2)OC1(C)C ; CCOC(=O)c1cnn2c(=O)cc(Cl)[nH]c12 ; CCOC(=O)c1cnn2c(=O)cc(-c3ccc(C4(c5ccccc5)CC4)cc3)[nH]c12]

Step 1: 1-Bromo-4-(1-phenylvinyl)benzene

To a solution of methyltriphenylphosphonium bromide (656 mg, 1.84 mmol) in THF (20 mL) was added n-butyllithium (2.5 M, 0.8 mL, 1.99 mmol) at −78° C., and the reaction mixture was stirred for 30 min at this temperature. 4-Bromobenzophenone (400 mg, 1.53 mmol) was added, and the reaction mixture was stirred for 2 h. The reaction mixture was diluted with H₂O (50 mL) and extracted with EtOAc (30 mL×3). The combined organic layers were washed with brine (50 mL×2), dried over Na₂SO₄, filtered, and concentrated under reduced pressure. The residue was purified by silica gel column chromatography (0%-10% EtOAc in petroleum ether) to give 1-bromo-4-(1-phenylvinyl)benzene as a colorless oil (100 mg, 25% yield). ¹H NMR (400 MHz, CDCl₃) δ 7.48-7.35 (m, 2H), 7.36-7.33 (m, 5H), 7.23-7.21 (m, 2H), 5.48-5.46 (m, 2H).

Step 2: 1-Bromo-4-(1-phenylcyclopropyl)benzene

To a solution of trimethylsulfoxonium iodide (340 mg, 1.54 mmol) in dimethyl sulfoxide (5 mL) was added sodium tert-butoxide (148 mg, 1.54 mmol) at RT. After 30 min, a solution of 1-bromo-4-(1-phenylvinyl)benzene (200 mg, 0.77 mmol) in THF (5 mL) was added. The reaction mixture was stirred at RT for 1 h and heated to 80° C. for 16 h. The reaction mixture was diluted with H₂O (50 mL) and extracted with EtOAc (30 mL×2). The combined organic layers were washed with brine (50 mL), dried over Na₂SO₄, filtered, and concentrated under reduced pressure. The crude residue was purified by prep-TLC (petroleum ether) to give 1-bromo-4-(1-phenylcyclopropyl)benzene as a colorless oil (80 mg, 37% yield). ¹H NMR (400 MHz, CDCl₃) δ 7.39 (d, J=8.8 Hz, 2H), 7.30-7.26 (m, 2H), 7.21-7.19 (m, 3H), 7.10 (d, J=8.4 Hz, 2H), 1.34-1.31 (m, 2H), 1.28-1.25 (m, 2H).

Step 3: 4,4,5,5-Tetramethyl-2-(4-(1-phenylcyclopropyl)phenyl)-1,3,2-dioxaborolane

4,4,5,5-Tetramethyl-2-(4-(1-phenylcyclopropyl)phenyl)-1,3,2-dioxaborolane was prepared according to the procedure of Example 13, step 2, using 1-bromo-4-(1-phenylcyclopropyl)benzene to obtain 4,4,5,5-tetramethyl-2-(4-(1-phenylcyclopropyl)phenyl)-1,3,2-dioxaborolane as a brown solid (80 mg, 85% yield), which was used directly without further purification.

Step 4: Ethyl 7-oxo-5-(4-(1-phenylcyclopropyl)phenyl)-4,7-dihydropyrazolo[1,5-a]pyrimidine-3-carboxylate

The title compound was prepared according to the procedure of Example 13, step 3, using 4,4,5,5-tetramethyl-2-(4-(1-phenylcyclopropyl)phenyl)-1,3,2-dioxaborolane to obtain a white solid (12.1 mg, 9% yield). ¹H NMR (400 MHz, DMSO-d₆) δ 8.07 (s, 1H), 7.91-7.80 (m, 2H), 7.28-7.20 (m, 7H), 6.14 (s, 1H), 4.25-4.20 (m, 2H), 1.35-1.25 (m, 7H); LCMS (ESI) m/z 400 (M+H)⁺.

### EXAMPLE 34

### Ethyl 5-([1,1¹-biphenyl]-4-yl)-7-oxo-4,7-dihydropyrazolo[1,5-a]pyrimidine-3-carboxylate

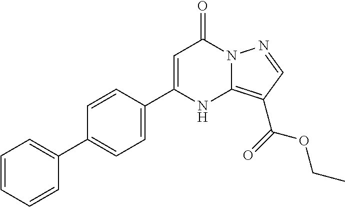 [SMILES: CCOC(=O)c1cnn2c(=O)cc(-c3ccc(-c4ccccc4)cc3)[nH]c12]

The title compound was prepared according to the procedure of Example 1, steps 1 and 2, using phenyl boronic acid to obtain a solid (200 mg, 67% yield over two steps). LCMS (ESI+) m/z 359.9 (M+H)⁺.

### EXAMPLE 35

### 5-([1,1′-Biphenyl]-4-yl)-3-(azetidine-1-carbonyl)pyrazolo[1,5-a]pyrimidin-7(4H)-one

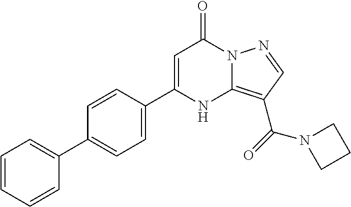 [SMILES: O=C(c1cnn2c(=O)cc(-c3ccc(-c4ccccc4)cc3)[nH]c12)N1CCC1]

The title compound was prepared according to the procedure of Example 8 using ethyl 5-([1,1′-biphenyl]-4-yl)-7-oxo-4,7-dihydropyrazolo[1,5-a]pyrimidine-3-carboxylate from example 34 to obtain a solid (13.5 mg, 11.6% yield over 2 steps). ¹H NMR (400 MHz, DMSO-d₆) δ 11.5 (s, 1H), 8.20 (s, 1H), 7.91 (d, J=8.4 Hz, 4H), 7.78 (d, J=7.2 Hz, 2H), 7.54-7.46 (m, 2H), 7.50-7.44 (m, 1H), 6.40 (s, 1H), 4.52-4.45 (m, 2H), 4.20-4.01 (m, 2H), 2.37-2.33 (m, 2H); LCMS (ESI+) m/z 370.9 (M+H)⁺.

### EXAMPLE 36

### 5-(4-Cyclobutylphenyl)-N,N-dimethyl-7-oxo-4,7-dihydropyrazolo[1,5-a]pyrimidine-3-carboxamide

The reaction scheme was as follows:

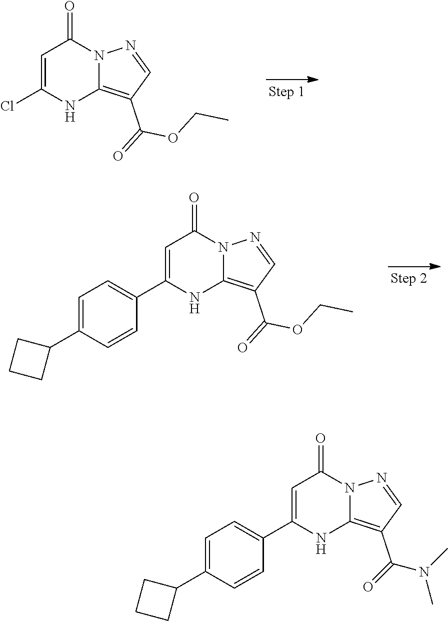 [SMILES: CCOC(=O)c1cnn2c(=O)cc(Cl)[nH]c12 ; CCOC(=O)c1cnn2c(=O)cc(-c3ccc(C4CCC4)cc3)[nH]c12 ; CN(C)C(=O)c1cnn2c(=O)cc(-c3ccc(C4CCC4)cc3)[nH]c12]

Step 1: Ethyl 5-(4-cyclobutylphenyl)-7-oxo-4,7-dihydropyrazolo[1,5-a]pyrimidine-3-carboxylate

Ethyl 5-(4-cyclobutylphenyl)-7-oxo-4,7-dihydropyrazolo[1,5-a]pyrimidine-3-carboxylate was prepared according to the procedure of Example 13, step 3, using (4-cyclobutylphenyl)boronic acid to obtain a white solid (350 mg, 59% yield). ¹H NMR (400 MHz, CD₃OD) δ 8.25 (s, 1H), 7.74 (d, J=8.4 Hz, 2H), 7.46 (d, J=8.4 Hz, 2H), 6.26 (s, 1H), 4.40 (q, J=7.2 Hz, 2H), 3.72-3.63 (m, 1H), 2.49-2.36 (m, 2H), 2.29-2.08 (m, 3H), 1.99-1.87 (m, 1H), 1.41 (t, J=7.2 Hz, 3H); LCMS (ESI+) m/z 337.9 (M+H)⁺.

Step 2: 5-(4-Cyclobutylphenyl)-N,N-dimethyl-7-oxo-4,7-dihydropyrazolo[1,5-a]pyrimidine-3-carboxamide

The title compound was prepared according to the procedure of Example 3 using ethyl 5-(4-cyclobutylphenyl)-7-oxo-4,7-dihydropyrazolo[1,5-a]pyrimidine-3-carboxylate to obtain a white solid (17.1 mg, 6.5% yield). 1H NMR (400 MHz, CDCl₃) δ 11.24 (s, 1H), 8.13 (s, 1H), 7.66 (d, J=8.0 Hz, 2H), 7.39 (d, J=8.0 Hz, 2H), 6.28 (s, 1H), 3.68-3.62 (m, 1H), 3.43 (s, 3H), 3.18 (s, 3H), 2.48-2.35 (m, 2H), 2.24-2.02 (m, 3H), 1.97-1.84 (m, 1H); LCMS (ESI+) m/z 336.9 (M+H)⁺.

### EXAMPLE 37

### (R)-1-(5-(4-Cyclohexylphenyl)-7-oxo-4,7-dihydropyrazolo[1,5-a]pyrimidine-3-carbonyl)pyrrolidine-3-carbonitrile

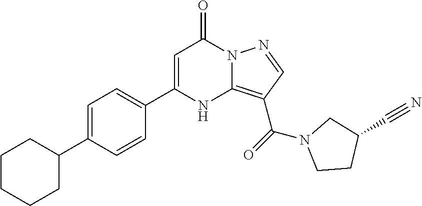 [SMILES: N#C[C@@H]1CCN(C(=O)c2cnn3c(=O)cc(-c4ccc(C5CCCCC5)cc4)[nH]c23)C1]

To a mixture of 5-(4-cyclohexylphenyl)-7-oxo-4,7-dihydropyrazolo[1,5-a]pyrimidine-3-carboxylic acid (200.0 mg, 0.59 mmol), HATU (338.1 mg, 0.89 mmol) in DMF (3 mL) was added DIPEA (0.52 mL, 2.96 mmol), and the mixture was stirred at 20° C. for 30 min. (3R)-Pyrrolidine-3-carbonitrile hydrochloride (117.9 mg, 0.89 mmol) was added into mixture and the solution was stirred at 20° C. for further 2 hours. The resulting solution was purified by pre-HPLC (acetonitrile 55-85/0.225% HCOOH in water) to afford the title compound (120.0 mg, 48%) as a white solid. ¹H NMR (400 MHz, CDCl₃): δ 11.08 (s, 1H), 8.10 (s, 1H), 7.64 (d, J=8.4 Hz, 2H), 7.39 (d, J=8.4 Hz, 2H), 6.30 (s, 1H), 4.31-3.76 (m, 4H), 3.49-3.18 (m, 1H), 2.67-2.49 (m, 2H), 2.45-2.32 (m, 1H), 1.95-1.84 (m, 4H), 1.83-1.75 (m, 1H), 1.53-1.35 (m, 4H), 1.34-1.21 (m, 1H); LCMS (ESI): m/z 416.1 (M+H)⁺.

### EXAMPLE 38

### 5-(4-Cyclohexylphenyl)-3-(3-(difluoromethyl)azetidine-1-carbonyl)pyrazolo[1,5-a]pyrimidin-7(4H)-one

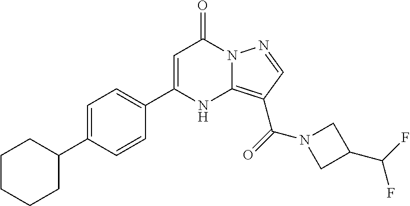 [SMILES: O=C(c1cnn2c(=O)cc(-c3ccc(C4CCCCC4)cc3)[nH]c12)N1CC(C(F)F)C1]

The title compound (25.2 mg, 10%) was furnished as a white solid, which was prepared from 5-(4-cyclohexylphenyl)-7-oxo-4,7-dihydropyrazolo[1,5-a]pyrimidine-3-carboxylic acid (200 mg, 0.59 mmol) and 3-(difluoromethyl)azetidine hydrochloride (171 mg, 1.19 mmol) following the procedure outlined for Example 5; ¹H NMR (400 MHz, DMSO-d₆): δ 11.30 (s, 1H), 8.11 (s, 1H), 7.88-7.85 (m, 2H), 7.38-7.35 (m, 2H), 6.55-6.15 (m, 2H), 4.72-4.36 (m, 2H), 4.23-3.89 (m, 2H), 3.21-3.18 (m, 1H), 2.59-2.56 (m, 1H), 1.83-1.69 (m, 5H), 1.47-1.26 (m, 5H); LCMS (ESI): m/z 427.1 (M+H)⁺.

### EXAMPLE 39

### 5-(4-Cyclohexylphenyl)-3-(3-(trifluoromethyl)azetidine-1-carbonyl)pyrazolo[1,5-a]pyrimidin-7(4H)-one

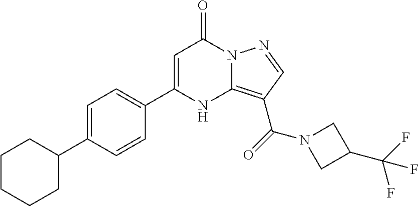 [SMILES: O=C(c1cnn2c(=O)cc(-c3ccc(C4CCCCC4)cc3)[nH]c12)N1CC(C(F)(F)F)C1]

The title compound (95.2 mg, 59%) was furnished as a white solid, which was prepared from 5-(4-cyclohexylphenyl)-7-oxo-4,7-dihydropyrazolo[1,5-a]pyrimidine-3-carboxylic acid (120 mg, 0.36 mmol) and 3-(trifluoromethyl)azetidine hydrochloride (86.2 mg, 0.53 mmol) following the procedure outlined for Example 5; ¹H NMR (400 MHz, DMSO-d₆): δ 11.29 (s, 1H), 8.28 (s, 1H), 7.73 (d, J=8.4 Hz, 2H), 7.45 (d, J=8.4 Hz, 2H), 6.32 (s, 1H), 4.80-4.47 (m, 2H), 4.41-3.95 (m, 2H), 3.86-3.66 (m, 1H), 2.63-2.58 (m, 1H), 1.84-1.76 (m, 4H), 1.72 (m, 1H), 1.53-1.32 (m, 4H), 1.31-1.21 (m, 1H); LCMS (ESI): m/z 445.2 (M+H)⁺.

### EXAMPLE 40

### 5-(4-Cyclohexylphenyl)-3-(3-fluoro-3-methylazetidine-1-carbonyl)pyrazolo[1,5-a]pyrimidin-7(4H)-one

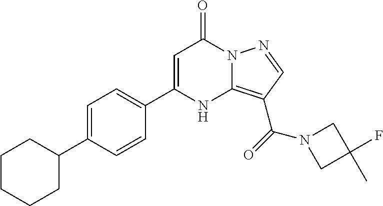 [SMILES: CC1(F)CN(C(=O)c2cnn3c(=O)cc(-c4ccc(C5CCCCC5)cc4)[nH]c23)C1]

The title compound (37.4 mg, 15%) was furnished as a white solid, which was prepared from 5-(4-cyclohexylphenyl)-7-oxo-4,7-dihydropyrazolo[1,5-a]pyrimidine-3-carboxylic acid (200 mg, 0.59 mmol) and 3-fluoro-3-methyl-azetidine hydrochloride (149 mg, 1.19 mmol) following the procedure outlined for Example 5. ¹H NMR (400 MHz, DMSO-d₆): δ 11.31 (s, 1H), 8.20 (s, 1H), 7.75 (d, J=6.8 Hz, 2H), 7.45 (d, J=6.8 Hz, 2H), 6.31 (s, 1H), 4.60-4.50 (m, 2H), 4.30-4.15 (m, 2H), 2.68-2.57 (m, 1H), 1.84-1.61 (m, 8H), 1.50-1.24 (m, 5H); LCMS (ESI): m/z 409.1 (M+H)⁺.

### EXAMPLE 41

### 5-(4-Cyclohexylphenyl)-3-(3-(fluoromethyl)azetidine-1-carbonyl)pyrazolo[1,5-a]pyrimidin-7(4H)-one

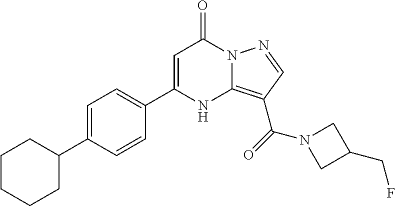 [SMILES: O=C(c1cnn2c(=O)cc(-c3ccc(C4CCCCC4)cc3)[nH]c12)N1CC(CF)C1]

The title compound (74.3 mg, 49%) was furnished as a white solid, which was prepared from 5-(4-cyclohexylphenyl)-7-oxo-4,7-dihydropyrazolo[1,5-a]pyrimidine-3-carboxylic acid (120 mg, 0.36 mmol) and 3-(fluoromethyl)azetidine hydrochloride (67 mg, 0.53 mmol) following the procedure outlined for Example 5. ¹H NMR (400 MHz, CDCl₃): δ 10.85 (s, 1H), 7.98 (s, 1H), 7.64 (d, J=8.4 Hz, 2H), 7.39 (d, J=8.4 Hz, 2H), 6.28 (s, 1H), 4.76-4.52 (m, 3H), 4.47-4.27 (m, 2H), 4.14-4.06 (m, 1H), 3.24-3.11 (m, 1H), 2.63-2.55 (m, 1H), 1.94-1.84 (m, 4H), 1.82-1.75 (m, 1H), 1.48-1.39 (m, 4H), 1.35-1.23 (m, 1H); LCMS (ESI): m/z 409.2 (M+H)⁺.

### EXAMPLE 42

5-(4-Cyclohexylphenyl)-3-(3,3-difluoropyrrolidine-1-carbonyl)pyrazolo[1,5-a]pyrimidin-7(4H)-one

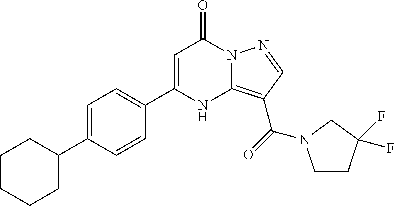 [SMILES: O=C(c1cnn2c(=O)cc(-c3ccc(C4CCCCC4)cc3)[nH]c12)N1CCC(F)(F)C1]

The title compound (105.7 mg, 84%) was furnished as a white solid, which was prepared from 5-(4-cyclohexylphenyl)-7-oxo-4,7-dihydropyrazolo[1,5-a]pyrimidine-3-carboxylic acid (100 mg, 0.300 mmol) and 3,3-difluoropyrrolidine hydrochloride (64.4 mg, 0.45 mmol) following the procedure outlined for Example 5. LCMS (ESI): m/z 427.2 (M+H)⁺.

### EXAMPLE 43

### (R)-5-(4-cyclohexylphenyl)-3-(2-(fluoromethyl)azetidine-1-carbonyl)pyrazolo[1,5-a]pyrimidin-7(4H)-one

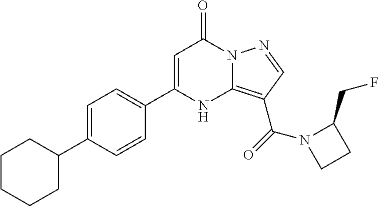 [SMILES: O=C(c1cnn2c(=O)cc(-c3ccc(C4CCCCC4)cc3)[nH]c12)N1CC[C@@H]1CF]

The title compound (56.4 mg, 47%) was furnished as a white solid, which was prepared from 5-(4-cyclohexylphenyl)-7-oxo-4,7-dihydropyrazolo[1,5-a]pyrimidine-3-carboxylic acid (100 mg, 0.300 mmol) and (2R)-2-(fluoromethyl)azetidine hydrochloride (56.3 mg, 0.45 mmol) following the procedure outlined for Example 5; LCMS (ESI): m/z 409.1 (M+H)⁺.

### EXAMPLE 44

### 1-(5-(4-Cyclohexylphenyl)-7-oxo-4,7-dihydropyrazolo[1,5-a]pyrimidine-3-carbonypazetidine-3-carbonitrile

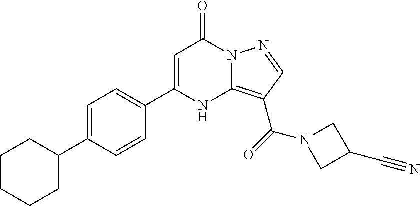 [SMILES: N#CC1CN(C(=O)c2cnn3c(=O)cc(-c4ccc(C5CCCCC5)cc4)[nH]c23)C1]

The title compound (320 mg, 45%) was furnished as a white solid, which was prepared from 5-(4-cyclohexylphenyl)-7-oxo-4,7-dihydropyrazolo[1,5-a]pyrimidine-3-c arb oxylic acid (600 mg, 1.78 mmol) and Azetidine-3-carbonitrile hydrochloride (0.38 g, 3.20 mmol) following the procedure outlined for Example 5. ¹H NMR (400 MHz, DMSO-d₆): δ 11.34 (s, 1H), 8.21 (s, 1H), 7.73 (d, J=8.0 Hz, 2H), 7.45 (d, J=8.0 Hz, 2H), 6.31 (s, 1H), 4.80-4.14 (m, 4H), 3.99-3.90 (m, 1H), 2.64-2.58 (m, 1H), 1.82-1.78 (m, 4H), 1.73-1.70 (m, 1H), 1.47-1.36 (m, 4H), 1.30-1.22 (m, 1H); LCMS (ESI): m/z 402.2 (M+H)⁺.

### EXAMPLES 45 & 46

### 5-(4-cyclohexylphenyl)-3-(3-(fluoromethyl)-2-methylazetidine-1-carbonyl)pyrazolo[1,5-a]pyrimidin-7(4H)-one

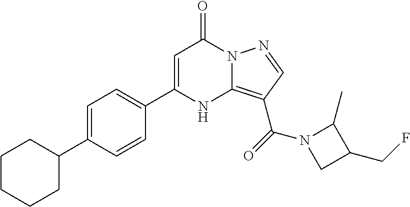 [SMILES: CC1C(CF)CN1C(=O)c1cnn2c(=O)cc(-c3ccc(C4CCCCC4)cc3)[nH]c12]

### 5-(4-Cyclohexylphenyl)-3-((2R,3R)-3-(fluoromethyl)-2-methylazetidine-1-carbonyl) pyrazolo[1,5-a]pyrimidin-7(4H)-one

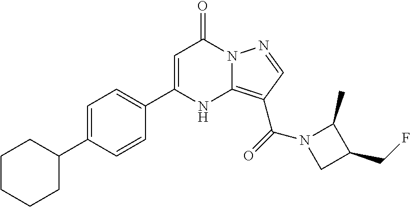 [SMILES: C[C@H]1[C@@H](CF)CN1C(=O)c1cnn2c(=O)cc(-c3ccc(C4CCCCC4)cc3)[nH]c12]

### 5-(4-cyclohexylphenyl)-3-((2S,3S)-3-(fluoro methyl)-2-methylazetidine-1-carbonyl)pyrazolo[1,5-a]pyrimidin-7(4H)-one

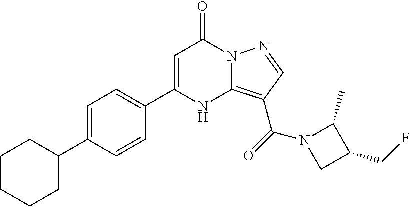 [SMILES: C[C@@H]1[C@H](CF)CN1C(=O)c1cnn2c(=O)cc(-c3ccc(C4CCCCC4)cc3)[nH]c12]

Step 1: 5-(4-Cyclohexylphenyl)-3-(cis-3-(fluoromethyl)-2-methylazetidine-1-carbonyl)pyrazolo[1,5-a]pyrimidin-7(4H)-one

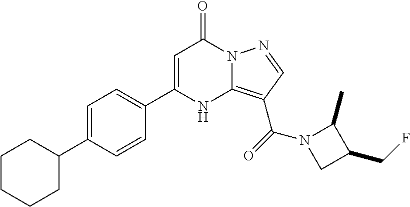 [SMILES: C[C@H]1[C@@H](CF)CN1C(=O)c1cnn2c(=O)cc(-c3ccc(C4CCCCC4)cc3)[nH]c12]

To the solution of 5-(4-cyclohexylphenyl)-7-oxo-4H-pyrazolo[1,5-a]pyrimidine-3-carboxylic acid (250 mg, 0.74 mmol) and HATU (422 mg, 1.11 mmol) in DMF (2.5 mL) was added N, N-diisopropylethylamine (0.39 mL, 2.22 mmol). The reaction mixture was stirred at 25° C. for 10 min. Then cis-3-(fluoromethyl)-2-methyl-azetidine 2,2,2-trifluoroacetate (241 mg, 1.11 mmol) was added and the solution was stirred at 25° C. for another 2 h. Brine (30 mL) was added to the reaction. The resulting precipitate was filtered. The filter cake was washed with ethyl acetate (10 mL) and further purified by prep-TLC (2% methanol in dichloromethane) to afford the title compound (200 mg, 63% yield) as a white solid. ¹H NMR (400 MHz, DMSO-d₆): δ 11.27 (br s, 1H), 8.18 (s, 1H), 7.77-7.75 (m, 2H), 7.44-7.42 (m, 2H), 6.32 (s, 1H), 4.88-4.62 (m, 3H), 4.45-4.28 (m, 2H), 3.18-3.05 (m, 1H), 2.63-2.60 (m, 1H), 1.82-1.80 (m, 4H), 1.73-1.70 (m, 1H), 1.46-1.35 (m, 7H), 1.31-1.22 (m, 1H); LCMS (ESI): m/z 423.1 (M+H)⁺.

Step 2: 5-(4-Cyclohexylphenyl)-3-((2R,3R)-3-(fluoromethyl) 2-methylazetidine-1-carbonyl)pyrazolo[1,5-a]pyrimidin-7(4H)-one 5-(4-cyclohexylphenyl)-3-((2S,3S)-3-(fluoromethyl)-2-methylazetidine-1-carbonyl)pyrazolo[1,5-a]pyrimidin-7(4H)-one

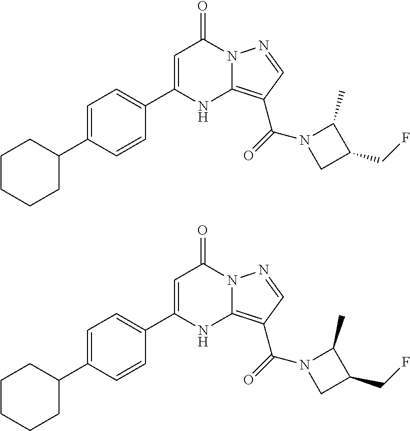 [SMILES: C[C@@H]1[C@H](CF)CN1C(=O)c1cnn2c(=O)cc(-c3ccc(C4CCCCC4)cc3)[nH]c12 ; C[C@H]1[C@@H](CF)CN1C(=O)c1cnn2c(=O)cc(-c3ccc(C4CCCCC4)cc3)[nH]c12]

5-(4-Cyclohexylphenyl)-3-[cis-3-(fluoromethyl)-2-methyl-azetidine-1-carbonyl]-4H-pyrazolo[1,5-a]pyrimidin-7-one (200 mg, 0.47 mmol) was resolved by preparative SFC (Column: DAICEL CHIRALCEL OJ (250 mm*30 mm,10 um); Condition: 0.1% NH₃H₂O/EtOH) to give 5-(4-cyclohexylphenyl)-3-[(2R,3R)-3-(fluoromethyl)-2-methyl-azetidine-1-carbonyl]-4H-pyrazolo[1,5-a]pyrimidin-7-one (72 mg, 36% yield) as a white solid (Peak 2 on SFC) and 5-(4-cyclohexylphenyl)-3-[(2S,3S)-3-(fluoromethyl)-2-methyl-azetidine-1-carbonyl]-4H-pyrazolo[1,5-a]pyrimidin-7-one (90 mg, 76% de) (Peak 3 on SFC) as a white solid.

5-(4-Cyclohexylphenyl)-3-[(2R,3R)-3-(fluoromethyl)-2-methyl-azetidine-1-carbonyl]-4H-pyrazolo[1,5-a]pyrimidin-7-one. ¹H NMR (400 MHz, DMSO-d₆): δ 11.29 (br s, 1H), 8.17 (s, 1H), 7.76 (d, J=8.0 Hz, 2H), 7.43 (d, J=8.0 Hz, 2H), 6.29 (s, 1H), 4.88-4.62 (m, 3H), 4.45-4.28 (m, 2H), 3.18-3.05 (m, 1H), 2.63-2.60 (m, 1H), 1.82-1.80 (m, 4H), 1.73-1.70 (m, 1H), 1.47-1.35 (m, 7H), 1.31-1.22 (m, 1H); LCMS (ESI): m/z 423.1 (M+H)⁺.

5-(4-Cyclohexylphenyl)-3-[(2S,3S)-3-(fluoromethyl)-2-methyl-azetidine-1-carbonyl]-4H-pyrazolo[1,5-a]pyrimidin-7-one (90 mg, 76% de) was further purified by preparative SFC (Column: YMC CHIRAL Amylose-C(250 mm*30 mm, 10 um; Condition: 0.1% NH₃H₂O/IPA) to give the title compound (52 mg, 26% yield) (Peak 1 on SFC) as a white solid. ¹H NMR (400 MHz, DMSO-d₆): δ 11.28 (br s, 1H), 8.19 (s, 1H), 7.74 (d, J=8.0 Hz, 2H), 7.45 (d, J=8.0 Hz, 2H), 6.31 (s, 1H), 4.88-4.61 (m, 3H), 4.54-4.10 (m, 2H), 3.15-3.12 (m, 1H), 2.63-2.58 (m, 1H), 1.82-1.79 (m, 4H), 1.72-1.70 (m, 1H), 1.47-1.36 (m, 7H), 1.31-1.23 (m, 1H); LCMS (ESI): m/z 423.1 (M+H)⁺.

### ABBREVIATIONS

AcOH Acetic acid
Cs₂CO₃ Cesium carbonate
DCE 1,2-Dichloroethane
DCM Dichloromethane
DIAD Diisopropyl azodicarboxylate
DMF N,N-Dimethylformamide
DMSO Dimethyl sulfoxide
EtOAc Ethyl acetate
EtOH Ethanol
HATU 1-[Bis(dimethylamino)methylene]-1H-1,2,3-triazolo[4,5-b]pyridinium 3-oxid hexafluorophosphate
HCl Hydrochloric acid
H₂O Water
KOAc Potassium acetate
MeOH Methanol
NaH Sodium hydride
NaHCO₃ Sodium bicarbonate
Na₂CO₃ Sodium carbonate
Na₂SO₃ Sodium sulfite
Na₂SO₄Sodium sulfate
NBS N-bromosuccinimide
p-TsOH.H₂O p-Toluenesulfonic acid monohydrate
Pd(dppf)Cl₂ 1,1′-Bis(diphenylphosphino)ferrocene palladium(II) dichloride
PPh₃ Triphenylphosphine
THF Tetrahydrofuran

### EXAMPLE 47

Purified His-tagged TEAD protein (YAP Binding Domain, amino acids 217-447) is pre-incubated with Europium labelled anti-His antibody tracer (Perkin Elmer Cat #AD0110). Small molecule Inhibitors are then incubated with the TEAD-Eu protein complex for 30 minutes to allow for binding to TEAD protein. Biotinylated YAP peptide (AA's 50-100) that has been pre-incubated with streptavidin x1665 acceptor (CIS-Bio Cat #610SAXAC) is added to the compound-TEAD mix. The TEAD-YAP-inhibitor mixture is then incubated for 60 minutes at room temperature. All reactions are carried out in a polystyrene plate. After 60 minutes, the plate is read on a plate reader using TR-FRET mode with wavelengths of 665 nm/615 nm. If YAP binds to TEAD as expected, a TR-FRET signal results from the proximity of YAP and TEAD after binding. If an inhibitor such as peptide 17 (Selleckchem Cat #S8164) interferes with YAP-TEAD binding, the disruption of the YAP:TEAD interaction results in a decrease in TR-FRET signal. The potency of compounds as YAP:TEAD protein-protein interaction (PPi) inhibitors is determined by an IC₅₀ or EC₅₀ value generated using a non-linear four parameter curve fit. The extent to which representative examples of the disclosed compounds are able to inhibit interaction between TEAD2 or TEAD3 and YAP truncated from amino acids 50-100 as measured by Homogeneous Time Resolved Fluorescence (HTRF) to generate EC 50 data is provided in Table 3 below.

### Biological Data

TABLE 3

| | | TEAD2 | TEAD3 |
|---|---|---|---|
| | | YAP50-100 | YAP50-100 |
| | | HTRF | HTRF |
| Example | | WuXi | WuXi |
| No. | Structure | EC₅₀ (μM) | EC₅₀ (μM) |
| 1 | | 0.263 | 0.041 |
| 2 | | 0.120 | 0.019 |
| 3 | 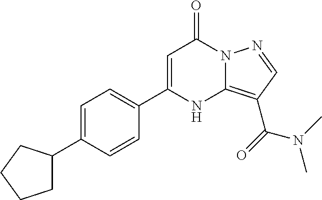 [SMILES: CN(C)C(=O)c1cnn2c(=O)cc(-c3ccc(C4CCCC4)cc3)[nH]c12] | 0.342 | 0.016 |
| 4 | 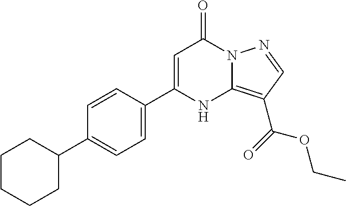 [SMILES: CCOC(=O)c1cnn2c(=O)cc(-c3ccc(C4CCCCC4)cc3)[nH]c12] | 0.066 | 0.019 |
| 5 | 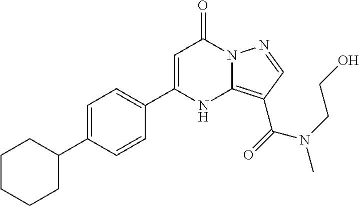 [SMILES: CN(CCO)C(=O)c1cnn2c(=O)cc(-c3ccc(C4CCCCC4)cc3)[nH]c12] | 1.096 | 0.567 |
| 6 | 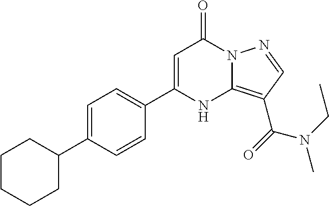 [SMILES: CCN(C)C(=O)c1cnn2c(=O)cc(-c3ccc(C4CCCCC4)cc3)[nH]c12] | 0.067 | 0.018 |
| 7 | 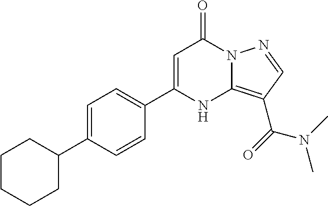 [SMILES: CN(C)C(=O)c1cnn2c(=O)cc(-c3ccc(C4CCCCC4)cc3)[nH]c12] | 0.087 | 0.018 |
| 8 | 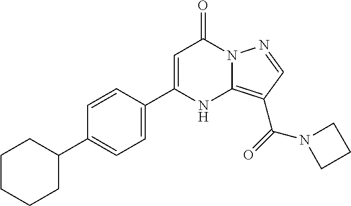 [SMILES: O=C(c1cnn2c(=O)cc(-c3ccc(C4CCCCC4)cc3)[nH]c12)N1CCC1] | 0.038 | 0.023 |
| 9 | 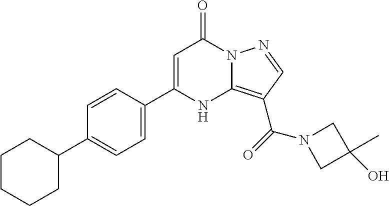 [SMILES: CC1(O)CN(C(=O)c2cnn3c(=O)cc(-c4ccc(C5CCCCC5)cc4)[nH]c23)C1] | 0.333 | 1.300 |
| 10 | 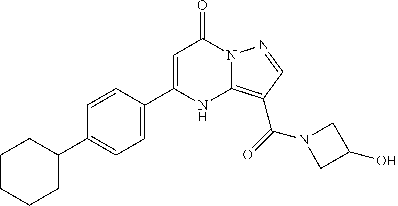 [SMILES: O=C(c1cnn2c(=O)cc(-c3ccc(C4CCCCC4)cc3)[nH]c12)N1CC(O)C1] | 0.548 | 0.072 |
| 11 | 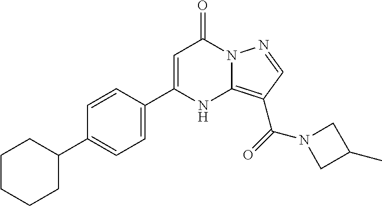 [SMILES: CC1CN(C(=O)c2cnn3c(=O)cc(-c4ccc(C5CCCCC5)cc4)[nH]c23)C1] | 0.011 | 0.032 |
| 12 | 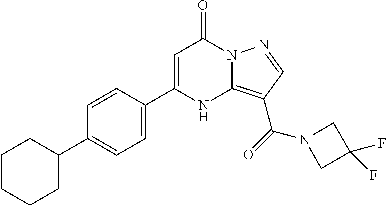 [SMILES: O=C(c1cnn2c(=O)cc(-c3ccc(C4CCCCC4)cc3)[nH]c12)N1CC(F)(F)C1] | 0.068 | 0.056 |
| 13 | 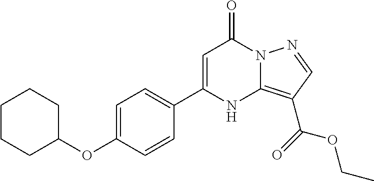 [SMILES: CCOC(=O)c1cnn2c(=O)cc(-c3ccc(OC4CCCCC4)cc3)[nH]c12] | 0.793 | 0.066 |
| 14 | 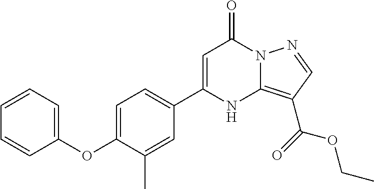 [SMILES: CCOC(=O)c1cnn2c(=O)cc(-c3ccc(Oc4ccccc4)c(C)c3)[nH]c12] | 0.747 | 0.666 |
| 15 | 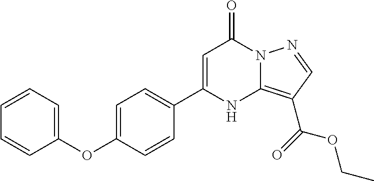 [SMILES: CCOC(=O)c1cnn2c(=O)cc(-c3ccc(Oc4ccccc4)cc3)[nH]c12] | 0.836 | 0.532 |
| 16 | 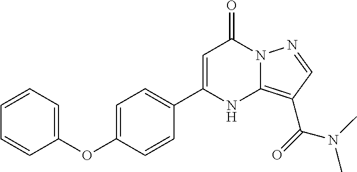 [SMILES: CN(C)C(=O)c1cnn2c(=O)cc(-c3ccc(Oc4ccccc4)cc3)[nH]c12] | 0.501 | 1.113 |
| 17 | 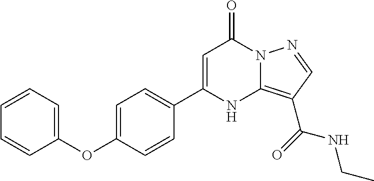 [SMILES: CCNC(=O)c1cnn2c(=O)cc(-c3ccc(Oc4ccccc4)cc3)[nH]c12] | 0.055 | 4.997 |
| 18 | 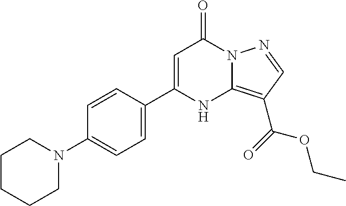 [SMILES: CCOC(=O)c1cnn2c(=O)cc(-c3ccc(N4CCCCC4)cc3)[nH]c12] | 7.35 | 0.239 |
| 19 | 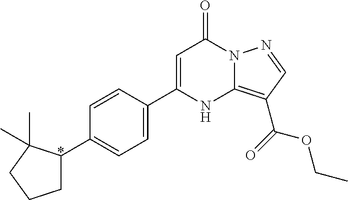 [SMILES: CCOC(=O)c1cnn2c(=O)cc(-c3ccc(C4CCCC4(C)C)cc3)[nH]c12] | 0.270 | 0.037 |
| 20 | 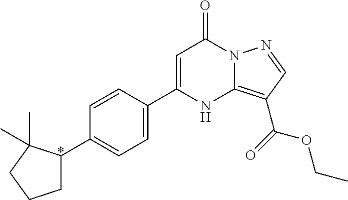 [SMILES: CCOC(=O)c1cnn2c(=O)cc(-c3ccc(C4CCCC4(C)C)cc3)[nH]c12] | 0.187 | 0.060 |
| 21 | 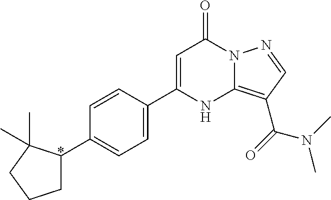 | 0.220 | 0.021 |
| 22 | 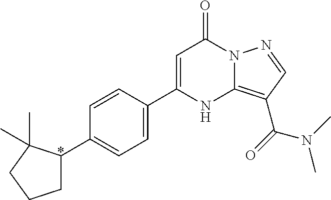 | 0.378 | 0.042 |
| 23 | 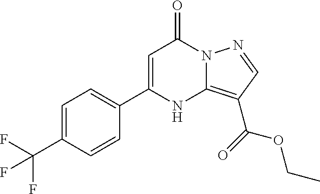 [SMILES: CCOC(=O)c1cnn2c(=O)cc(-c3ccc(C(F)(F)F)cc3)[nH]c12] | 1.777 | 0.834 |
| 24 | 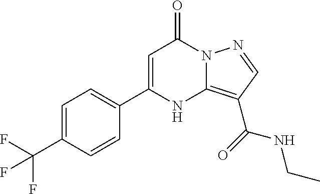 [SMILES: CCNC(=O)c1cnn2c(=O)cc(-c3ccc(C(F)(F)F)cc3)[nH]c12] | 0.080 | 4.380 |
| 25 | 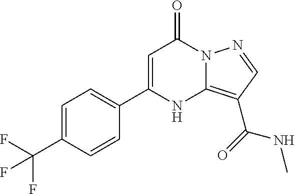 [SMILES: CNC(=O)c1cnn2c(=O)cc(-c3ccc(C(F)(F)F)cc3)[nH]c12] | 0.212 | 10.352 |
| 26 | 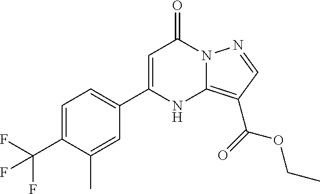 [SMILES: CCOC(=O)c1cnn2c(=O)cc(-c3ccc(C(F)(F)F)c(C)c3)[nH]c12] | 0.956 | 1.411 |
| 27 | 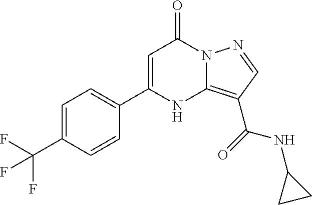 [SMILES: O=C(NC1CC1)c1cnn2c(=O)cc(-c3ccc(C(F)(F)F)cc3)[nH]c12] | 0.211 | 3.514 |
| 28 | 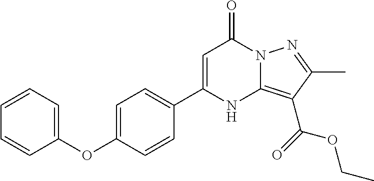 [SMILES: CCOC(=O)c1c(C)nn2c(=O)cc(-c3ccc(Oc4ccccc4)cc3)[nH]c12] | 3.570 | 0.393 |
| 29 | 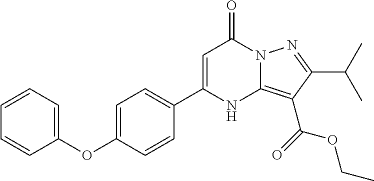 [SMILES: CCOC(=O)c1c(C(C)C)nn2c(=O)cc(-c3ccc(Oc4ccccc4)cc3)[nH]c12] | 0.249 | 0.124 |
| 30 | 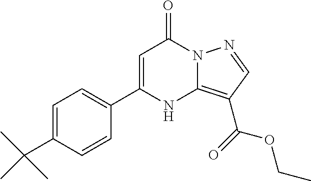 [SMILES: CCOC(=O)c1cnn2c(=O)cc(-c3ccc(C(C)(C)C)cc3)[nH]c12] | 1.622 | 0.095 |
| 31 | 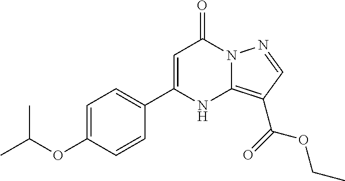 [SMILES: CCOC(=O)c1cnn2c(=O)cc(-c3ccc(OC(C)C)cc3)[nH]c12] | 4.045 | 0.944 |
| 32 | 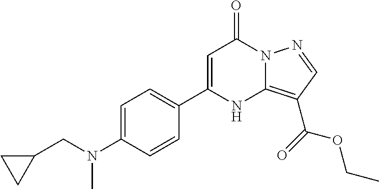 [SMILES: CCOC(=O)c1cnn2c(=O)cc(-c3ccc(N(C)CC4CC4)cc3)[nH]c12] | 3.695 | 0.175 |
| 33 | 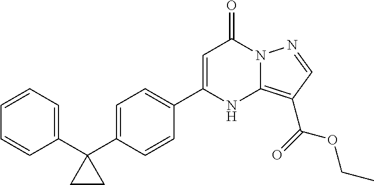 [SMILES: CCOC(=O)c1cnn2c(=O)cc(-c3ccc(C4(c5ccccc5)CC4)cc3)[nH]c12] | 2.191 | 0.765 |
| 34 | 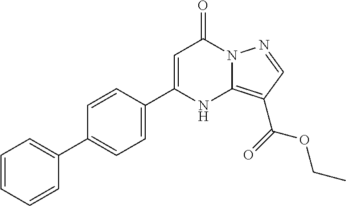 [SMILES: CCOC(=O)c1cnn2c(=O)cc(-c3ccc(-c4ccccc4)cc3)[nH]c12] | 1.028 | 0.041 |
| 35 | 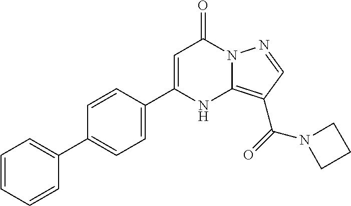 [SMILES: O=C(c1cnn2c(=O)cc(-c3ccc(-c4ccccc4)cc3)[nH]c12)N1CCC1] | 1.631 | 0.095 |
| 36 | 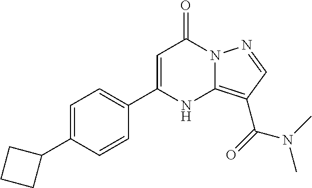 [SMILES: CN(C)C(=O)c1cnn2c(=O)cc(-c3ccc(C4CCC4)cc3)[nH]c12] | 1.871 | 0.028 |
| 37 | 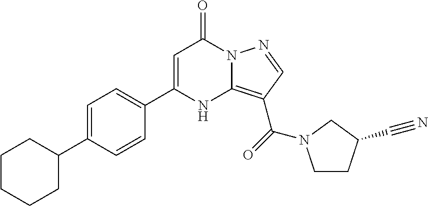 [SMILES: N#C[C@@H]1CCN(C(=O)c2cnn3c(=O)cc(-c4ccc(C5CCCCC5)cc4)[nH]c23)C1] | 0.00879 | 0.0522 |
| 38 | 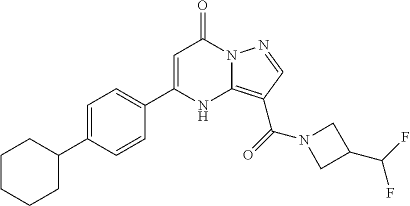 [SMILES: O=C(c1cnn2c(=O)cc(-c3ccc(C4CCCCC4)cc3)[nH]c12)N1CC(C(F)F)C1] | 0.012 | 0.027 |
| 39 | 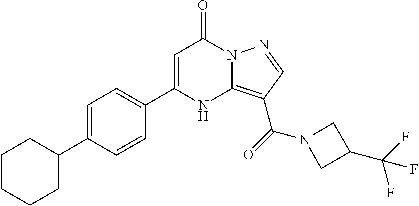 [SMILES: O=C(c1cnn2c(=O)cc(-c3ccc(C4CCCCC4)cc3)[nH]c12)N1CC(C(F)(F)F)C1] | 0.295 | 0.405 |
| 40 | 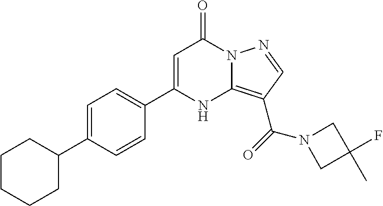 [SMILES: CC1(F)CN(C(=O)c2cnn3c(=O)cc(-c4ccc(C5CCCCC5)cc4)[nH]c23)C1] | 0.0125 | 0.0185 |
| 41 | 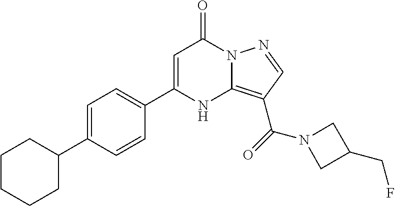 [SMILES: O=C(c1cnn2c(=O)cc(-c3ccc(C4CCCCC4)cc3)[nH]c12)N1CC(CF)C1] | 0.0045 | 0.01 |
| 42 | 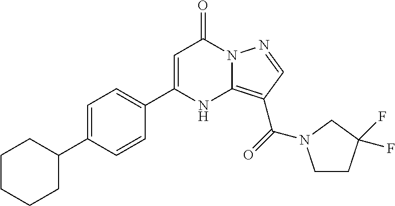 [SMILES: O=C(c1cnn2c(=O)cc(-c3ccc(C4CCCCC4)cc3)[nH]c12)N1CCC(F)(F)C1] | 0.0145 | 0.0395 |
| 43 | 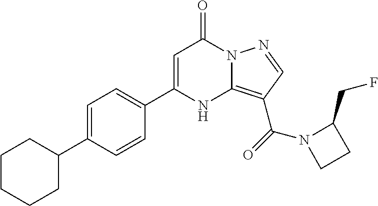 [SMILES: O=C(c1cnn2c(=O)cc(-c3ccc(C4CCCCC4)cc3)[nH]c12)N1CC[C@@H]1CF] | 0.0225 | 0.028 |
| 44 | 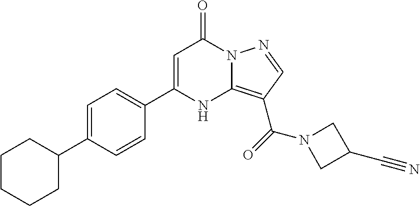 [SMILES: N#CC1CN(C(=O)c2cnn3c(=O)cc(-c4ccc(C5CCCCC5)cc4)[nH]c23)C1] | 0.00585 | 0.013 |
| 45 | 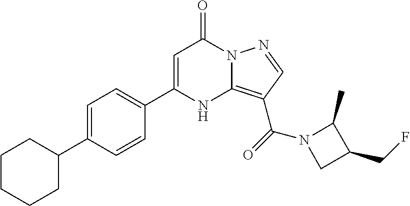 [SMILES: C[C@H]1[C@@H](CF)CN1C(=O)c1cnn2c(=O)cc(-c3ccc(C4CCCCC4)cc3)[nH]c12] | 0.008 | 0.043 |
| 46 | 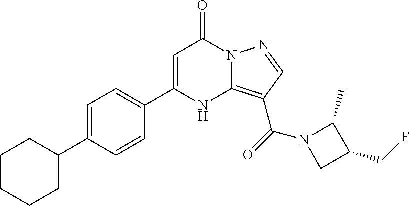 [SMILES: C[C@@H]1[C@H](CF)CN1C(=O)c1cnn2c(=O)cc(-c3ccc(C4CCCCC4)cc3)[nH]c12] | 0.755 | 3.1 |

1-35. (canceled)

36. A compound of formula (I): 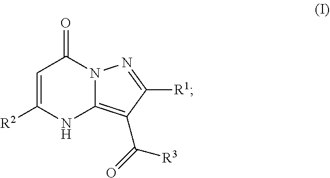
wherein:
R¹ is selected from the group consisting of hydrogen, halogen, C₁₋₁₀alkyl, and C₁₋₁₀haloalkyl;
R² is 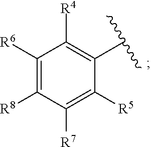
R³ is OR⁹ or NR¹⁰R¹¹, wherein when R² is C₅₋₁₀heteroaryl and R³ is NR¹⁰R¹¹, then each of R¹⁰ and R¹¹ is not hydrogen;
R⁴, R⁵, R⁶, and R⁷ are each independently selected from the group consisting of hydrogen, halogen, C₁₋₁₀alkyl, C₁₋₁₀haloalkyl, O—C₁₋₁₀alkyl, and NR^{a}R^{b};
R⁸ is selected from the group consisting of unsubstituted or substituted C₁₋₁₀alkyl, unsubstituted or substituted C₁₋₁₀haloalkyl, unsubstituted or substituted O—C₁₋₁₀haloalkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, unsubstituted or substituted C₅₋₁₀heteroaryl; and unsubstituted or substituted NR^{c}R^{d}; wherein each R⁸ is optionally substituted with one to five R^{e} groups;
R⁹ is selected from the group consisting of unsubstituted or substituted C₁₋₁₀alkyl, unsubstituted or substituted C₃₋₈cycloalkyl, unsubstituted or substituted C₆₋₁₀aryl, and unsubstituted or substituted C₅₋₁₀heteroaryl; wherein each R⁹ is optionally substituted with one to five R^{e} groups;
R¹⁰ and R¹¹ are each independently selected from the group consisting of hydrogen, unsubstituted or substituted C₁₋₁₀alkyl, unsubstituted or substituted C₃₋₁₀cycloalkyl, unsubstituted or substituted C₆₋₁₀aryl, unsubstituted or substituted C₅₋₁₀heteroaryl, unsubstituted or substituted CR^{f}₂—C₆₋₁₀aryl, and R¹⁰ and R¹¹ cyclized to form a unsubstituted or substituted ring having 3-8 ring members; wherein each R¹⁰, R¹¹, and the ring having 3-8 ring members is optionally substituted with one to five R^{e} groups;
R^{a} and R^{b} are each independently selected from the group consisting of C₁₋₁₀alkyl, C₃₋₈cycloalkyl, and C₆₋₁₀aryl;
R^{c} and R^{d} are each independently selected from the group consisting of unsubstituted or substituted C₁₋₁₀alkyl, unsubstituted or substituted C₃₋₈cycloalkyl, and C₆₋₁₀aryl; wherein each R^{C} and R^{d} is optionally substituted with one to five R^{e} groups;
R^{e} is selected from the group consisting of halogen, OH, C₁₋₁₀alkyl, O—C₁₋₁₀alkyl, C₁₋₁₀haloalkyl, O—C₁₋₁₀haloalkyl, cyano, C₃₋₈cycloalkyl, C₆₋₁₀aryl, and NR^{g}R^{h};
R^{f} is selected from the group consisting of hydrogen, unsubstituted or substituted C₁₋₁₀alkyl, unsubstituted or substituted C₁₋₁₀haloalkyl, unsubstituted or substituted C₃₋₈cycloalkyl; wherein each R^{f} is optionally substituted with one to five R^{e} groups; and
R^{g} and R^{h} are each independently selected from the group consisting of C₁₋₁₀alkyl, C₃₋₈cycloalkyl, and C₆₋₁₀aryl,
or a pharmaceutically acceptable salt thereof.

37. The compound of claim 36, wherein:
R¹ is selected from the group consisting of hydrogen, halogen, and C₁₋₁₀alkyl;
R³ is OR⁹ or NR¹⁰R¹¹;
R⁴, R⁵, R⁶, and R⁷ are each independently selected from the group consisting of hydrogen and C₁₋₁₀alkyl;
R⁸ is selected from the group consisting of unsubstituted or substituted C₁₋₁₀alkyl, unsubstituted or substituted C₁₋₁₀haloalkyl, unsubstituted or substituted O—C moalkyl, unsubstituted or substituted C₆₋₁₀aryl, unsubstituted or substituted O—C₆₋₁₀aryl, unsubstituted or substituted C₃₋₈cycloalkyl, unsubstituted or substituted O—C₃₋₈cycloalkyl, unsubstituted or substituted C₂₋₇heterocycloalkyl, and unsubstituted or substituted NR^{c}R^{d}; wherein each R⁸ is optionally substituted with one to five R^{e} groups;
R⁹ is unsubstituted or substituted C₁₋₁₀alkyl; wherein each C₁₋₁₀alkyl is optionally substituted with one to five R^{e} groups;
R¹⁰ and R¹¹ are each independently selected from the group consisting of hydrogen, unsubstituted or substituted C₁₋₁₀alkyl, and R¹⁰ and R¹¹ cyclized to form a unsubstituted or substituted ring having 3-8 ring members; wherein each R¹⁰, R¹¹, and the ring having 3-8 ring members is optionally substituted with one to five R^{e} groups;
R^{c} and R^{d} are each independently unsubstituted or substituted C₁₋₁₀alkyl; wherein each R^{c} and R^{d} is optionally substituted with one to five R^{e} groups; and
R^{e} is selected from the group consisting of halogen, OH, C₁₋₁₀alkyl, cyano and C₃₋₈cycloalkyl;
or a pharmaceutically acceptable salt thereof.

38. The compound of claim 36, wherein
R¹ is hydrogen or C₁₋₁₀alkyl;
R⁴, R⁵ and R⁶ are each hydrogen;
R⁷ is hydrogen or C₁₋₁₀alkyl;
R⁸ is selected from the group consisting of C₁₋₁₀alkyl, O—C₆₋₁₀aryl, C₃₋₈cycloalkyl, O—C₃₋₈cycloalkyl, C₂₋₇heterocycloalkyl, and NR^{c}R^{d}, wherein each R⁸ is optionally substituted with one to five R^{e} groups;
R⁹ is unsubstituted C₁₋₁₀alkyl;
R¹⁰ and R¹¹ are each independently selected from the group consisting of hydrogen, C₁₋₁₀alkyl, and C₃₋₁₀cycloalkyl, or R¹⁰ and R¹¹ are cyclized to form a ring having 3-8 ring members, wherein each R¹⁰, R¹¹, and the ring having 3-8 ring members is optionally substituted with one to five R^{e} groups;
R^{c} and R^{d} are each independently C₁₋₁₀alkyl, wherein each R^{c} and R^{d} is optionally substituted with one to five R^{e} groups;
R^{e} is selected from the group consisting of halogen, OH, C₁₋₁₀alkyl, C₁₋₁₀haloalkyl, cyano, C₃₋₈cycloalkyl, and C₆₋₁₀aryl;
or a pharmaceutically acceptable salt thereof.

39. The compound of claim 36, wherein R¹, R⁴, R⁵, R⁶, and R⁷ are each hydrogen.

40. The compound of claim 36, wherein R³ is OR⁹.

41. The compound of claim 40, wherein R⁹ is unsubstituted C₁₋₁₀alkyl.

42. The compound of claim 40, wherein R⁹ is ethyl.

43. The compound of claim 40, wherein the compound is selected from the group consisting of: 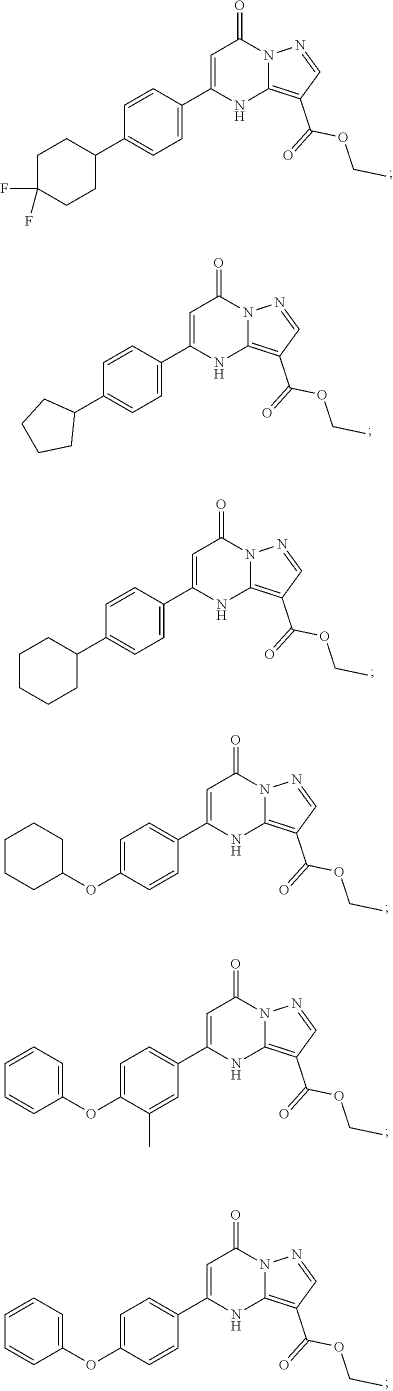 [SMILES: CCOC(=O)c1cnn2c(=O)cc(-c3ccc(C4CCC(F)(F)CC4)cc3)[nH]c12 ; CCOC(=O)c1cnn2c(=O)cc(-c3ccc(C4CCCC4)cc3)[nH]c12 ; CCOC(=O)c1cnn2c(=O)cc(-c3ccc(C4CCCCC4)cc3)[nH]c12 ; CCOC(=O)c1cnn2c(=O)cc(-c3ccc(OC4CCCCC4)cc3)[nH]c12 ; CCOC(=O)c1cnn2c(=O)cc(-c3ccc(Oc4ccccc4)c(C)c3)[nH]c12 ; CCOC(=O)c1cnn2c(=O)cc(-c3ccc(Oc4ccccc4)cc3)[nH]c12] 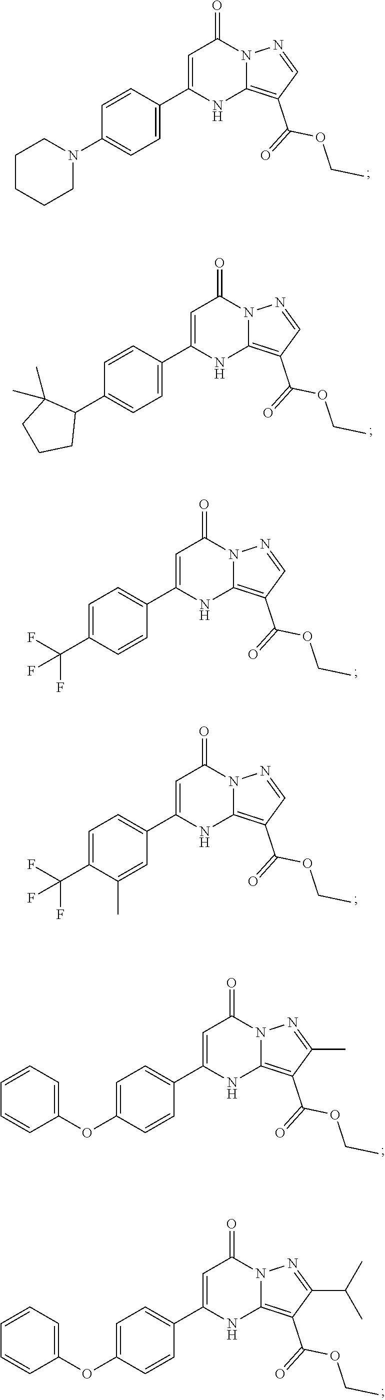 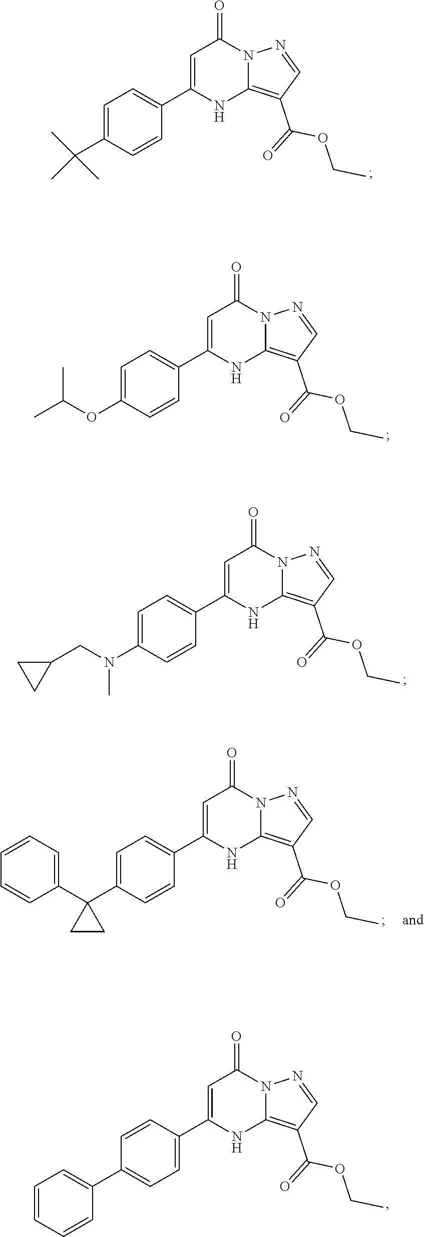
or a pharmaceutically acceptable salt thereof.

44. The compound of claim 36, wherein R³ is NR¹⁰R¹¹.

45. The compound of claim 44, wherein R¹⁰ and R¹¹ are each independently selected from the group consisting of hydrogen, unsubstituted C₃₋₁₀cycloalkyl and C₁₋₁₀alkyl optionally substituted with R^{e}, wherein R^{e} is OH.

46. The compound of claim 45, wherein R¹⁰ and R¹¹ are each independently selected from the group consisting of hydrogen, unsubstituted C₃cycloalkyl and C₁₋₂alkyl optionally substituted with R^{e}, wherein R^{e} is OH.

47. The compound of claim 44, wherein R¹⁰ and R¹¹ are cyclized to form a ring having 3-8 ring members optionally substituted with one to two R^{e} groups selected from the group consisting of halogen, OH, C₁₋₁₀alkyl, C₁₋₁₀haloalkyl and cyano.

48. The compound of claim 47, wherein R¹⁰ and R¹¹ are cyclized to form a ring having 4-5 ring members optionally substituted with one to two R^{e} groups selected from the group consisting of halogen, OH, C₁₋₁₀alkyl, C₁₋₁₀haloalkyl and cyano.

49. The compound of claim 47, wherein R¹⁰ and R¹¹ are cyclized to form a ring having 4 ring members optionally substituted with one to two R^{e} groups selected from the group consisting of halogen, OH, C₁alkyl, C₁haloalkyl and cyano.

50. The compound of claim 47, wherein R¹⁰ and R¹¹ are cyclized to form a ring having 5 ring members optionally substituted with one to two R^{e} groups selected from the group consisting of halogen and cyano.

51. The compound of claim 44, wherein the compound is selected from the group consisting of: 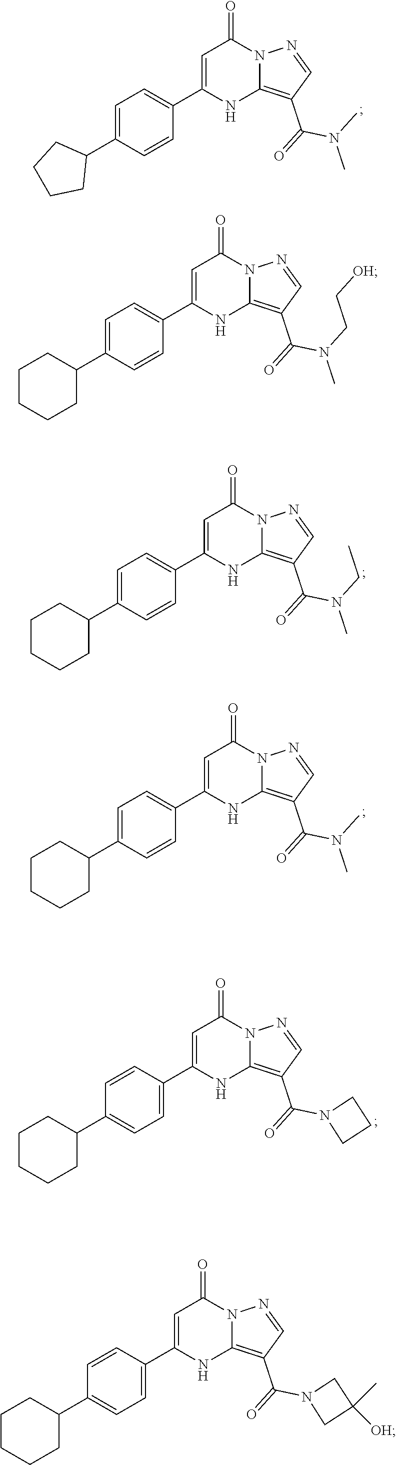 [SMILES: CN(C)C(=O)c1cnn2c(=O)cc(-c3ccc(C4CCCC4)cc3)[nH]c12 ; CN(CCO)C(=O)c1cnn2c(=O)cc(-c3ccc(C4CCCCC4)cc3)[nH]c12 ; CCN(C)C(=O)c1cnn2c(=O)cc(-c3ccc(C4CCCCC4)cc3)[nH]c12 ; CN(C)C(=O)c1cnn2c(=O)cc(-c3ccc(C4CCCCC4)cc3)[nH]c12 ; O=C(c1cnn2c(=O)cc(-c3ccc(C4CCCCC4)cc3)[nH]c12)N1CCC1 ; CC1(O)CN(C(=O)c2cnn3c(=O)cc(-c4ccc(C5CCCCC5)cc4)[nH]c23)C1] 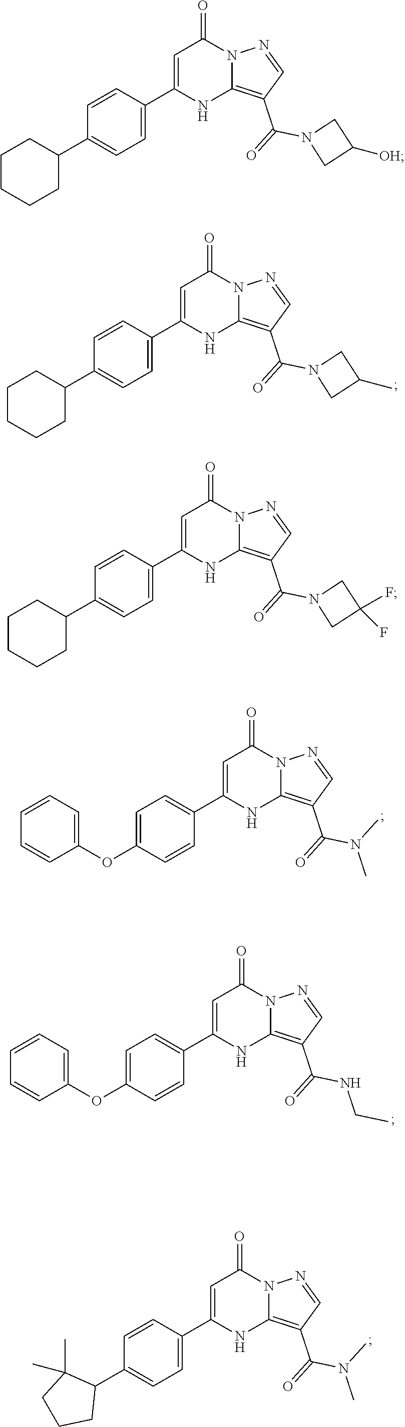 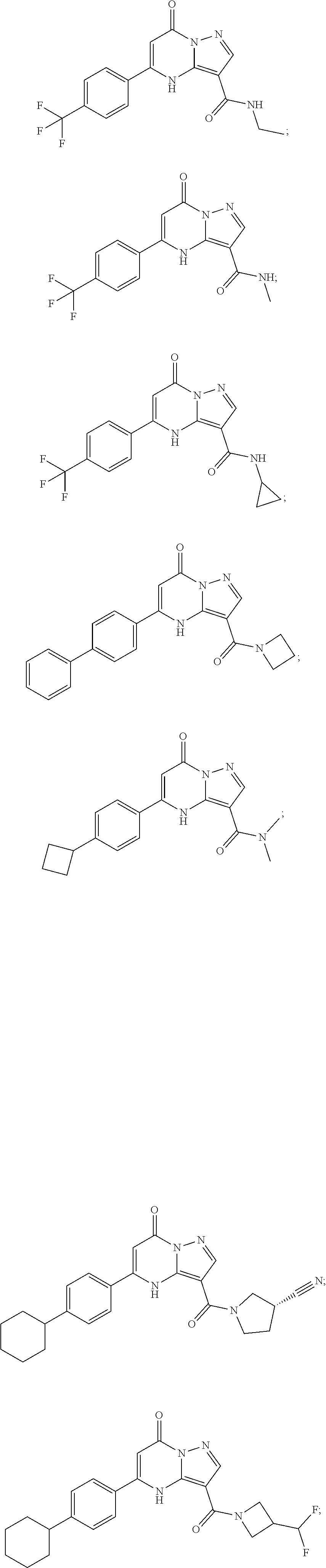 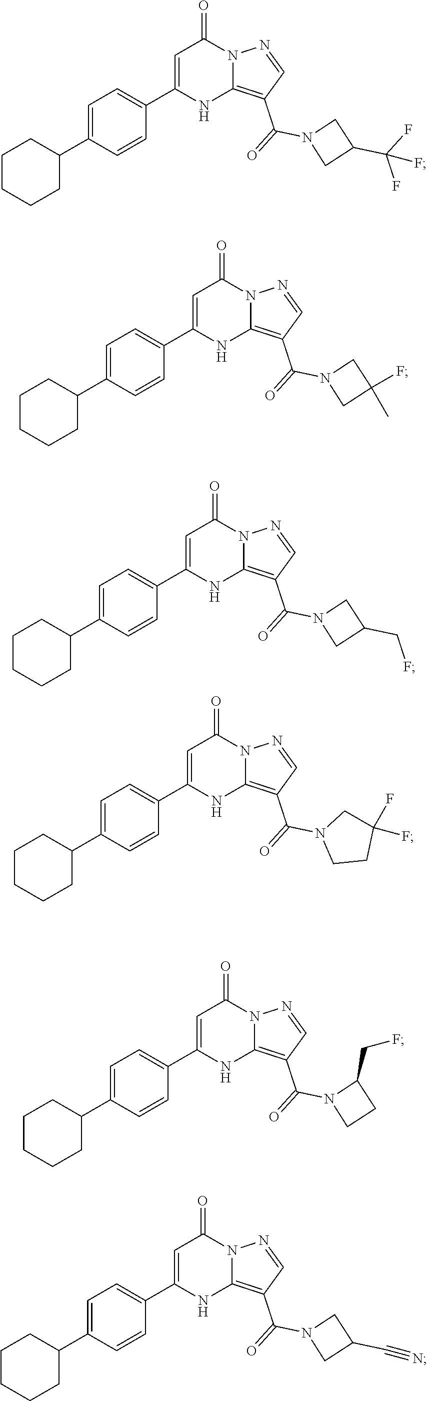 [SMILES: O=C(c1cnn2c(=O)cc(-c3ccc(C4CCCCC4)cc3)[nH]c12)N1CC(C(F)(F)F)C1 ; CC1(F)CN(C(=O)c2cnn3c(=O)cc(-c4ccc(C5CCCCC5)cc4)[nH]c23)C1 ; O=C(c1cnn2c(=O)cc(-c3ccc(C4CCCCC4)cc3)[nH]c12)N1CC(CF)C1 ; O=C(c1cnn2c(=O)cc(-c3ccc(C4CCCCC4)cc3)[nH]c12)N1CCC(F)(F)C1 ; O=C(c1cnn2c(=O)cc(-c3ccc(C4CCCCC4)cc3)[nH]c12)N1CC[C@@H]1CF ; N#CC1CN(C(=O)c2cnn3c(=O)cc(-c4ccc(C5CCCCC5)cc4)[nH]c23)C1] 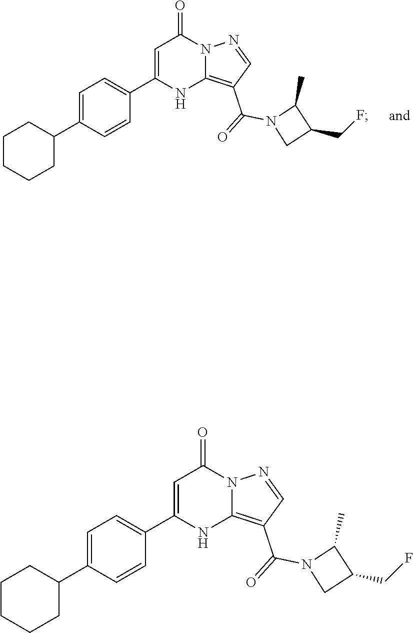 [SMILES: C[C@H]1[C@@H](CF)CN1C(=O)c1cnn2c(=O)cc(-c3ccc(C4CCCCC4)cc3)[nH]c12 ; "and" ; C[C@@H]1[C@H](CF)CN1C(=O)c1cnn2c(=O)cc(-c3ccc(C4CCCCC4)cc3)[nH]c12]
or a pharmaceutically acceptable salt thereof.

52. The compound of claim 36, wherein R⁸ is selected from the group consisting of C₁₋₁₀alkyl, O—C₆₋₁₀aryl, C₃₋₈cycloalkyl, O—C₃₋₈cycloalkyl, C₂₋₇heterocycloalkyl, and NR^{c}R^{d}, wherein R^{c} and R^{d} are each independently C₁₋₁₀alkyl, wherein R⁸, R^{c} and R^{d} are each independently optionally substituted with one to two R^{e} groups selected from the group consisting of halogen, OH, C₁₋₁₀alkyl, C₁₋₁₀haloalkyl, cyano, C₃₋₈cycloalkyl, and C₆₋₁₀aryl.

53. The compound of claim 36, wherein R⁸ is C₄₋₆cycloalkyl optionally substituted with one or two substituents selected from the group consisting of halogen and C₁₋₂alkyl.

54. The compound of claim 36, wherein R⁸ is C₅cycloalkyl optionally substituted with one or two C₁₋₂alkyl.

55. The compound of claim 36, wherein R⁸ is unsubstituted C₆cycloalkyl.

56. The compound of claim 36, wherein R⁸ is selected from the group consisting of:

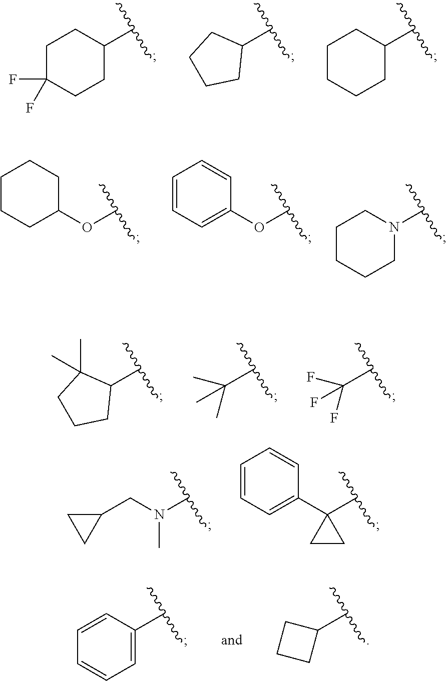

57. The compound of claim 36, wherein
R¹, R⁴, R⁵, R⁶, and R⁷ are each hydrogen;
R³ is OR⁹, wherein R⁹ is ethyl; and
R⁸ is C₄₋₆cycloalkyl optionally substituted with one or two substituents selected from the group consisting of halogen and C₁₋₂alkyl.

58. The compound of claim 36, wherein
R¹, R⁴, R⁵, R⁶, and R⁷ are each hydrogen;
R³ is R³ is NR¹⁰R¹¹, wherein R¹⁰ and R¹¹ are cyclized to form a ring having 4-5 ring members optionally substituted with one to two R^{e} groups selected from the group consisting of halogen, OH, C₁₋₁₀alkyl, C₁₋₁₀haloalkyl and cyano; and
R⁸ is C₄₋₆cycloalkyl optionally substituted with one or two substituents selected from the group consisting of halogen and C₁₋₂alkyl.

59. The compound of claim 36, wherein said compound is selected from the group consisting of
Ethyl 5-(4-(4,4-difluorocyclohexyl)phenyl)-7-oxo-4,7-dihydropyrazolo[1,5-a]pyrimidine-3-carboxylate;
Ethyl 5-(4-cyclopentylphenyl)-7-oxo-4,7-dihydropyrazolo[1,5-a]pyrimidine-3-carboxylate;
5-(4-Cyclopentylphenyl)-N,N-dimethyl-7-oxo-4,7-dihydropyrazolo[1,5-a]pyrimidine-3-carb oxamide;
Ethyl 5-(4-cyclohexylphenyl)-7-oxo-4,7-dihydropyrazolo[1,5-a] pyrimidine-3-carboxylate;
5-(4-cyclohexylphenyl)-N-(2-hydroxyethyl)-N-methyl-7-oxo-4,7-dihydropyrazolo[1,5-a] pyrimidine-3-carboxamide;
5-(4-Cyclohexylphenyl)-N-ethyl-N-methyl-7-oxo-4,7-dihydropyrazolo[1,5-a] pyrimidine-3-carboxamide;
5-(4-Cyclohexylphenyl)-N,N-dimethyl-7-oxo-4,7-dihydropyrazolo[1,5-a] pyrimidine-3-carb oxamide;
3-(Azetidine-1-carbonyl)-5-(4-cyclohexylphenyl)pyrazolo[1,5-a] pyrimidin-7(4H)-one;
5-(4-Cyclohexylphenyl)-3-(3-hydroxy-3-methylazetidine-1-carbonyl)pyrazolo[1,5-a]pyrimidin-7(4H)-one;
5-(4-Cyclohexylphenyl)-3-(3-hydroxyazetidine-1-carbonyl)pyrazolo[1,5-a]pyrimidin-7(4H)-one;
5-(4-Cyclohexylphenyl)-3-(3-methylazetidine-1-carbonyl)pyrazolo[1,5-a]pyrimidin-7(4H)-one;
5-(4-Cyclohexylphenyl)-3-(3,3-difluoroazetidine-1-carbonyl)pyrazolo[1,5-a]pyrimidin-7(4H)-one;
Ethyl 5-(4-(Cyclohexyloxy)phenyl)-7-oxo-4,7-dihydropyrazolo[1,5-a]pyrimidine-3-carboxylate;
Ethyl 5-(3-methyl-4-phenoxyphenyl)-7-oxo-4,7-dihydropyrazolo[1,5-a]pyrimidine-3-carboxylate;
Ethyl 7-oxo-5-(4-phenoxyphenyl)-4,7-dihydropyrazolo[1,5-a]pyrimidine-3-carb oxylate;
N,N-Dimethyl-7-oxo-5-(4-phenoxyphenyl)-4,7-dihydropyrazolo[1,5-a]pyrimidine-3-carboxamide;
N-Ethyl-7-oxo-5-(4-phenoxyphenyl)-4,7-dihydropyrazolo[1,5-a]pyrimidine-3-carboxamide;
Ethyl 7-oxo-5-(4-(piperidin-1-yl)phenyl)-4,7-dihydropyrazolo[1,5-a]pyrimidine-3-carboxylate;
(S)-Ethyl 5-(4-(2,2-dimethylcyclopentyl)phenyl)-7-oxo-4,7-dihydropyrazolo[1,5-a]pyrimidine-3-carboxylate;
(R)-ethyl 5-(4-(2,2-dimethylcyclopentyl)phenyl)-7-oxo-4,7-dihydropyrazolo[1,5-a]pyrimidine-3-carboxylate;
(R)-5-(4-(2,2-Dimethylcyclopentyl)phenyl)-N,N-dimethyl-7-oxo-4,7-dihydropyrazolo[1,5-a]pyrimidine-3-carboxamide;
(S)-5-(4-(2,2-dimethylcyclopentyl)phenyl)-N,N-dimethyl-7-oxo-4,7-dihydropyrazolo[1,5-a]pyrimidine-3-carboxamide;
Ethyl 7-oxo-5-(4-(trifluoromethyl)phenyl)-4,7-dihydropyrazolo[1,5-a]pyrimidine-3-carboxylate;
N-Ethyl-7-oxo-5-(4-(trifluoromethyl)phenyl)-4,7-dihydropyrazolo[1,5-a]pyrimidine-3-carboxamide;
N-methyl-7-oxo-5-(4-(trifluoromethyl)phenyl)-4,7-dihydropyrazolo[1,5-a]pyrimidine-3-carboxamide;
Ethyl 5-(3-methyl-4-(trifluoromethyl)phenyl)-7-oxo-4,7-dihydropyrazolo[1,5-a]pyrimidine-3-carboxylate;
N-Cyclopropyl-7-oxo-5-(4-(trifluoromethyl)phenyl)-4,7-dihydropyrazolo[1,5-a]pyrimidine-3-carboxamide;
Ethyl 2-methyl-7-oxo-5-(4-phenoxyphenyl)-4,7-dihydropyrazolo[1,5-a]pyrimidine-3-carboxylate;
Ethyl 2-isopropyl-7-oxo-5-(4-phenoxyphenyl)-4,7-dihydropyrazolo[1,5-a]pyrimidine-3-carboxylate;
Ethyl 5-(4-(tert-butyl)phenyl)-7-oxo-4,7-dihydropyrazolo[1,5-a]pyrimidine-3-carboxylate;
Ethyl 5-(4-((cyclopropylmethyl)(methyl)amino)phenyl)-7-oxo-4,7-dihydro pyrazolo[1,5-a]pyrimidine-3-carboxylate;
Ethyl 7-oxo-5-(4-(1-phenylcyclopropyl)phenyl)-4,7-dihydropyrazolo[1,5-a]pyrimidine-3-carboxylate;
Ethyl 5-([1,1′-biphenyl]-4-yl)-7-oxo-4,7-dihydropyrazolo[1,5-a]pyrimidine-3-carboxylate;
5-([1,1′-Biphenyl]-4-yl)-3-(azetidine-1-carbonyl)pyrazolo[1,5-a]pyrimidin-7(4H)-one;
5-(4-Cyclobutylphenyl)-N,N-dimethyl-7-oxo-4,7-dihydropyrazolo[1,5-a]pyrimidine-3-carboxamide;
(R)-1-(5-(4-Cyclohexylphenyl)-7-oxo-4,7-dihydropyrazolo[1,5-a]pyrimidine-3-carbonyl)pyrrolidine-3-carbonitrile;
5-(4-Cyclohexylphenyl)-3-(3-(difluoromethyl)azetidine-1-carbonyl)pyrazolo[1,5-a]pyrimidin-7(4H)-one;
5-(4-Cyclohexylphenyl)-3-(3-(trifluoromethyl)azetidine-1-carbonyl)pyrazolo[1,5-a]pyrimidin-7(4H)-one;
5-(4-Cyclohexylphenyl)-3-(3-fluoro-3-methylazetidine-1-carbonyl)pyrazolo[1,5-a]pyrimidin-7(4H)-one;
5-(4-Cyclohexylphenyl)-3-(3-(fluoromethyl)azetidine-1-carbonyl)pyrazolo[1,5-a]pyrimidin-7(4H)-one;
5-(4-Cyclohexylphenyl)-3-(3,3-difluoropyrrolidine-1-carbonyl)pyrazolo[1,5-a]pyrimidin-7(4H)-one;
(R)-5-(4-cyclohexylphenyl)-3-(2-(fluoromethyl)azetidine-1-carbonyl)pyrazolo[1,5-a]pyrimidin-7(4H)-one;
1-(5-(4-Cyclohexylphenyl)-7-oxo-4,7-dihydropyrazolo[1,5-a]pyrimidine-3-carbonyl)azetidine-3-carbonitrile;
5-(4-Cyclohexylphenyl)-3-((2R,3R)-3-(fluoromethyl)-2-methylazetidine-1-carbonyl) pyrazolo[1,5-a]pyrimidin-7(4H)-on;
5-(4-cyclohexylphenyl)-3-((2S,3S)-3-(fluoro methyl)-2-methylazetidine-1-carbonyl)pyrazolo[1,5-a]pyrimidin-7(4H)-one, and pharmaceutically acceptable salts thereof.

60. A pharmaceutical composition comprising a compound of claim 36, and a therapeutically inert carrier.

61. A method for the therapeutic treatment of cancer in a subject, which method comprises administering to said subject an effective amount of a compound of formula (I): 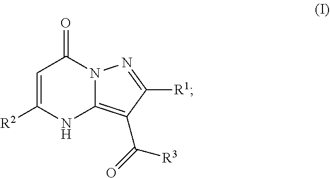
wherein:
R¹ is selected from the group consisting of hydrogen, halogen, C₁₋₁₀alkyl, and C₁₋₁₀haloalkyl;
R² is 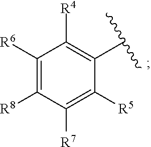
R³ is OR⁹ or NR¹⁰NR¹¹, wherein when R² is C₅₋₁₀heteroaryl and R³ is NR¹⁰R¹¹, then each of R¹⁰ and R¹¹ is not hydrogen;
R⁴, R⁵, R⁶, and R⁷ are each independently selected from the group consisting of hydrogen, halogen, C₁₋₁₀alkyl, C₁₋₁₀haloalkyl, O—C₁₋₁₀alkyl, and NR^{a}R^{b};
R⁸ is selected from the group consisting of unsubstituted or substituted C₁₋₁₀alkyl, unsubstituted or substituted C₁₋₁₀haloalkyl, unsubstituted or substituted O—C₁₋₁₀alkyl, unsubstituted or substituted O—C₁₋₁₀haloalkyl, unsubstituted or substituted C₆₋₁₀aryl, unsubstituted or substituted O—C₆-10^{aryl}, unsubstituted or substituted C₃₋₈cycloalkyl, unsubstituted or substituted O—C₃₋₈cycloalkyl, unsubstituted or substituted C₂₋₇heterocycloalkyl, unsubstituted or substituted C₅₋₁₀heteroaryl; and unsubstituted or substituted NR^{c}R^{d}; wherein each R⁸ is optionally substituted with one to five R^{e} groups;
R⁹ is selected from the group consisting of unsubstituted or substituted C₁₋₁₀alkyl, unsubstituted or substituted C₃₋₈cycloalkyl, unsubstituted or substituted C₆₋₁₀aryl, and unsubstituted or substituted C₅₋₁₀heteroaryl; wherein each R⁹ is optionally substituted with one to five R^{e} groups;
R¹⁰ and R¹¹ are each independently selected from the group consisting of hydrogen, unsubstituted or substituted C₁₋₁₀alkyl, unsubstituted or substituted C₃₋₁₀cycloalkyl, unsubstituted or substituted C₆₋₁₀aryl, unsubstituted or substituted C₅₋₁₀heteroaryl, unsubstituted or substituted CR^{f}₂—C₆₋₁₀aryl, and R¹⁰ and R¹¹ cyclized to form a unsubstituted or substituted ring having 3-8 ring members; wherein each R¹⁰, R¹¹, and the ring having 3-8 ring members is optionally substituted with one to five R^{e} groups;
R^{a} and R^{b} are each independently selected from the group consisting of C₁₋₁₀alkyl, C₃₋₈cycloalkyl, and C₆₋₁₀aryl;
R^{c} and R^{d} are each independently selected from the group consisting of unsubstituted or substituted C₁₋₁₀alkyl, unsubstituted or substituted C₃₋₈cycloalkyl, and C₆₋₁₀aryl; wherein each R^{c} and R^{d} is optionally substituted with one to five R^{e} groups;
R^{e} is selected from the group consisting of halogen, OH, C₁₋₁₀alkyl, O—C₁₋₁₀alkyl, C₁₋₁₀haloalkyl, O—C₁₋₁₀haloalkyl, cyano, C₃₋₈cycloalkyl, C₆₋₁₀aryl, and NR^{g}R^{h};
R^{f} is selected from the group consisting of hydrogen, unsubstituted or substituted C₁₋₁₀alkyl, unsubstituted or substituted C₁₋₁₀haloalkyl, unsubstituted or substituted C₃₋₈cycloalkyl; wherein each R^{f} is optionally substituted with one to five R^{e} groups; and
R^{g} and R^{h} are each independently selected from the group consisting of C₁₋₁₀alkyl, C₃₋₈cycloalkyl, and C₆₋₁₀aryl,
or a pharmaceutically acceptable salt thereof.

62. The method of claim 61, wherein the compound is selected from the group consisting of Ethyl 5-(4-(4,4-difluorocyclohexyl)phenyl)-7-oxo-4,7-dihydropyrazolo[1,5-a]pyrimidine-3-carboxylate;
Ethyl 5-(4-cyclopentylphenyl)-7-oxo-4,7-dihydropyrazolo[1,5-a]pyrimidine-3-carboxylate;
5-(4-Cyclopentylphenyl)-N,N-dimethyl-7-oxo-4,7-dihydropyrazolo[1,5-a]pyrimidine-3-carboxamide;
Ethyl 5-(4-cyclohexylphenyl)-7-oxo-4,7-dihydropyrazolo[1,5-a]pyrimidine-3-carboxylate;
5-(4-cyclohexylphenyl)-N-(2-hydroxyethyl)-N-methyl-7-oxo-4,7-dihydropyrazolo[1,5-a]pyrimidine-3-carboxamide;
5-(4-Cyclohexylphenyl)-N-ethyl-N-methyl-7-oxo-4,7-dihydropyrazolo[1,5-c]pyrimidine-3-carboxamide;
5-(4-Cyclohexylphenyl)-N,N-dimethyl-7-oxo-4,7-dihydropyrazolo[1,5-a]pyrimidine-3-carb oxamide;
3-(Azetidine-1-carbonyl)-5-(4-cyclohexylphenyl)pyrazolo[1,5-a]pyrimidin-7(4H)-one;
5-(4-Cyclohexylphenyl)-3-(3-hydroxy-3-methylazetidine-1-carbonyl)pyrazolo[1,5-a]pyrimidin-7(4H)-one;
5-(4-Cyclohexylphenyl)-3-(3-hydroxyazetidine-1-carbonyl)pyrazolo[1,5-a]pyrimidin-7(4H)-one;
5-(4-Cyclohexylphenyl)-3-(3-methylazetidine-1-carbonyl)pyrazolo[1,5-a]pyrimidin-7(4H)-one;
5-(4-Cyclohexylphenyl)-3-(3,3-difluoroazetidine-1-carbonyl)pyrazolo[1,5-a]pyrimidin-7(4H)-one;
Ethyl 5-(4-(Cyclohexyloxy)phenyl)-7-oxo-4,7-dihydropyrazolo[1,5-a]pyrimidine-3carboxylate;
Ethyl 5-(3-methyl-4-phenoxyphenyl)-7-oxo-4,7-dihydropyrazolo[1,5-a]pyrimidine-3-carboxylate;
Ethyl 7-oxo-5-(4-phenoxyphenyl)-4,7-dihydropyrazolo[1,5-a]pyrimidine-3-carboxylate;
N,N-Dimethyl-7-oxo-5-(4-phenoxyphenyl)-4,7-dihydropyrazolo[1,5-a]pyrimidine-3-carboxamide;
N-Ethyl-7-oxo-5-(4-phenoxyphenyl)-4,7-dihydropyrazolo[1,5-a]pyrimidine-3-carboxamide;
Ethyl 7-oxo-5-(4-(piperidin-1-yl)phenyl)-4,7-dihydropyrazolo[1,5-a]pyrimidine-3-carboxylate;
(5)-Ethyl 5-(4-(2,2-dimethylcyclopentyl)phenyl)-7-oxo-4,7-dihydropyrazolo[1,5-a]pyrimidine-3-carboxylate;
(R)-ethyl 5-(4-(2,2-dimethylcyclopentyl)phenyl)-7-oxo-4,7-dihydropyrazolo[1,5-a]pyrimidine-3-carboxylate;
(R)-5-(4-(2,2-Dimethylcyclopentyl)phenyl)-N,N-dimethyl-7-oxo-4,7-dihydropyrazolo[1,5-a]pyrimidine-3-carboxamide;
(S)-5-(4-(2,2-dimethylcyclopentyl)phenyl)-N,N-dimethyl-7-oxo-4,7-dihydropyrazolo[1,5-a]pyrimidine-3-carboxamide;
Ethyl 7-oxo-5-(4-(trifluoromethyl)phenyl)-4,7-dihydropyrazolo[1,5-a]pyrimidine-3-carboxylate;
N-Ethyl-7-oxo-5-(4-(trifluoromethyl)phenyl)-4,7-dihydropyrazolo[1,5-a]pyrimidine-3-carboxamide;
N-methyl-7-oxo-5-(4-(trifluoromethyl)phenyl)-4,7-dihydropyrazolo[1,5-a]pyrimidine-3-carboxamide;
Ethyl 5-(3-methyl-4-(trifluoromethyl)phenyl)-7-oxo-4,7-dihydropyrazolo[1,5-a]pyrimidine-3-carboxylate;
N-Cyclopropyl-7-oxo-5-(4-(trifluoromethyl)phenyl)-4,7-dihydropyrazolo[1,5-a]pyrimidine-3-carboxamide;
Ethyl 2-methyl-7-oxo-5-(4-phenoxyphenyl)-4,7-dihydropyrazolo[1,5-a]pyrimidine-3-carboxylate;
Ethyl 2-isopropyl-7-oxo-5-(4-phenoxyphenyl)-4,7-dihydropyrazolo[1,5-a]pyrimidine-3-carboxylate;
Ethyl 5-(4-(tert-butyl)phenyl)-7-oxo-4,7-dihydropyrazolo[1,5-a]pyrimidine-3-carboxylate;
Ethyl 5-(4-isopropoxyphenyl)-7-oxo-4,7-dihydropyrazolo[1,5-a]pyrimidine-3-carboxylate;
Ethyl 5-(4-((cyclopropylmethyl)(methyl)amino)phenyl)-7-oxo-4,7-dihydro pyrazolo[1,5-a]pyrimidine-3-carboxylate;
Ethyl 7-oxo-5-(4-(1-phenylcyclopropyl)phenyl)-4,7-dihydropyrazolo[1,5-a]pyrimidine-3-carboxylate;
Ethyl 5-([1,1′-biphenyl]-4-yl)-7-oxo-4,7-dihydropyrazolo[1,5-a]pyrimidine-3-carboxylate;
5-([1,1′-Biphenyl]-4-yl)-3-(azetidine-1-carbonyl)pyrazolo[1,5-a]pyrimidin-7(4H)-one;
5-(4-Cyclobutylphenyl)-N,N-dimethyl-7-oxo-4,7-dihydropyrazolo[1,5-a]pyrimidine-3-carboxamide;
(R)-1-(5-(4-Cyclohexylphenyl)-7-oxo-4,7-dihydropyrazolo[1,5-a]pyrimidine-3-carbonyl)pyrrolidine-3-carbonitrile;
5-(4-Cyclohexylphenyl)-3-(3-(difluoromethyl)azetidine-1-carbonyl)pyrazolo[1,5-a]pyrimidin-7(4H)-one;
5-(4-Cyclohexylphenyl)-3-(3-(trifluoromethyl)azetidine-1-carbonyl)pyrazolo[1,5-a]pyrimidin-7(4H)-one;
5-(4-Cyclohexylphenyl)-3-(3-fluoro-3-methylazetidine-1-carbonyl)pyrazolo[1,5-a]pyrimidin-7(4H)-one;
5-(4-Cyclohexylphenyl)-3-(3-(fluoromethyl)azetidine-1-carbonyl)pyrazolo[1,5-a]pyrimidin-7(4H)-one;
5-(4-Cyclohexylphenyl)-3-(3,3-difluoropyrrolidine-1-carbonyl)pyrazolo[1,5-a]pyrimidin-7(4H)-one;
(R)-5-(4-cyclohexylphenyl)-3-(2-(fluoromethyl)azetidine-1-carbonyl)pyrazolo[1,5-a]pyrimidin-7(4H)-one;
1-(5-(4-Cyclohexylphenyl)-7-oxo-4,7-dihydropyrazolo[1,5-a]pyrimidine-3-carbonyl)azetidine-3-carbonitrile;
5-(4-Cyclohexylphenyl)-3-((2R,3R)-3-(fluoromethyl)-2-methylazetidine-1-carbonyl) pyrazolo[1,5-a]pyrimidin-7(4H)-on; and
5-(4-cyclohexylphenyl)-3-((2S,3S)-3-(fluoro methyl)-2-methylazetidine-1-carbonyl)pyrazolo[1,5-a]pyrimidin-7(4H)-one,
or pharmaceutically acceptable salts thereof.

63. The method of claim 61, wherein the cancer is a solid tumor.

64. The method of claim 61, wherein the cancer is selected from the group consisting of lung, liver, ovarian, breast and squamous cancer.

65. A process of making the compound of claim 36.
